(19) United States
(12) Patent Application Publication
(10) Pub. No.: US 2025/0145570 A1
(43) Pub. Date: May 8, 2025

(54) DHODH INHIBITOR POLYMORPH

(71) Applicant: Aslan Pharmaceuticals Pte Ltd, Singapore (SG)

(72) Inventor: Robert MOORE, Singapore (SG)

(21) Appl. No.: 18/835,823

(22) PCT Filed: Feb. 2, 2023
(86) PCT No.: PCT/SG2023/050059

§ 371 Date: Aug. 5, 2024

(30) Foreign Application Priority Data
Feb. 3, 2022 (SG) 10202201038X
Jun. 13, 2022 (GB) 2208572.4

(51) Int. Cl. C07D 213/80; A61K 31/455

(52) U.S. Cl. CPC C07D 213/80; A61K 31/455

(57) ABSTRACT

The present disclosure relates to physical forms of a DHODH inhibitor, such as a stable crystalline form of and compositions comprising the same. Also provided is the use of the physical form (such as a polymorph) or composition thereof in treatment, such as the treatment of a viral disease or an autoimmune disease or disorder.

The present disclosure relates to physical forms of a DHODH inhibitor, such as a stable crystalline form of and compositions comprising the same. Also provided is the use of the physical form (such as a polymorph) or composition in treatment such as the treatment of a viral disease or an autoimmune disease or disorder.

### BACKGROUND

2-(3, 5-difluoro-3′-methoxybiphenyl-4-ylamino) nicotinic acid (farudodstat, previously known as ASLAN003) is a next generation DHODH inhibitor.

Dihydroorotate dehydrogenase (DHODH) is the enzyme that catalyses the fourth step in the pyrimidine biosynthetic pathway namely the conversion of dihydroorotate to orotate concomitantly with an electron transfer to ubiquinone (cofactor Q) via a flavin mononucleotide intermediate (Loffler MoI Cell Biochem, 1997). In contrast to parasites (Plasmodium falciparum) (McRobert et al MoI Biochem Parasitol 2002) and bacteria (E. coli) which exclusively have this de novo pathway as the source of pyrimidines, mammal cells have an additional salvage pathway.

During homeostatic proliferation the salvage pathway, which is independent of DHODH, seems sufficient for the cellular supply with pyrimidine bases. However, in cells with a high turnover the de novo pathway is required to proliferate. In these cells, DHODH inhibition stops the cell cycle progression by suppressing DNA and RNA synthesis and ultimately cell proliferation (Breedveld F. C. Ann Rheum Dis 2000).

There are some suggestions that inhibition of mitochondrial cytochrome bc1, a component of the electron transport chain complex III, leads to activation of p53, followed by apoptosis induction. The mitochondrial respiratory chain is coupled to the de novo pyrimidine biosynthesis pathway via the mitochondrial enzyme dihydroorotate dehydrogenase (DHODH).

Thus, administering farudodstat to cells with a high metabolic burden may inhibit normal functioning of the cell, and promote apoptosis/removal of these cells by the body's natural mechanisms. The latter is useful in virus infected cells and cancer cells. In addition, farudodstat has activity against the underlying cause of autoimmune diseases, namely aberrantT cells and/or B cells, which surprisingly translates in vivo to the broad-spectrum activity against autoimmune disease wherein the off-target effects are minimal, in particular liver toxicity.

In comparison to other existing DHDOH inhibitors, farudodstat has high affinity for DHODH, is well tolerated and delivers excellent results to patients. It has the ability to positively impact on patient quality of life to control disease status, and to halt its progression and/or put the disease into remission. Advantageously, healthy cells which have a lower metabolic burden are generally unaffected by the treatment. The balanced characteristics of the treatment are extremely beneficial to patients.

Polymorphs are crystals of the same molecule which have different physical properties as a result of the different organization of molecules within the crystal lattice. Ostwarld stated in 1899, “Almost every substance can exist in two or more solid phases provided the experimental conditions are suitable.”

Differential spatial structure leads to differences in physical properties that can influence various parameters: for example, the compressibility and density, manufacturability, storage, physical stability, chemical stability, thermal stability, melting point, grindability, rheological characteristics of the powder, hygroscopicity, solubility, dissolution rates, in vivo pharmacology, etc.

These properties may in turn may affect parameters relevant to pharmaceuticals, such as the ease of preparing formulations, and/or superior pharmacokinetics. Thus, it is of great importance to select a suitable polymorph in drug development. In particular it is important to identify the dominant stable pharmaceutical polymorph (DSP polymorph). For example, for ranitidine this was form 4.

The present inventors have identified a farudodstat DSP polymorph with excellent stability as well as good pharmacological function.

### SUMMARY OF DISCLOSURE

The present disclosure is summarized in the following paragraphs:

1. A polymorph, such as a crystalline polymorph, of a DHODH inhibitor 2-(3,5-difluoro-3′methoxybiphenyl-4-ylamino)nicotinic acid, characterised by an X-ray powder diffraction pattern comprising the following peaks in terms of degrees of 2-theta at approximately: 9.129±0.1, 17.375±0.1, 20.708±0.1, 25.274±0.1, 26.488±0.1 and 29.041±0.1.

2. The polymorph according to paragraph 1, wherein the X-ray powder diffraction pattern further comprises one or more of the following peaks selected from the group comprising: 13.686±0.1, 14.159±0.1, 19.900±0.1, 21.431±0.1, 22.750±0.1, 25.607±0.1 and 30.893±0.1.

3. The polymorph according to paragraph 2, wherein the pattern comprises a peak at 13.686±0.1, for example with a relative intensity of about 14%.

4. The polymorph according to paragraph 2 or 3, wherein the pattern comprises a peak at 14.159±0.1, for example with a relative intensity of about 16%.

5. The polymorph according to any one of paragraphs 2 to 4, wherein the pattern comprises a peak at 19.900±0.1, for example with a relative intensity of about 11.5%.

6. The polymorph according to any one of paragraphs 2 to 5, wherein the pattern comprises a peak at 21.431±0.1, for example with a relative intensity of about 14%.

7. The polymorph according to any one of paragraphs 2 to 6, wherein the pattern comprises a peak at 22.750±0.1, for example with a relative intensity of about 16.5%.

8. The polymorph according to any one of paragraphs 2 to 7, wherein the pattern comprises a peak at 25.607±0.1, for example with a relative intensity of about 12%.

9. The polymorph according to any one of paragraphs 2 to 8, wherein the pattern comprises a peak at 30.893±0.1, for example with a relative intensity of about 15%.

10. The polymorph according to any one of paragraphs 1 or 9, wherein the X-ray powder diffraction pattern further comprises one or more of the following peaks selected from the group comprising: 12.313±0.1, 13.223±0.1, 16.477±0.1, 18.167±0.1, 22.146±0.1, 23.418±0.1, 23.828±0.1, 25.885±0.1, 27.219±0.1 and 30.612±0.1.

11. The polymorph according to paragraph 10, wherein the X-ray powder diffraction pattern further comprises a peak at 12.313±0.1, for example with a relative intensity of about 10.5%.

12. The polymorph according to paragraph 10 or 11, wherein the X-ray powder diffraction pattern further comprises a peak at 13.223±0.1 for example with a relative intensity of about 7%.

13. The polymorph according to any one of paragraphs 10 to 12, wherein the X-ray powder diffraction pattern further comprises a peak at 16.477±0.1, for example with a relative intensity of about 10.5%.

14. The polymorph according to any one of paragraphs 10 to 13, wherein the X-ray powder diffraction pattern further comprises a peak at 18.167±0.1, for example with a relative intensity of about 7.0%.

15. The polymorph according to any one of paragraphs 10 to 14, wherein the X-ray powder diffraction pattern further comprises a peak at 22.146±0.1, for example with a relative intensity of about 8%.

16. The polymorph according to any one of paragraphs 10 to 15, wherein the X-ray powder diffraction pattern further comprises a peak at 23.418±0.1, for example with a relative intensity of about 10%.

17. The polymorph according to any one of paragraphs 10 to 16, wherein the X-ray powder diffraction pattern further comprises a peak at 25.885±0.1, for example with a relative intensity of about 7.5%.

18. The polymorph according to any one of paragraphs 10 to 17, wherein the X-ray powder diffraction pattern further comprises a peak at 27.219±0.1, for example with a relative intensity of about 9.5%.

19. The polymorph according to any one of paragraphs 10 to 18, wherein the X-ray powder diffraction pattern further comprises a peak at 30.612±0.1, for example with a relative intensity of about 7.5%.

20. The polymorph according to any one of paragraphs 1 to 19, wherein the X-ray powder diffraction pattern further comprises one or more of the following peaks selected from the group comprising: 10.645±0.1, 27.453±0.1, 29.762±0.1, 31.743±0.1 and 39.115±0.1.

21. The polymorph according to paragraph 20, wherein the X-ray powder diffraction pattern further comprises a peak at 10.645±0.1, for example with a relative intensity of about 4.5%.

22. The polymorph according to paragraph 20 or 21, wherein the X-ray powder diffraction pattern further comprises a peak at 27.453±0.1, for example with a relative intensity of about 5.6%.

23. The polymorph according to any one of paragraphs 20 to 22, wherein the X-ray powder diffraction pattern further comprises a peak at 29.762±0.1, for example with a relative intensity of about 4.5%.

24. The polymorph according to any one of paragraphs 20 to 23, wherein the X-ray powder diffraction pattern further comprises a peak at 31.743±0.1, for example with a relative intensity of about 5%.

25. The polymorph according to any one of paragraphs 20 to 23, wherein the X-ray powder diffraction pattern further comprises a peak at 39.115±0.1, for example with a relative intensity of about 6%.

26. The polymorph according to any one of the preceding paragraphs, characterized by an X-ray powder diffraction pattern as shown in FIG. 2.

27. The polymorph according to any one of paragraphs 1 to 26, wherein the mean diameter of particles in the polymorph is in the range 5 to 15 microns, for example 5, 6, 7, 8, 9, 10, 11, 12, 13, 14 or 15.

28. The polymorph according to paragraph 27, wherein 10 percent (D10) of particles in the polymorph have a diameter in the range 0.5 to 5 microns, for example 0.6, 0.7, 0.8, 0.9, 1, 1.5, 2, 2.5, 3, 3.5, 4, 4.5 or 5.

29. The polymorph according to paragraph 27 or 28 wherein 50 percent of particles in the polymorph have a diameter in the range 1 to 10 microns, for example 1, 2, 3, 4, 5, 6, 7, 8, 9 or 10.

30. The polymorph according to any one of paragraphs 27 to 29, wherein 90 percent of particles in the polymorph have a diameter in the range 5 to 50 microns, for example 5, 10, 15, 20, 25, 30, 35, 40, 45 or 50.

31. The polymorph according to any one of paragraphs 1 to 26, wherein the mean diameter of particles in the polymorph is in the 147 to 157 microns, for example 147, 148, 149, 150, 151, 152, 153, 154, 155, 156 or 157.

32. The polymorph according to paragraph 31, wherein 10 percent of particles in the polymorph have a diameter in the range 20 to 40 microns, for example 20, 21, 22, 23, 24, 25, 26, 27, 28, 29, 30, 31, 32, 33, 34, 35, 36, 37, 38, 39 or 40 such as 32 microns.

33. The polymorph according to paragraph 31 or 32 wherein 50 percent of particles in the polymorph have a diameter in the range 150 to 170 microns, for example 150, 151, 152, 153, 154, 155, 156, 157, 158, 159, 160, 161, 162, 163, 164, 165, 166, 167, 168, 169 or 170, such as 161 microns.

34. The polymorph according to any one of paragraphs 31 to 33, wherein 90 percent of particles in the polymorph have a diameter in the range 230 to 245 microns, for example 230, 231, 232, 233, 234, 235, 236, 237, 238, 239, 240, 241, 242, such as 238 microns.

35. A composition comprising the polymorph according to any one of the preceding paragraphs and a pharmaceutically acceptable excipient, diluent or carrier.

36. The polymorph according to any one of paragraphs 1 to 34 or the composition according to paragraph 35 for use in treatment, for example cancer, such as a haematological cancer.

37. The polymorph or composition for use according to paragraph 36, wherein the treatment is for a viral disease or an autoimmune disease.

38. The polymorph or composition for use according to paragraph 37, wherein the virus selected from the group comprising: dengue (such as serotypes 1, 2, 3 or 4), Zika, Chikungunya and SARS-CoV-2.

39. The polymorph or composition for use according to paragraph 37, wherein the autoimmune disease is selected from the group comprising multiple sclerosis, rheumatoid arthritis and inflammatory bowel disease (such as ulcerative colitis and Crohn's disease).

40. The polymorph or composition for use according to paragraph 37, wherein the autoimmune disease is an autoimmune skin disease, for example selected from the group comprising vitiligo, alopecia, atopic dermatitis, Behcet's disease, dermatitis herpetiformis, dermatomyositis, lichen planus, linear IgA disease, lupus of the skin, morphea/scleroderma, ocular cicatrical pemphigoid, pemphigoid such as bullous pemphigoid or pemphigoid gestationis, pemphigus (such as pemphigus vulgaris, pemphigus vulgaris, pemphigus foliaceaous, pemphigus erythematosus, IgA pemphigus, pemphigus vegetans, or paraneoplastic pemphigus), vasculitis, epidermolysis bullosa acquisita, psoriasis, and vesiculobullous dermatosis.

41. The polymorph or composition for use according to paragraph 40, wherein the autoimmune skin disease is selected from the group comprising vitiligo, such as segmented or non-segmented vitiligo, and alopecia, such as diffuse alopecia areata, alopecia areata monolocularis, ophiasis alopecia areata, alopecia areata barbae, alopecia areata totallis or alopecia areata universalis.

42. Use of the polymorph according to any one of paragraphs 1 to 34 or the composition according to paragraph 35 in the manufacture of a medicament for treating a viral infection or an autoimmune disease.

43. The use according to paragraph 42, wherein the virus selected from the group comprising: dengue (such as serotypes 1, 2, 3 or 4), Zika, Chikungunya and SARS-CoV-2.

44. The use according to paragraph 42, wherein the autoimmune disease is selected from the group comprising multiple sclerosis, rheumatoid arthritis and inflammatory bowel disease (such as ulcerative colitis and Crohn's disease).

45. The use according to paragraph 42, wherein the autoimmune disease is an autoimmune skin disease, for example selected from the group comprising vitiligo, alopecia, atopic dermatitis, Behcet's disease, dermatitis herpetiformis, dermatomyositis, lichen planus, linear IgA disease, lupus of the skin, morphea/scleroderma, ocular cicatrical pemphigoid, pemphigoid such as bullous pemphigoid or pemphigoid gestationis, pemphigus (such as pemphigus vulgaris, pemphigus vulgaris, pemphigus foliaceaous, pemphigus erythematosus, IgA pemphigus, pemphigus vegetans, or paraneoplastic pemphigus), vasculitis, epidermolysis bullosa acquisita, psoriasis, and vesiculobullous dermatosis.

46. The use according to paragraph 45, wherein the autoimmune skin disease is selected from the group comprising vitiligo, such as segmented or non-segmented vitiligo, and alopecia, such as diffuse alopecia areata, alopecia areata monolocularis, ophiasis alopecia areata, alopecia areata barbae, alopecia areata totallis or alopecia areata universalis.

47. A method of treating a viral infection or an autoimmune disease comprising administering a therapeutically effective amount of the polymorph according to any one of paragraphs 1 to 34 or a composition according to paragraph 35 to a subject in need thereof.

48. The method according to paragraph 47, wherein the virus selected from the group comprising: dengue (such as serotypes 1, 2, 3 or 4), Zika, Chikungunya and SARS-CoV-2.

49. The method according to paragraph 47, wherein the autoimmune disease is selected from the group comprising multiple sclerosis, rheumatoid arthritis and inflammatory bowel disease (such as ulcerative colitis and Crohn's disease).

50. The method according to paragraph 47, wherein the autoimmune disease is an autoimmune skin disease, for example selected from the group comprising vitiligo, alopecia, atopic dermatitis, Behcet's disease, dermatitis herpetiformis, dermatomyositis, lichen planus, linear IgA disease, lupus of the skin, morphea/scleroderma, ocular cicatrical pemphigoid, pemphigoid such as bullous pemphigoid or pemphigoid gestationis, pemphigus (such as pemphigus vulgaris, pemphigus vulgaris, pemphigus foliaceaous, pemphigus erythematosus, IgA pemphigus, pemphigus vegetans, or paraneoplastic pemphigus), vasculitis, epidermolysis bullosa acquisita, psoriasis, and vesiculobullous dermatosis.

51. The method according to paragraph 50, wherein the autoimmune skin disease is selected from the group comprising vitiligo, such as segmented or non-segmented vitiligo, and alopecia, such as diffuse alopecia areata, alopecia areata monolocularis, ophiasis alopecia areata, alopecia areata barbae, alopecia areata totallis or alopecia areata universalis.

In one embodiment the relative intensity of peak 17.375 is about 100%. In one embodiment the relative intensity of peak 9.129 is about 66%. In one embodiment the relative intensity of peak 20.708 is about 76%. In one embodiment the relative intensity of peak 26.488 is about 43%. In one embodiment the relative intensity of peak 29.041 is about 29%.

Accordingly, in one aspect, there is provided a stable crystalline polymorph of a DHODH inhibitor 2-(3,5-difluoro-3′methoxybiphenyl-4-ylamino)nicotinic acid, characterised by an X-ray powder diffraction pattern comprising the following peaks in terms of degrees of 2-theta at approximately: 9.129±0.1, 17.375±0.1, 20.708±0.1, 25.274±0.1, 26.488±0.1 and 29.041±0.1.

Advantageously, the present inventors have established that the crystalline polymorph of 2-(3,5-difluoro-3′methoxybiphenyl-4-ylamino)nicotinic acid (farudodstat) characterized by the aforementioned X-ray powder diffraction pattern has a very high degree of stability.

In one embodiment the X-ray powder diffraction pattern further comprises one or more of the following peaks selected from the group comprising: 13.686±0.1, 14.159±0.1, 19.900±0.1, 21.431±0.1, 22.750±0.1, 25.607±0.1 and 30.893±0.1.

In one embodiment the X-ray powder diffraction pattern further comprises one or more of the following peaks selected from the group comprising: 12.313±0.1, 13.223±0.1, 18.167±0.1, 22.146±0.1, 23.418±0.1, 23.828±0.1, 25.885±0.1, 27.219±0.1 and 30.612±0.1.

In one embodiment the X-ray powder diffraction pattern further comprises one or more of the following peaks selected from the group comprising: 10.645±0.1, 27.453±0.1, 29.762±0.1, 31.743±0.1 and 39.115±0.1.

In one embodiment the X-ray powder diffraction pattern further comprises one or more of the following peaks selected from the group comprising: 14.580±0.1, 28.368±0.1, 32.950±0.1, 33.445±0.1, 33.800±0.1, 34.936±0.1, 35.497±0.1, 35.981±0.1, 37.678±0.1, 38.099±0.1 and 38.520±0.1.

In one embodiment the X-ray powder diffraction pattern comprises (or further comprises) one or more of the following peaks selected from the group comprising: 13.686±0.1, 14.159±0.1, 19.900±0.1, 21.431±0.1, 22.750±0.1, 25.607±0.1, 30.893±0.1, 12.313±0.1, 13.223±0.1, 18.167±0.1, 22.146±0.1, 23.418±0.1, 23.828±0.1, 25.885±0.1, 27.219±0.1 and 30.612±0.1.

In one embodiment the X-ray powder diffraction pattern comprises (or further comprises) one or more of the following peaks selected from the group comprising: 13.686±0.1, 14.159±0.1, 19.900±0.1, 21.431±0.1, 22.750±0.1, 25.607±0.1, 30.893±0.1, 12.313±0.1, 13.223±0.1, 18.167±0.1, 22.146±0.1, 23.418±0.1, 23.828±0.1, 25.885±0.1, 27.219±0.1, 30.612±0.1, 10.645±0.1, 27.453±0.1, 29.762±0.1, 31.743±0.1 and 39.115±0.1.

In one embodiment the X-ray powder diffraction pattern further comprises one or more of the following peaks selected from the group comprising: 13.686±0.1, 14.159±0.1, 19.900±0.1, 21.431±0.1, 22.750±0.1, 25.607±0.1, 30.893±0.1, 12.313±0.1, 13.223±0.1, 18.167±0.1, 22.146±0.1, 23.418±0.1, 23.828±0.1, 25.885±±0.1, 27.219±0.1, 30.612±0.1, 10.645±0.1, 27.453±0.1, 29.762±0.1, 31.7430.1, 39.115±0.1, 14.5800.1, 28.368±0.1, 32.950±0.1, 33.4450.1, 33.800±0.1, 34.936±0.1, 35.497±0.1, 35.981±0.1, 37.678±0.1, 38.099±0.1 and 38.520±0.1.

In one embodiment the X-ray powder diffraction pattern further comprises the following peaks: 13.686±0.1, 14.159±0.1, 19.900±0.1, 21.431±0.1, 22.750±0.1, 25.607±0.1, 30.893±0.1, 12.313±0.1, 13.223±0.1, 18.167±0.1, 22.146±0.1, 23.418±0.1, 23.828±0.1, 25.885±0.1, 27.219±0.1 and 30.612±0.1.

In one embodiment the X-ray powder diffraction pattern further comprises the following peaks: 13.686±0.1, 14.159±0.1, 19.900±0.1, 21.431±0.1, 22.750±0.1, 25.607±0.1, 30.893±0.1, 12.313±0.1, 13.223±0.1, 18.167±0.1, 22.146±0.1, 23.418±0.1, 23.828±0.1, 25.885±0.1, 27.219±0.1, 30.612±0.1, 10.645±0.1, 27.453±0.1, 29.762±0.1, 31.743±0.1 and 39.115±0.1.

In one embodiment the X-ray powder diffraction pattern further comprises the following peaks: 13.686±0.1, 14.159±0.1, 19.900±0.1, 21.431±0.1, 22.750±0.1, 25.607±0.1, 30.893±0.1, 12.313±0.1, 13.223±0.1, 18.167±0.1, 22.146±0.1, 23.418±0.1, 23.828±0.1, 25.885±0.1, 27.219±0.1, 30.612±0.1, 10.645±0.1, 27.453±0.1, 29.762±0.1, 31.743±0.1, 39.115±0.1, 14.580±0.1, 28.368±0.1, 32.950±0.1, 33.445±0.1, 33.800±0.1, 34.936±0.1, 35.497±0.1, 35.981±0.1, 37.678±0.1, 38.099±0.1 and 38.520±0.1.

In one embodiment the X-ray powder diffraction pattern comprises the following peaks: 9.129±0.1, 10.645±0.1, 12.313±0.1, 13.223±0.1, 13.686±0.1, 14.159±0.1, 14.580±0.1, 16.477±0.1, 17.375±0.1, 18.167±0.1, 19.900±0.1, 20.708±0.1, 21.431±0.1, 22.146±0.1, 22.750±0.1, 23.418±0.1, 23.828±0.1, 25.274±0.1, 25.607±0.1, 25.885±0.1, 26.488±0.1, 27.219±0.1, 27.453±0.1, 28.368±0.1, 29.041±0.1, 29.762±0.1, 30.612±0.1, 30.893±0.1, 31.743±0.1, 32.950±0.1, 33.445±0.1, 33.800±0.1, 34.936±0.1, 35.497±0.1, 35.981±0.1, 37.678±0.1, 38.099±0.1, 38.520±0.1 and 39.115±0.1.

In one embodiment the polymorph is characterized by an X-ray powder diffraction (XRPD) pattern as shown in FIG. 2. In one embodiment the polymorph is characterized by an XRPD pattern as shown as Pattern 1 in FIG. 26.

In one embodiment the polymorph is characterized by an XRPD pattern comprising one or more (1, 2, 3, 4, 5, 6, 7, 8, 9, 10, 11, 12, 13, 14, 15, 16, 17, 18, 19, 20, 21, 22, 23, 24, 25, 25 27, 28, 29, 30, 31, 32, 33, 34, 35, 36, 37, 38 or all) peaks as listed in FIG. 3 (which shows peaks 1 to 39). In one embodiment the polymorph is characterized by an XRPD pattern comprising all the peaks as listed in FIG. 3.

In one aspect there is provided a polymorph, such as a crystalline polymorph, of a DHODH inhibitor 2-(3,5-difluoro-3′methoxybiphenyl-4-ylamino)nicotinic acid, characterised by an X-ray powder diffraction pattern comprising one or more of the following peaks in terms of degrees of 2-theta at approximately: 8.9072±0.1, 10.464±0.1, 12.132±0.1, 13.5505±0.1, 13.9897±0.1, 16.3257±0.1, 17.0936±0.1, 17.2922±0.1, 19.6989±0.1, 20.4996±0.1, 21.225±0.1, 22.0126±0.1, 22.5466±0.1, 22.7501±0.1, 25.121±0.1, 26.3694±0.1, 27.2907±0.1, 28.7305±0.1, 29.3309±0.1, 30.7531±0.1, 31.7095±0.1, 32.9909±0.1, and 34.266±0.1.

In one embodiment the polymorph comprises one or more of the following peaks: 8.9072±0.1, 17.0936±0.1, 20.4996±0.1, 25.121±0.1, and 26.3694±0.1. In one embodiment, the polymorph comprises at least the following peaks: 8.9072±0.1, 17.0936±0.1, 20.4996±0.1, 25.121±0.1, and 26.3694±0.1.

In one embodiment the polymorph is characterized by an XRPD pattern comprising the following peaks: 8.9072±0.1, 10.464±0.1, 12.132±0.1, 13.5505±0.1, 13.9897±0.1, 16.3257±0.1, 17.0936±0.1, 17.2922±0.1, 19.6989±0.1, 20.4996±0.1, 21.225±0.1, 22.0126±0.1, 22.5466±0.1, 22.7501±0.1, 25.121±0.1, 26.3694±0.1, 27.2907±0.1, 28.7305±0.1, 29.3309±0.1, 30.7531±0.1, 31.7095±0.1, 32.9909±0.1, and 34.266±0.1.

In one embodiment the polymorph is characterized by an XRPD pattern as shown as Pattern 1 in FIG. 26.

In one embodiment the polymorph is characterized by an XRPD pattern comprising one or more (1, 2, 3, 4, 5, 6, 7, 8, 9, 10, 11, 12, 13, 14, 15, 16, 17, 18, 19, 20, 21, 22, 23, 24, 25, 25 27, 28, 29, or all peaks as listed in FIG. 43 (which shows peaks 1 to 30). In one embodiment the polymorph is characterized by an XRPD pattern comprising all the peaks listed in FIG. 43.

In one embodiment the polymorph is characterized by an XRPD pattern comprising one or more of peaks 1, 2, 3, 4, 5, 6, 7, 8, 12, 13, 14, 15, 16, 17, 20, 22, 23, 24, 25, 27, 28, 29 and 30 as listed in FIG. 43. In one embodiment the polymorph is characterized by an XRPD pattern comprising peaks 1, 2, 3, 4, 5, 6, 7, 8, 12, 13, 14, 15, 16, 17, 20, 22, 23, 24, 25, 27, 28, 29 and 30 as listed in FIG. 43.

In an independent aspect, there is provided a polymorph, such as a crystalline polymorph of a DHODH inhibitor 2-(3,5-difluoro-3′methoxybiphenyl-4-ylamino)nicotinic acid, characterised by an X-ray powder diffraction pattern comprising one or more of the following peaks in terms of degrees of 2-theta at approximately: 9.3112±0.1, 10.2275±0.1, 11.2939±0.1, 11.9453±0.1, 14.3786±0.1, 14.7466±0.1, 16.1054±0.1, 16.561±0.1, 17.0456±0.1, 17.6267±0.1, 18.2131±0.1, 18.6757±0.1, 20.5363±0.1, 22.7337±0.1, 23.5524±0.1, 24.0002±0.1, 24.4047±0.1, 24.8923±0.1, 25.2252±0.1, 25.8169±0.1, 27.3981±0.1, 28.1985±0.1, 30.8567±0.1, and 32.9406±0.1.

In one embodiment, there is provided a polymorph, such as a crystalline polymorph of a DHODH inhibitor 2-(3,5-difluoro-3′methoxybiphenyl-4-ylamino)nicotinic acid, characterised by an X-ray powder diffraction pattern comprising the following peaks in terms of degrees of 2-theta at approximately: 9.3112±0.1, 10.2275±0.1, 11.2939±0.1, 11.9453±0.1, 14.3786±0.1, 14.7466±0.1, 16.1054±0.1, 16.561±0.1, 17.0456±0.1, 17.6267±0.1, 18.2131±0.1, 18.6757±0.1, 20.5363±0.1, 22.7337±0.1, 23.5524±0.1, 24.0002±0.1, 24.4047±0.1, 24.8923±0.1, 25.2252±0.1, 25.8169±0.1, 27.3981±0.1, 28.1985±0.1, 30.8567±0.1, and 32.9406±0.1.

In an independent aspect, there is provided a polymorph, such as a crystalline polymorph of a DHODH inhibitor 2-(3,5-difluoro-3′methoxybiphenyl-4-ylamino)nicotinic acid, characterised by an X-ray powder diffraction pattern as shown as Pattern 2 in FIG. 37.

In one embodiment the polymorph is characterized by an XRPD pattern comprising one or more (1, 2, 3, 4, 5, 6, 7, 8, 9, 10, 11, 12, 13, 14, 15, 16, 17, 18, 19, 20, 21, 22, 23 or all) peaks as listed in FIG. 38. In one embodiment the polymorph is characterized by an XRPD pattern comprising all the peaks listed in FIG. 38.

In one embodiment the solid form of the present disclosure is a solvate, for example a hydrate or hemihydrate.

In one embodiment the polymorph is a crystalline polymorph. In one embodiment the particles are an irregular shape. In one embodiment the particles are a regular shape, spherical.

In one embodiment the compound (API) is milled. Examples of milling include cone milling, hammer milling.

In one embodiment the compound (API) is micronized, for example to render the particle size uniform, such as a D90 of 10 microns or less.

Primary particle size is influence by temperature. In one embodiment the primary particle size is developed at 15-65 degrees Celsius, for example 15, 20, 25, 30, 35, 40, 45, 50, 55, 60 or 65 degrees Celsius. In one embodiment the primary particle size is developed at 20-60 degrees Celsius, such as 20, 25, 30, 35, 40, 45, 50, 55 or 60 degrees Celsius. In one embodiment the primary particle size is developed at 20-35 degrees Celsius, such as 20, 21, 22, 23, 24, 25, 26, 27, 28, 29, 30, 31, 32, 33, 34, 35 degrees Celsius.

Solvent also influences the primary particle size. In one embodiment the solvent is one described in the examples herein or a combination of the same.

### DETAILED DESCRIPTION

Dominant stable pharmaceutical polymorph as employed herein refers a polymorph form that once prepared becomes the dominant form (for example superseding other previous forms) and is suitable for use as a pharmaceutical API (active product ingredient), in particular the polymorph described in the Examples and Figures herein.

2-(3, 5-difluoro-3′-methoxybiphenyl-4-ylamino) nicotinic acid (referred to herein as farudodstat or ASLAN003) has the structure:

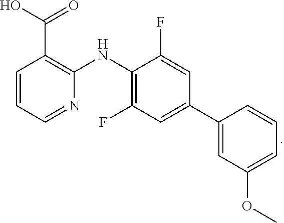 [SMILES: COc1cccc(-c2cc(F)c(Nc3ncccc3C(=O)O)c(F)c2)c1]

The term “polymorph” as used herein refers to a particular crystal form of a chemical compound that can exist in more than one crystal form in the solid state. A crystal form of a compound contains the constituent molecules arranged in orderly repeating patterns extending in all three spatial dimensions (in contrast, an amorphous solid form has no long-range order in the position of molecules). Different polymorphs of a compound have different arrangements of atoms and or molecules in their crystal structure. When the compound is a biologically active compound, the difference in crystal structures can lead to different polymorphs having differing chemical, physical and biological properties. Properties which may be affected include crystal shape, density, hardness, colour, chemical stability, melting point, hygroscopicity, suspensibility, dissolution rate and biological availability. As such, a specific polymorph may have properties which make it more advantageous relative to another polymorph of the same compound. Note that predicting whether the solid state of a compound may be present as more than one polymorph is not possible and nor is it possible to predict the properties of any of these crystal forms.

X-ray powder diffraction (XRPD) is a rapid analytical technique primarily used for phase identification of a crystalline material and can provide information on unit cell dimensions. The analysed material is finely ground, homogenized, and average hulk composition is determined. X-ray diffractometers generate X-rays in an x-ray tube and direct the X-rays at a sample. As the sample and detector are rotated, the intensity of the reflected X-rays is recorded. When the geometry of the incident X-rays impinging the sample satisfies the Bragg Equation, constructive interference occurs and a peak in intensity occurs. A detector records and processes this X-ray signal and converts the signal to a count rate which is then output to a device such as a printer or computer monitor. The geometry of an X-ray diffractometer is such that the sample rotates in the path of the collimated X-ray beam at an angle θ while the X-ray detector is mounted on an arm to collect the diffracted X-rays and rotates at an angle of 2θ (two theta). The data is collected is used to generate an X-ray powder diffraction pattern.

The term “X-ray powder diffraction pattern” as used herein refers to a plot of intensity on the y-axis against the angle of the detector 2θ (two theta) on the x-axis. X-ray powder diffraction pattern peak positions are generally reported for polymorphs in terms of the angular positions (two theta) with an allowable variability of 0.1, which is specified by the U.S. pharmacopeia, pages 1843-1844 (1995).

The variability of ±0.1 is intended to be used when comparing two powder X-ray diffraction patterns. In practice, if a diffraction pattern peak from one pattern is assigned a range of angular positions (two theta) which is the measured peak position ±0.1 and a diffraction pattern peak from the other pattern is assigned a range of angular positions (two theta) which is the measured peak position ±0.1 and if those ranges of peak positions overlap, then the two peaks are considered to have the same angular position (two theta).

The skilled person is aware that a given polymorph analysed by different instruments, laboratories, at different temperatures etc can generate spectra with a certain amount of variation. However, the profile and proportions of the spectra for the same polymorph will be identifiable. Thus a shift or drift in a spectra would not normally hinder the skilled person positively identifying the presence of the given polymorph. Thus in one embodiment an X-Ray diffusion patterns is shifted by up to and including 10% along the X axis, for example 1, 2, 3, 4, 5, 6, 7, 8, 9 or 10%.

Drift is a measurement error caused by the gradual shift in a gauge's measured values over time. Although incorrect handling can accelerate it, nearly all measuring instruments will experience drift during their lifetime.

In one embodiment the farudodstat polymorph of the present disclosure is produced using the following manufacturing process (also shown in FIG. 1A):

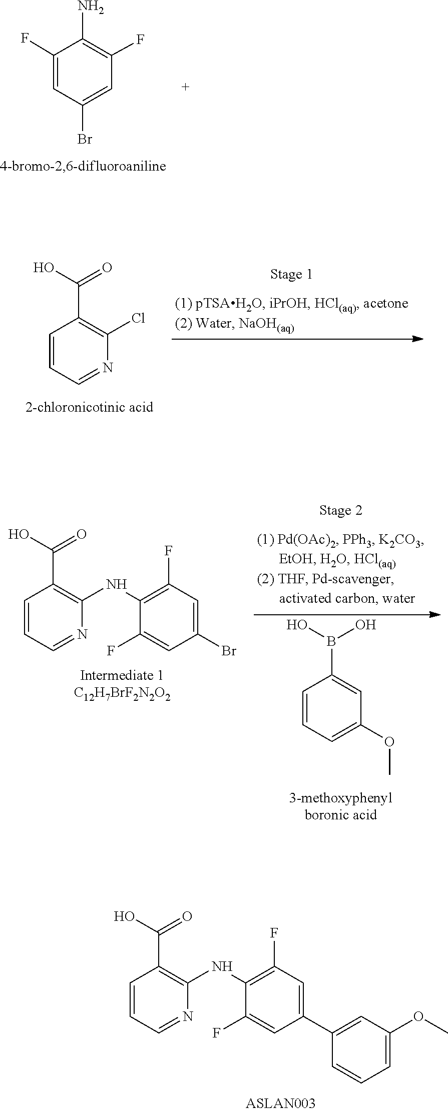 [SMILES: Nc1c(F)cc(Br)cc1F ; O=C(O)c1cccnc1Cl ; O=C(O)c1cccnc1Nc1c(F)cc(Br)cc1F ; COc1cccc(B(O)O)c1 ; COc1cccc(-c2cc(F)c(Nc3ncccc3C(=O)O)c(F)c2)c1]

### Formulations

A DHODH inhibitor is a moiety (such as a compound) that inhibits, for example reduces or blocks the activity of a DHODH enzyme (see background for definition thereof).

In one embodiment the DHODH inhibitor is provided as a pharmaceutical formulation.

The pharmaceutical compositions of this invention may be administered by any number of routes including, but not limited to, oral, intravenous, intramuscular, intra-arterial, intramedullary, intrathecal, intraventricular, transdermal, transcutaneous (e.g. WO98/20734), subcutaneous, intraperitoneal, intranasal, enteral, topical, sublingual, intravaginal or rectal routes.

In one embodiment the pharmaceutical formulation is for oral administration, for example formulated as tablets, pills, dragees, capsules, liquids, gels, syrups, slurries and suspensions, for ingestion by the patient.

Excipients may include lactose, dextrin, glucose, sucrose, sorbitol, starch, sugars, sugar alcohols and cellulose.

Other suitable forms for administration include parenteral administration, for example injection or infusion, such as bolus injection or continuous infusion.

Where the product is for injection or infusion, it may take the form of a suspension, solution or emulsion in an oily or aqueous vehicle and it may contain formulatory agents, such as a suspending agent, preservative, stabilising and/or dispersing agents. Alternatively, the molecule may be in dry form, for reconstitution before use with an appropriate sterile liquid. Pharmaceutically acceptable carriers in therapeutic compositions may additionally contain liquids such as water, saline, glycerol and ethanol. Additionally, auxiliary substances, such as wetting agent, emulsifying agents, lubricant or pH buffering substances, may be present in such compositions. A thorough discussion of pharmaceutically acceptable carriers is available in Remington's Pharmaceutical Sciences (Mack Publishing Company, N.J. 1991).

### Treatment

Treatment as employed herein refers to where the patient has a disease or disorder, for example autoimmune disease (in particular one disclosed herein) and the medicament according to the present disclosure is administered to stabilise the disease, delay the disease, ameliorate the disease, send the disease into remission, maintain the disease in remission or cure the disease. Treating as employed herein includes administration of a medicament according to the present disclosure for treatment or prophylaxis.

Treatment or therapy may be employed prophylactically.

Therapeutically effective amount as employed herein is an amount in the range which generates a desirable physiological effect, whilst minimising side effects.

Disease modifying therapy as employed herein refers to therapy that allows the immune system to reset itself and rebalance, thereby performing more normally after treatment.

The DHODH inhibitor of the disclosure or formulation comprising the same may be administered at a dose in the range of 1 mg to 400 mg per day, such as 10 mg to 400 mg per day, 50 mg to 400 mg per day, 100 mg to 400 mg per day, 150 mg to 400 mg per day, 200 mg to 400 mg per day, 250 mg to 400 mg per day, 300 mg to 400 mg per day, or 350 mg to 400 mg per day.

In particular, a dose in the range of 100 mg to 400 mg per day is administered. Thus, the daily dose may be for example 10 mg, 20 mg, 30 mg, 40 mg, 50 mg, 60 mg, 70 mg, 80 mg, 90 mg, 100 mg, 110 mg 120 mg, 130 mg, 140 mg, 150 mg 160 mg, 170 mg, 180 mg, 190 mg, 200 mg, 210 mg, 220 mg, 230 mg, 240 mg, 250 mg, 260 mg, 270 mg, 280 mg, 290 mg 300 mg, 310 mg, 320 mg, 330 mg, 340 mg 350 mg, 360 mg, 370 mg 380 mg, 390 mg or 400 mg.

In one embodiment the treatment is administered daily, for example once or twice daily. In one embodiment the treatment is once daily.

In one embodiment farudodstat is administered orally, for example as a tablet or capsule or caplet. Co-morbidity as employed herein refers to where the patient is suffering from a second or underlying health condition.

Combination therapy (comprising further therapy) as employed herein wherein two or more treatment regimens are employed, in particularly employed concomitantly. The treatments may be separate formulations or co-formulated. They may be administered at the same time or different times. However, the pharmacological effect of the treatments will co-exist in the patient.

Further therapy as employed herein refers to a therapy in addition to the DHODH inhibitor. Such a further therapy may be an anti-inflammatory agent, which includes but is not limited to, a non-steroidal anti-inflammatory agent (NSAID), a disease modifying anti-rheumatic drug (DMARD), a statin (including HMG-CoA reductase inhibitors such as simvastatin), a biological agent (biologicals), a steroid, an immunosuppressive agent, a salicylate and/or a microbicidal agent.

Non-steroidal anti-inflammatory agents include anti-metabolite agents (such as methotrexate) and anti-inflammatory gold agents (including gold sodium thiomalate, aurothiomalate or gold salts, such as auranofin). Biologicals include anti-TNF agents (including adalimumab, etanercept, infliximab, anti-IL-1 reagents, anti-IL-6 reagents, anti-CD20 agents, anti-B cell reagents (such as rituximab), anti-T cell reagents (anti-CD4 antibodies), anti-IL-15 reagents, anti-CLTA4 reagents, anti-RAGE reagents), antibodies, soluble receptors, receptor binding proteins, cytokine binding proteins, mutant proteins with altered or attenuated functions, RNAi, polynucleotide aptamers, antisense oligonucleotides or omega 3 fatty acids. Steroids (also known as corticosteroids) include cortisone, prednisolone or dexamethasone may also be employed in a combination therapy.

Immunosuppressive agents for use in a combination therapy according to the present disclosure include cyclosporin, FK506, rapamycin, mycophenolic acid. Salicylates for use in said combination therapy include aspirin, sodium salicylate, choline salicylate and magnesium salicylate. Microbicidal agents include quinine and chloroquine.

Anti-inflammatory as employed herein refers to a moiety that reduced inflammation, for a non-steroidal anti-inflammatory, steroids and the like.

In one embodiment, the combination therapy comprises an anti-CD20 agent or a biosimilar thereof, for example Rituxan (rituximab), a Rituximab biosimilar, Gazyva, Kesimpta, Ocrevus (ocrelizumab), Ruxience, Truxima, Zevalin, Arzerra, AcellBia, HLX01, Reditux, Ritucad or Zytux.

In one embodiment, the combination therapy comprises a treatment independently selected from corticosteroids (for example oral prednisone and intravenous methylprednisolone), plasma exchange (plasmapheresis), interferon beta medications, glatiramer acetate, fingolimod, dimethyl fumarate, diroximel fumarate, teriflunomide, siponimod, cladribine, ocrelizumab, natalizumab and alemtuzumab.

In one embodiment, the combination therapy comprises a treatment to ease or reduce the symptoms of multiple sclerosis, for example a muscle relaxant (such as baclofen, tizanidine and cyclobenzaprine), a medication to reduce fatigue (such as amantadine, modafinil, methylphenidate, or a medication to increase walking speed (such as dalfampridine).

In one embodiment the combination therapy comprises cannabis or a derivative thereof, for example cannabis oil.

In one embodiment the combination therapy comprises a second DHODH inhibitor. In one embodiment the further therapy comprises teriflunomide. In one embodiment the further therapy comprises vidofludimus. In one embodiment the combination therapy does not comprise a second DHODH inhibitor. In embodiment the combination therapy does not comprise teriflunomide and/or vidofludimus.

In one embodiment the combination therapy comprises a disease modifying therapy, for example selected from alemtuzumab, avonex, betaferon, cladribine, daclizumab, dimethyl fumerate, extavia, fingolimod, glatiramer acetate, natalizumab, ocrelizumab, plegridy, rebif, siponimod and combinations of two or more of the same.

In one embodiment, the further therapy comprises interferon beta (IFN-β), such as interferon beta-1a or interferon beta-1b. Hence, in one embodiment the further therapy comprises interferon beta-1a. In an alternative embodiment, the further therapy comprises interferon beta-1b.

In one embodiment, the combination therapy comprises a Bruton's Tyrosine Kinase (BTK) inhibitor, for example Ibrutinib, Acalabrutinib, Zanubrutinib, Evobrutinib, ABBV-105, Fenebrutinib, GS-4059, Spebrutinib and/or HM71224.

In one embodiment, the further therapy comprises glatiramer acetate. In one embodiment the further therapy comprises natalizumab. In one embodiment, the further therapy comprises mitoxantrone. In one embodiment, the further therapy comprises fingolimod. In one embodiment the further therapy comprises Siponimod. In one embodiment, the further therapy comprises dimethyl fumarate. In one embodiment the further therapy comprises alemtuzumab. In one embodiment, the further therapy comprises cyclophosphamide. In one embodiment, the further therapy comprises cladribine. In one embodiment the further therapy comprises ocrelizumab. In one embodiment, the further therapy comprises dimethyl fumarate. In one embodiment, the further therapy comprises daclizumab. In on embodiment the further therapy comprises azathioprine. In one embodiment, the further therapy comprises methotrexate. In an alternative embodiment, the further therapy does not comprise methotrexate. In one embodiment the further therapy comprises lacquinimod.

### Autoimmune Disease

Autoimmune disease as used herein refers to any disease or condition wherein an individual's immune system mistakenly targets that individual's own normal “healthy” cells, in particular characterised by aberrant T cell and/or B cell activation.

Aberrant T cell and/or B cell activation as employed herein refers to abnormal T cell and/or B cell activation, in particular where the abnormal cells recognise self or self antigens.

Severe autoimmune disease is where the disease is not controlled by standard of care medicaments/treatments. Flare, is a period of disease exacerbation.

“Inadequate control” as employed herein refers to where standard of care medication fails to lessen or control symptoms, in particular where the patient's quality of life is adversely affected.

“Defined endpoint” as employed herein refers to clinically defined point, for example remission or stable disease.

In one embodiment farudodstat is employed in maintenance therapy, for example at a low dose. Maintenance therapy as employed herein refers to continuous therapy to make the disease stable or to keep the disease in remission, for example where the dose administered is low and in particular frequent. A dose of 100 to 200 mg for example given once or twice a day may be used as maintenance therapy.

In one embodiment the autoimmune disease is an autoimmune skin disease i.e. autoimmune disease manifested or presented in the skin in particular the dermis and/or epidermis, such as lupus. In one embodiment the autoimmune skin disease is selected from the group comprising: vitiligo, alopecia, Behcet's disease, dermatitis herpetiformis, dermatomyositis, lichen planus, linear IgA dermatosis, lupus of the skin, morphea/scleroderma, ocular cicatrical pemphigoid, pemphigoid such as bullous pemphigoid or pemphigoid gestationis, pemphigus (such as pemphigus vulgaris, pemphigus vulgaris, pemphigus foliaceaous, pemphigus erythematosus, IgA pemphigus, pemphigus vegetans, or paraneoplastic pemphigus), vasculitis, epidermolysis bullosa acquisita, psoriasis, vesiculobullous dermatosis and atopic dermatitis.

In one embodiment the autoimmune skin disease is selected from the group comprising vitiligo and alopecia. In one embodiment the autoimmune skin disease is vitiligo and alopecia.

In one embodiment the autoimmune skin disease is vitiligo. In one embodiment the autoimmune skin disease is vitiligo is selected from the group comprising segmental vitiligo or non-segmental vitiligo. In one embodiment the vitiligo is segmental vitiligo. In one embodiment the vitiligo is non-segmental vitiligo (for example focal vitiligo, generalised vitiligo or acrofacial vitiligo).

In one embodiment the autoimmune skin disease is alopecia. In one embodiment the alopecia is selected from the group comprising diffuse alopecia areata, alopecia areata monolocularis, ophiasis alopecia areata, alopecia areata barbae, alopecia areata totallis or alopecia areata universalis. In one embodiment the alopecia is diffuse alopecia areata. In one embodiment the alopecia is alopecia areata monolocularis. In one embodiment the alopecia is ophiasis alopecia areata. In one embodiment the alopecia is alopecia areata barbae. In one embodiment the alopecia is alopecia areata totallis. In one embodiment the alopecia is alopecia areata universalis.

Thus, in one embodiment the autoimmune disease is selected from the group comprising or consisting of Acute disseminated encephalomyelitis (adem), acute necrotizing hemorrhagic leukoencephalitis, Addison's disease, adrenal insufficiency, hypocortisolism, amyloidosis, ankylosing spondylitis, spondyloarthritis, Strumpell-marie disease, anti-GBM/anti-TBM nephritis, antiphospholipid syndrome (aps), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune lymphoproliferative syndrome (ALPS), Canale-Smith syndrome, autoimmune myocarditis, autoimmune oophoritis, autoimmune pancreatitis (AIP), autoimmune polyglandular syndromes(types I, II & III), autoimmune retinopathy (AR), autoimmune thrombocytopenic purpura (ATP), autoimmune thyroid disease, autoimmune urticaria, axonal/neuronal neuropathies, balo disease, Behcet's disease, bullous pemphigoid, cardiomyopathy, Castleman disease, coeliac disease, chagas disease, chronic inflammatory demyelinating polyneuropathy (CIDP), chronic recurrent multifocal ostomyelitis (CRMO), Churg-Strauss syndrome, cicatricial pemphigoid/benign mucosal pemphigoid (CP), Crohn's disease, inflammatory bowel disease, colitis (including ulcerative colitis), enteritis, ileitis, Cogans syndrome, cold agglutinin disease, congenital heart block, Coxsackie myocarditis, crest disease, cryoglobulinemia, demyelinating neuropathies, dermatitis herpetiformis, Duhring's disease, dermatomyositis, diabetes, type I, discoid lupus erythematosus (DLE), Dressler's syndrome, endometriosis, epidermolysis bullosa (EB) and eb acquisita (EBA), eosinophilic gastroenteritis, esophagitis, eosinophilic fasciitis, schulman's syndrome, erythema nodosum, experimental allergic encephalomyelitis, Evans syndrome, fibrosing alveolitis, giant cell arteritis (temporal arteritis), giant cell myocarditis, glomerulonephritis (non-proliferative: focal segmental glomerulosclerosis and membranous glomerulonephritis. proliferative: IgA nephropathy), goodpasture's syndrome, granulomatosis with polyangiitis (GPA) (formerly called Wegener's granulomatosis), Graves' disease, Guillain-Barre syndrome, Miller Fisher syndrome, acute motor axonal neuropathy, acute motor sensory axonal neuropathy, acute panautonomic neuropathy, Bickerstaff's brainstem encephalitis, Hashimoto's encephalitis, Hashimoto's thyroiditis, hemolytic anemia, Henoch-Schonlein purpura, herpes gestationis, hypogammaglobulinemia, idiopathic pulmonary fibrosis, idiopathic thrombocytopenic purpura (ITP), IgA nephropathy (IGAN), berger's syndrome, synpharyngitic glomerulonephritis, IgA pemphigus, IgG4-related sclerosing disease, immune-regulated infertility, inclusion body myositis, insulin-dependent diabetes mellitus, interstitial cystitis, Isaac's syndrome, neuromyotonia, juvenile arthritis, juvenile myositis, Kawasaki syndrome, Lambert-Eaton syndrome, leukocytoclastic vasculitis, lichen planus, lichen sclerosus, ligneous conjunctivitis, linear IgA dermatosis (LAD), pemphigoid, lupus (SLE), lyme disease, Meniere's disease, microscopic polyangiitis (MPA), mixed connective tissue disease (MCTD), monoclonal gammaopathy, Mooren's ulcer, Mucha-Habermann disease, multiple sclerosis, myasthenia gravis, myositis, narcolepsy, neuromyelitis optica (devic's), neuromyotonia, Isaac's syndrome (acquired, paraneoplastic, hereditary), neutropenia, ocular cicatricial pemphigoid, optic neuritis, oophoritis, opsoclonus-myoclonus syndrome, orchitis, palindromic rheumatism, pandas (pediatric autoimmune neuropsychiatric disorders associated with streptococcus), paraneoplastic autoimmune multiorgan syndrome (PAMS), paraneoplastic cerebellar degeneration, paraneoplastic pemphigus (PNP), paroxysmal nocturnal hemoglobinuria (PNH), Parry Romberg syndrome, Parsonnage-Turner syndrome, pars planitis (peripheral uveitis), pempgigoid gestationis (PG), pemphigus vulgaris (PV), pemphigus folliaceus (PF), peripheral neuropathy, perivenous encephalomyelitis, pernicious anemia, Poems syndrome, polyarteritis nodosa (PAN), polymyalgia rheumatic, polymyositis, postmyocardial infarction syndrome, postpericardiotomy syndrome, progesterone dermatitis primary biliary cirrhosis, Hanot syndrome, primary sclerosing cholangitis (PSC), sclerosong cholangitis, psoriasis, psoriatic arthritis, pyoderma gangrenosum, pure red cell aplasia, Rasmussen's encephalitis, chronic focal encephalitis (CFE), Raynauds phenomenon, reactive arthritis, Reiter's syndrome, recoverin-associated retinopathy (RAR), reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, systemic sclerosis, Sjogren's syndrome, sperm & testicular autoimmunity, stiff person/man syndrome, subacute bacterial endocarditis (SBE), Susac's syndrome, sympathetic ophthalmia, Takayasu's arteritis, temporal arteritis/giant cell arteritis, thromboangiitis obliterans, Buerger's disease, thrombocytopenic purpura (TTP), Tolosa-Hunt syndrome, transverse myelitis, undifferentiated connective tissue disease (UCTD), uveitis, Takayasu's arteritis, temporal arteritis, Buerger's disease, cutaneous vasculitis, Kawasaki disease, polyarteritis nodosa, Behçet's syndrome, Churg-Strauss syndrome, cutaneous vasculitis, Henoch-Schönlein purpura, microscopic polyangiitis, Wegener's granulomatosis, golfer's vasculitis, vesiculobullous dermatosis, and wegener's granulomatosis (now termed granulomatosis with polyangiitis (GPA).

In one embodiment the autoimmune disease is selected from the group comprising or consisting of ANCA vasculitis, IgA nephropathy (Berger's), pemphigus vulgaris/bullous pemphigoid, ITP, primary biliary cirrhosis, autoimmune thyroiditis (Grave's disease), hashimoto's disease, lupus nephritis, membranous glomerulonephritis (or membranous nephropathy), APS, myasthenia gravis, neuromyelitis optica, primary Sjogren's, autoimmune neutropaenia, autoimmune pancreatitis, dermatosmyositis, autoimmune uveitis, autoimmune retinopathy, Behçet's disease, IPF, systemic sclerosis, liver fibrosis, autoimmune hepatitis, primary sclerosing cholangitis, goodpasture's syndrome, pulmonary alveolar proteinosis, chronic autoimmune urticarial, psoriasis, rheumatoid arthritis, psoriatic arthritis, axial spodyloarthritis, transplantation (including GvHD), asthma, COPD, giant cell arteritis, refractory autoimmune cytopaenias, Evans syndrome (autoimmune haemolytic anaemia), type I diabetes, sarcoidosis, polymyositis, ulcerative colitis, Crohn's disease, coeliac disease, Waldenstrom's macroglobulinaemia, focal segmental glomerulosclerosis, chronic Lyme disease (Lyme borreliosis), lichen planus, Stiff person syndrome, dilated cardiomyopathy, autoimmune (lymphocytic) oophoritis, epidermolysis bullosa acquisita, autoimmune atrophic gastritis, pernicious anaemia, atopic dermatitis, atherosclerosis, multiple sclerosis, Rasmussen's encephalitis, Guillain-Barré syndrome, acquired neuromyotonia, and stroke.

In one embodiment, the autoimmune disease is selected from the group comprising or consisting of Hidradenitis suppurativa, Scleroderma (Systemic scleritis), Lichen planus, Morphea, Psoriasis, Diabetes mellitus type 1, Autoimmune thyroiditis, Graves' disease, Endometriosis, Coeliac disease, Crohn's disease, Ulcerative colitis, Axial spondylitis, Juvenile arthritis, Palindromic rheumatism, Psoriatic arthritis, Rheumatoid arthritis, Sarcoidosis, Systemic lupus erythematosus (SLE), Undifferentiated connective tissue disease (UCTD), Multiple sclerosis, pattern II, Restless legs syndrome, Optic neuritis, Uveitis, Scleritis, Mooren's ulcer, Meniere's disease, Graves' opthalmopathy, Neuromyelitis optica, Susac's syndrome, and lupus erythrematosus.

Autoimmune skin disease or disorder as used herein refers to any disease or condition wherein an individual's immune system, for example the individual's T cells and/B cells, mistakenly targets that individual's skin tissue or cells therein, including hair follicles. Farudodstat is able to block the biosynthesis of pyrimidine, which is thought to cause cell cycle arrest leading to a reduction in pro-inflammatory cytokine production and/or apoptosis in T cells and/or B cells. Thus, farudodstat is believed to have the potential to treat a variety of autoimmune skin diseases, such as vitiligo and alopecia.

Vitiligo as used herein refers to an autoimmune skin disease characterised by the loss of patches of skin pigmentation. Vitiligo occurs when melanocytes die or stop functioning. This results in a loss of melanin which is responsible for skin pigmentation. The pale areas of skin are more vulnerable to sunburns, making it important that proper precautions are taken by vitiligo patients. The condition can affect the skin on any part of the body, but more typically presents on the face, neck, hands and in skin creases. Vitiligo may also affect hair, or the inside of the mouth.

There are 2 main types of vitiligo—non-segmental and segmental vitiligo. Non-segmental vitiligo is significantly more common than segmental vitiligo, with about 90% of patients having this form of the disease. Patients with non-segmental vitiligo typically have symptoms on both side of the body, i.e. the white patches tend to present as symmetrical patches. In contrast, segmental vitiligo typically presents as white patches on only one area of the body.

There are no treatments currently available which can stop the process of vitiligo. Thus, the white patches caused by vitiligo are usually permanent Available treatments, such as steroid creams, phototherapy or camouflage creams are therefore focussed on reducing the appearance of the white patches.

Alopecia areata, alopecia or spot baldness as used herein refers to an autoimmune skin disorder wherein the hair follicles are targeted by the immune system. This results in the suppression of hair growth and hair loss. The loss of hair typically occurs in clumps and may grow back, although the hair may fall out again. Alopecia can be further sub-classified as:
Diffuse alopecia areata, where the hair loss is diffusely distributed over the whole scalp;
Alopecia areata monolocularis, where hair loss is only in one spot;
Alopecia areata multilocularis, where hair loss occurs over multiple areas;
Ophiasis alopecia areata, where there is a sudden thinning of hair as opposed to hair loss in patches
Alopecia areata barbae, where the hair loss is limited to the beard;
Alopecia areata totallis, where there is total hair loss on the scalp; and
Alopecia areata universalis, where all body hair is lost, including pubic hair.

There are no known cures for alopecia. Treatment options typically include corticosteroids such as clobetasol or fluocinonide, minoxidil, immunosuppressants such as cyclosporine, and diphenylcyclopropenone. Wigs or hairpieces can be used to cover up the bald areas, although some patients may find these uncomfortable to wear.

Vitiligo and alopecia areata are not life-threatening conditions and are not contagious. However, the symptoms of both diseases are highly visible and can therefore also have an adverse effect on the emotional and mental health of patients suffering from either or both diseases.

In this respect, studies have shown that vitiligo and alopecia tend to occur together more commonly in patients compared to pure chance. In addition, both diseases feature a patchy distribution and are minimally symptomatic, compared to other more inflammatory skin conditions like atopic dermatitis or psoriasis. The similarities between the two autoimmune skin diseases further extend beyond shared clinical symptoms—they also share similarities in pathogenesis. For example, increase reactive oxygen species (ROS) and high cellular stress levels are thought to act as the initiating trigger for the innate immune system in both conditions. In addition, studies in mouse models have implicated an IFN-γ-driven immune response and cytotoxic CD8+ T cells as the main pathogenic factors in both diseases. See review by Rork et al, Curr Opin Pediatr 2016 August; 28(4): 463-469. In view of the implication of the IFN-γ signalling pathway in both diseases, ruxolitinib (an inhibitor that targets JAK, a downstream effector of IFN-γ signalling) is currently undergoing clinical trials for both vitiligo and alopecia.

Hence, whilst not wishing to be bound by theory, the present inventors believe that farudodstat has the potential to treat both vitiligo and alopecia by virtue of their various similarities, in particular the crucial role of cytotoxic CD8+ T cells in both diseases.

In one embodiment, the autoimmune disease is selected from the group comprising or consisting of Lichen planus, Hidradenitis suppurativa, Coeliac disease, Ulcerative colitis, Crohn's disease, Graves' disease, Autoimmune thyroiditis, Endometriosis, Multiple sclerosis and Optic neuritis.

Multiple sclerosis (MS) is a disease in which the myelin sheath insulating the nerve cells in the brain and spinal cord are damaged. As a result of the damage, the ability of the nervous system to properly transmit signals is disrupted. This in turn causes a range of serious physical and mental issues, for example double vision, blindness in one eye, muscle weakness, problems with sensation or co-ordination, problems with speech, acute or chronic pain, bladder and bowel difficulties, depression, and mood swings.

The condition begins in most cases as a clinically isolated syndrome over a number of days with the majority suffering from motor or sensory problems. The course of symptoms occurs in two patterns initially, either as episodes of sudden worsening that last a few days to months, known as relapses, followed by improvement in most cases, or a gradual worsening over time without periods of recovery. Relapses are generally unpredictable and occur without warning.

The cause of multiple sclerosis is currently unclear, although the underlying mechanism is thought to be destruction of the myeline sheath by the individual's own immune system, i.e. multiple sclerosis is considered at least in part an autoimmune disease. It is the most common immune-mediated disorder affecting the central nervous system, with about 2.3 million people affected globally. However, there are a number of autoimmune diseases that have a serious and negative impact on the life of many suffers.

In one embodiment, the autoimmune disease is multiple sclerosis (MS). Multiple sclerosis is generally further classified as one of four variants: clinically isolated syndrome (CIS), relapsing-remitting MS (RRMS), primary progressive MS (PPMS) and secondary progressive MS (SPMS). Hence, in one embodiment the autoimmune disease is selected from the group comprising CIS, RRMS, PPMS and SPMS.

Relapsing-remitting MS (RRMS) is characterized by unpredictable relapses followed by periods of months to years of relative quiet (remission) with no new signs of disease activity. The condition is typified by progressive, sustained demyelination, and associated axonal loss. In one embodiment, the autoimmune disease is RRMS.

The attacks—also called relapses or exacerbations (also referred to herein as flare)—are followed by periods of partial or complete recovery (remissions). During remissions, all symptoms may disappear, or some symptoms may continue and become permanent. However, there is no apparent progression of the disease during the periods of remission. RRMS can be further characterized as either active (with relapses and/or evidence of new MRI activity over a specified period of time) or not active, as well as worsening (a confirmed increase in disability following a relapse) or not worsening.

The relapsing-remitting subtype usually begins with a clinically isolated syndrome (CIS). In CIS, a person has an attack suggestive of demyelination, but does not fulfil the criteria for multiple sclerosis. 30 to 70% of persons who experience CIS, later develop MS. In one embodiment, the autoimmune disease is CIS.

Primary progressive MS (PPMS) occurs in approximately 10-20% of individuals, with no remission after the initial symptoms. It is characterized by progression of disability from onset, with no, or only occasional and minor, remissions and improvements. In one embodiment, the autoimmune disease is PPMS.

Secondary progressive MS (SPMS) occurs in around 65% of those with initial relapsing-remitting MS, who eventually have progressive neurologic decline between acute attacks without any definite periods of remission. Occasional relapses and minor remissions may appear. SPMS can be further characterized as either active (with relapses and/or evidence of new MRI activity during a specified period of time) or not active, as well as with progression (evidence of disability accumulation over time, with or without relapses or new MRI activity) or without progression. In one embodiment, the autoimmune disease is SPMS.

In one embodiment the disease is chronic inflammatory demyelinating polyneuropathy. In one embodiment the disease is transverse myelitis. In one embodiment the disease is neuromyelitis optica.

In one embodiment, the autoimmune disease is one or more of the following: rheumatoid arthritis, psoriatic arthritis, ankylosing spondylitis, multiple sclerosis, Wegener's granulomatosis, systemic lupus erythematosus, psoriasis, sarcoidosis, polyarticular juvenile idiopathic arthritis, inflammatory bowel disease such as ulcerative colitis and Crohn's disease, Reiter's syndrome, fibromyalgia and type-1 diabetes. In one embodiment it is not any one of the same.

In one embodiment, the autoimmune disease is psoriatic arthritis. In one embodiment it is not psoriatic arthritis. In one embodiment the autoimmune disease is ankylosing spondylitis. In one embodiment it is not ankylosing spondylitis. In one embodiment, the autoimmune disease is multiple sclerosis. In one embodiment it is not multiple sclerosis. In one embodiment, the autoimmune disease is Wegener's granulomatosis. In one embodiment, it is not Wegener's granulomatosis. In one embodiment, the autoimmune disease is systemic lupus erythematosus. In one embodiment it is not systemic lupus erythematosus. In one embodiment, the autoimmune disease is psoriasis. In one embodiment it is not psoriasis. In one embodiment, the autoimmune disease is sarcoidosis. In one embodiment, it is not sarcoidosis. In one embodiment, the autoimmune disease is polyarticular juvenile idiopathic arthritis. In one embodiment it is not polyarticular juvenile idiopathic arthritis. In one embodiment, the autoimmune disease is an inflammatory bowel disease, such as ulcerative colitis or Crohn's disease. In one embodiment it is not an inflammatory bowel disease, such as ulcerative colitis or Crohn's disease. In one embodiment the autoimmune disease is Reiter's syndrome. In one embodiment it is not Reiter's syndrome. In one embodiment, the autoimmune disease is fibromyalgia. In one embodiment it is not fibromyalgia. In one embodiment, the autoimmune disease is type-1 diabetes. In one embodiment it is not type-1 diabetes. In one embodiment the autoimmune disease is arthritis, such as rheumatoid arthritis. In one embodiment it is not arthritis, such as rheumatoid arthritis.

In one embodiment the autoimmune skin disease is not atopic dermatitis. In one embodiment the autoimmune skin disease is not erythema. In one embodiment the autoimmune skin disease is not scleroderma. In one embodiment the autoimmune skin disease is not lupus. In one embodiment the autoimmune skin disease is not Behcet's disease. In one embodiment the autoimmune skin disease is not bullous pemphigoid. In one embodiment the autoimmune skin disease is not IgA pemphigus.

In one embodiment the autoimmune skin disease is not pemphigoid. In one embodiment the autoimmune skin disease is not ocular cicatricial pemphigoid. In one embodiment the autoimmune skin disease is not pemphigoid gestationis (PG), pemphigus vulgaris (PV), or pemphigus folliaceus (PF). In one embodiment the autoimmune skin disease is not vesiculobullous dermatosis. In one embodiment the autoimmune skin disease is not dermatosmyositis. In one embodiment the autoimmune skin disease is not lichen planus. In one embodiment the autoimmune skin disease is not dermatitis herpetiformis. In one embodiment the autoimmune skin disease is not vesiculobullous dermatosis. In one embodiment the autoimmune skin disease is not linear IgA dermatosis. In one embodiment the autoimmune skin disease is not vasculitis.

### Viral Infections

As used herein, virus refers to a virus that is responsible for a viral infection or viral disease. Thus, a virus in the context of the present disclosure generally refers to a pathogenic virus. It is not intended to refer to a viral vector or virus employed as a therapeutic agent.

Viral infection or viral disease as used interchangeably herein refers to an infection or disease caused by the presence of a virus in the body.

In one embodiment, the virus responsible for the viral infection is an RNA virus.

RNA viruses are generally categorised as Riboviria and Orthornavirae. There are single stranded forms and also double stranded forms (group III in the Baltimore classification) of RNA viruses. Single stranded viruses are divided into: positive sense single stranded RNA viruses (group IV Baltimore classification); negative sense single stranded RNA viruses (group V Baltimore classification) and single stranded RNA viruses with a DNA intermediate (group VI Baltimore classification). Hence, in one embodiment, the virus is a single stranded RNA virus, for example a positive sense single stranded RNA virus or a negative sense single stranded RNA virus.

In one embodiment, the virus responsible for the viral infection is Sarbecovirus. Sarbecovirus is the subgenus of viruses that cause severe acute respiratory syndrome. Examples of Sarbecovirus include SARS-CoV, SARS-CoV2, Bat SARS-like coronavirus WIV1 and Bat coronavirus RaTG13. The genus is betacoronavirus and the family is coronaviridae. Accordingly, in one embodiment the virus is in the Coronaviridae family.

Merbecovirus is the subgenus of viruses that cause Middle East respiratory syndrome-related coronavirus. The genus is betacoronavirus and the family is coronaviridae. Thus, in one embodiment, the virus is Merbecovirus.

The present disclosure also extends to treatment of Embecovirus, (known as group 2a coronavirus) such as Human Coronavirus HKU1. Hence, in one embodiment the virus is a Embecovirus. The present disclosure also includes Nobecovirus (known as group 2d coronavirus).

In one embodiment, the virus responsible for the viral infection is SARS-CoV. SARS-CoV is also referred to a SARS-CoV1 or SARS. In one embodiment the virus is SARS-CoV2. SARS-CoV2 is also referred to a coronavirus or COVID-19.

In one embodiment the virus is MERS-CoV. MERS-CoV is also referred to a Middle East respiratory syndrome coronavirus.

The viral infection may be a mosquito borne viral disease or due to a mosquito borne virus. Common examples of mosquito-borne diseases include malaria, Chikungunya, Dengue, Zika, West Nile virus, and yellow fever. Yellow, Dengue, and chikungunya are transmitted mostly by Aedes aegypti mosquitoes. Other mosquito-borne diseases like epidemic polyarthritis, Rift Valley fever, Ross River fever, St Louis encephalitis, West Nile fever, Japanese encephalitis, La Crosse encephalitis are typically carried by several different mosquitoes.

Thus, in one embodiment, the viral infection is a mosquito-borne viral disease. In other words, in one embodiment, the virus is transmitted by mosquitoes.

In one embodiment the virus is a Flavivirus. Flavivirus is a genus of viruses that include West Nile virus, Dengue virus, tick-borne encephalitis virus, yellow fever virus, Zika virus etc. Flaviviridae is the family. Accordingly, in one embodiment the virus is in the Flaviviridae family.

The present disclosure extends to the treatment of Dengue virus. There are 5 serotypes of the virus, all of which are able to cause the full spectrum of disease. In one embodiment the Dengue virus is any one of serotypes 1 to 5, in particular serotypes 1, 2, 3 or 4. Thus, in one embodiment, the virus is Dengue serotype 1, 2, 3 or 4. In one embodiment the virus is Dengue serotype 1. In one embodiment, the virus is Dengue serotype 2. In one embodiment, the virus is Dengue serotype 3. In one embodiment, the virus is Dengue serotype 4. In one embodiment, the virus is Dengue serotype 5.

In one embodiment, the virus is an Alphavirus. Alphavirus is a genus of viruses that is the sole genus in the Togaviridae family. There are 31 alphaviruses which include Chikungunya virus, Bebaru virus, Getah virus, Mayaro virus, O‘nyong’nyong virus, Ross River virus and Semliki Forest virus.

In one embodiment the virus is selected from the group comprising a Sarbecovirus and a mosquito borne viral disease.

In one embodiment, the Sarbecovirus is SARS-CoV or SARS-CoV-2, in particular SARS-CoV-2.

In one embodiment, the mosquito borne viral disease or viral infection is selected from the group comprising Dengue, Chikungunya and Zika. In one embodiment, the Dengue is Dengue serotype 1, 2, 3 or 4. Thus, in one embodiment, the virus is selected from the group comprising SARS-CoV-2, Dengue, Chikungunya and Zika.

Comprising in the context of the present specification is intended to mean “including”.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

The background contains technical information and may be used as basis for amendment. The invention will now be described with reference to the following examples, which are merely illustrative and should not be construed as limiting the scope of the present invention.

The present applications claims priority from SG10202201038X, incorporated by reference. The priority document(s) may be employed as basis for corrections.

### BRIEF DESCRIPTION OF FIGURES

FIG. 1A shows the route of synthesis for producing the farudodstat dominant stable pharmaceutical (DSP) polymorph

FIG. 1B shows a flow diagram of the process used to synthesize the farudodstat polymorph

FIG. 1C shows a table of the in-process control steps used during the synthesis of the farudodstat polymorph

FIG. 2 shows the XRPD pattern of the farudodstat DSP polymorph

FIG. 3 shows the details of peaks for the XRPD pattern of the farudodstat DSO polymorph

FIG. 4A shows the XRPD results of the anti-solvent method from the 1^{st} experiment

FIG. 4B shows the XRPD results of the anti-solvent method from the 2^{nd} experiment

FIG. 5A shows the XRPD results of the slurry from the 1st experiment (25° C.)

FIG. 5B shows the XRPD results of the slurry from the 2^{nd} experiment (25° C.)

FIG. 5C shows the XRPD results of the slurry from the 3^{rd} experiment (25° C.)

FIG. 5D shows the XRPD results of the slurry from the 4^{th} experiment (50° C.)

FIG. 5E shows the XRPD results of the slurry from the 5^{th} experiment (50° C.)

FIG. 5F shows the XRPD results of the slurry from the 6^{th} experiment (50° C.)

FIG. 6A shows the XRPD results of the solvent-thermal heating/cooling 1^{st} experiment

FIG. 6B shows the XRPD results of the solvent-thermal heating/cooling 2^{nd} experiment

FIG. 7 shows the XRPD results of the evaporation experiments

FIG. 8 shows the XRPD results of the grinding experiments

FIG. 9 shows the XRPD results of the re-crystallisation experiments

FIG. 10 shows the XRPD results of the reactive crystallisation experiments

FIG. 11 shows the XRPD results of the crystalline form of farudodstat from evaporation of THF solution

FIG. 12 shows the XRPD results of the crystalline form of farudodstat from anti-solvent in THF-water

FIG. 13 shows the XRPD result of the spray dried farudodstat sample

FIG. 14 shows the XRPD result of farudodstat from the solvent evaporation experiment

FIG. 15 shows the XRPD result of the freeze-dried farudodstat sample

FIG. 16 shows the XRPD result of the milled farudodstat sample

FIG. 17 shows the TGA and DSG profiles of the farudodstat DSP polymorph

FIG. 18 shows the TGA and DSC profile of the crystalline form of farudodstat from evaporation of THF solution

FIG. 19 shows the TGA and DSC profile of the crystalline form of farudodstat from anti-solvent in THF-water

FIG. 20A shows a polarized light microscopy (PLM) image of farudodstat after milling for 27 hours at 200× magnification

FIG. 20B shows a PLM image of farudodstat after milling for 27 hours at 500× magnification

FIG. 21A shows a PLM image from the re-crystallisation experiment at 200× magnification

FIG. 21B shows a PLM image from the re-crystallisation experiment at 500× magnification

FIG. 22A shows a PLM image from the reactive re-crystallisation experiment using HCl—NaOH at 200× magnification

FIG. 22B shows a PLM image from the reactive re-crystallisation experiment using HCl—NaOH at 500× magnification

FIG. 22C shows a PLM image from the reactive re-crystallisation experiment using NaOH—HCl at 200× magnification

FIG. 22D shows a PLM image from the reactive re-crystallisation experiment using NaOH—HCl at 500× magnification

FIG. 23A shows a PLM image of the crystalline form of farudodstat from evaporation of THE solution at 200× magnification

FIG. 23B shows a PLM image of the crystalline form of farudodstat from evaporation of THE solution at 500× magnification

FIG. 24A shows a PLM image of the crystalline form of farudodstat from anti-solvent in THF-water at 200× magnification

FIG. 24B shows a PLM image of the crystalline form of farudodstat from anti-solvent in THF-water at 500× magnification

FIG. 25A shows XRPD results from the solvent solubility assessment experiment

FIG. 25B shows a summary table of the results of the solubility assessment experiment. *=little solid available for performing XRPD. Pattern 1=XRPD pattern for farudodstat DSP polymorph

FIG. 26 shows XRPD results from bead-milling of farudodstat DSP polymorph

FIG. 27A shows XRPD results from solvent drop grinding experiment

FIG. 27B shows summary table of the results from the solvent drop grinding experiment *WP=white powder, Pattern 1=XRPD pattern for farudodstat DSP polymorph

FIG. 28A shows XRPD results from the thermal cycling experiment

FIG. 28B shows summary table of the results from the thermal cycling experiment WS=wet slurry, CS=clear solution, Pattern 1=XRPD pattern for farudodstat DSP polymorph

FIG. 29A shows XRPD results from room temperature evaporation experiment

FIG. 29B shows summary table of the results from room temperature evaporation experiment Pattern 1=XRPD pattern for farudodstat DSP polymorph.

FIG. 30A shows XRPD results from fast evaporation experiment at 50° C. for 1,3-dioxolane

FIG. 30B shows XRPD results from fast evaporation experiment at 50° C. for 1,4-dioxane

FIG. 30C shows XRPD results from fast evaporation experiment at 50° C. for 2-propanol and 1,1-dimethoxymethane

FIG. 30D shows XRPD results from fast evaporation experiment at 50° C. for THP, THF, 2-methyl THF and 1,1,1.3.3.3-Hexafluoro-2-Propanol.

FIG. 30E shows XRPD results from fast evaporation experiment at 50° C. for 2-ethoxyethanol

FIG. 30F shows XRPD results from fast evaporation experiment at 50° C. for DMF

FIG. 30G show summary table of the results from fast evaporation experiment at 50° C. WS=wet slurry, **=little solid, Pattern 1=XRPD pattern for farudodstat DSP polymorph

FIG. 31A shows XRPD results from fast evaporation experiment at 100° C. for DMA

FIG. 31B shows XRPD results from fast evaporation experiment at 100° C. for DMF

FIG. 32A shows XRPD results from fast cooling experiment of 1,3-dioxolane

FIG. 32B shows summary table of results from fast cooling experiment Pattern 1=XRPD pattern for farudodstat DSP polymorph, WS=wet slurry, CS=clear solution

FIG. 33A shows XRPD results from 2-week slurry experiment

FIG. 33B shows XRPD results from 2-week slurry experiment after fast evaporation at 100° C.

FIG. 33C shows summary table of the results from the 2-week slurry experiment Pattern 1=XRPD pattern for farudodstat DSP polymorph, Pattern 1+=Pattern 1 with additional peaks, WS=wet solid, CS=clear solution

FIG. 34A shows XRPD results from anti-solvent addition (water) experiment

FIG. 34B shows summary table of results from anti-solvent addition (water) experiment Pattern 1=XRPD pattern for farudodstat DSP polymorph

FIG. 34C shows XRPD results from anti-solvent addition (acetone) experiment

FIG. 34D shows summary table of results from anti-solvent addition (acetone) experiment Pattern 1+=Pattern 1+additional peaks

FIG. 34E shows summary table of results from anti-solvent addition (heptane) experiment

FIG. 34F shows summary table of results from anti-solvent addition (MEK) experiment

FIG. 35 shows XRPD results from anti-solvent addition+fast cool experiment for 1,4-dioxane with MEK as anti-solvent

FIG. 36A shows XRPD results from anti-solvent addition experiment at 50° C.

FIG. 36B shows summary table of XRPD results from anti-solvent addition experiment at 50° C. Pattern 1=XRPD pattern for farudodstat DSP polymorph

FIG. 37 shows XRPD diffractogram of farudodstat DSP polymorph (CS-276-22 Pattern 1) in comparison with XRPD diffractogram of 1,1,1.3.3.3-Hexafluoro-2-Propanol polymorph before (Pattern 2) and post-drying (Post-drying Pattern 1)

FIG. 38 shows XRPD peak list for 1,1,1.3.3.3-Hexafluoro-2-Propanol polymorph (Pattern 2)

FIG. 39A shows thermogravimetry (TG)/differential scanning calorimetry (DSC) thermogram of HIFP polymorph before drying (wet)

FIG. 39B shows TG/DSC thermogram of 1,1,1.3.3.3-Hexafluoro-2-Propanol polymorph after drying

FIG. 40A shows ¹H NMR spectrum of 1,1,1.3.3.3-Hexafluoro-2-Propanol polymorph before drying (wet)

FIG. 40B shows ¹H NMR spectrum of 1,1,1.3.3.3-Hexafluoro-2-Propanol polymorph after drying

FIG. 41A shows Fourier-transform infrared spectroscopy (FT-IR) spectrum of 1,1,1.3.3.3-Hexafluoro-2-Propanol polymorph

FIG. 41B shows Fourier-transform infrared spectroscopy (FT-IR) spectrum of farudodstat DSP polymorph

FIG. 42 shows XRPD diffractogram of farudodstat DSP polymorph (CS-276-22 Pattern 1) in comparison with XRPD diffractogram of 1NMP polymorph before (NMP Pattern 3) and post-drying (NMP dry Pattern 1)

FIG. 43 shows XRPD peak list for NMP polymorph (Pattern 3)

FIG. 44 shows TG/DSC thermogram of NMP polymorph

FIG. 45 shows ¹H NMR spectrum of NMP polymorph

FIG. 46 shows a summary diagram of the farudodstat polymorphs identified

### EXAMPLES

### Example 1—Synthesis of Farudodstat Dominant Stable Pharmaceutical (DSP) Polymorph

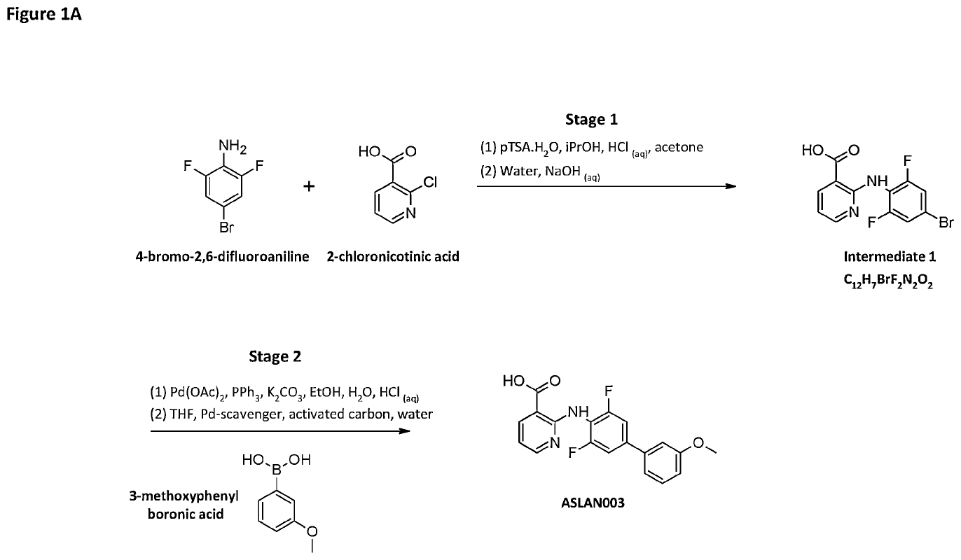

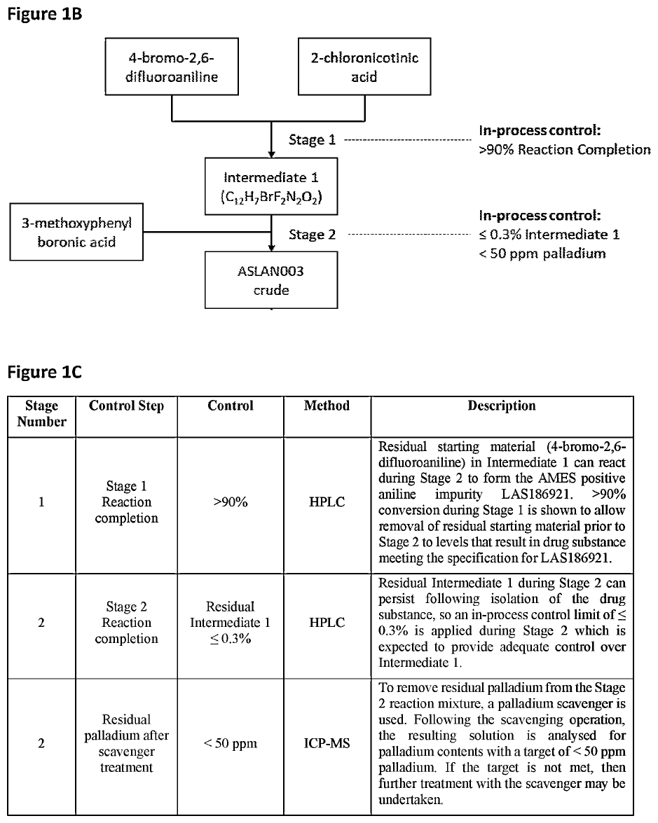

The route of synthesis and flow diagram of the manufacturing process for production of the farudodstat DSP polymorph are shown in FIGS. 1A and 1B respectively. FIG. 1C is a table describing the in-process control steps used during the synthesis of the farudodstat polymorph.

### Stage 1 Process Description

A mixture of 2-chloronicotinic acid, 4-bromo-2,6-difluoroaniline and p-toluenesulphonic acid monohydrate in 2-propanol were heated until reaction completion (>90% conversion). The mixture was cooled and filtered, and the filter cake washed. The product was dried under vacuum to yield crude Intermediate 1, which was taken up in water and the pH adjusted using sodium hydroxide. The resulting slurry was filtered and the filter cake washed with HCl (aq) then acetone. The product was dried under vacuum to yield Intermediate 1 (C₁₂H₇BrF₂N₂O₂) in 70-80% yield.

### Stage 2 Process Description

A mixture of the Intermediate 1 (C₁₂H₇BrF₂N₂O₂), 3-methoxyphenylboronic acid, potassium carbonate, triphenylphosphine and palladium acetate in ethanol and water was heated until reaction completion (0.3% Intermediate 1). Hydrochloric acid was charged, and the resulting slurry was filtered, washing the cake with water. The product was dried under vacuum to yield crude farudodstat. The crude product was dissolved in tetrahydrofuran and treated with a metal scavenger, before being filtered with the aid of activated carbon. The resulting solution was tested for residual palladium levels (<50 ppm). If the in-process test for residual palladium does not meet the specification, then the scavenging procedure may be repeated until the specification is met to ensure effective removal of residual palladium.

### Example 2—Materials and Methods

### 1. Reagents

TABLE 1
List of Reagents used

| Name | Grade | Company | Lot No. |
|---|---|---|---|
| Acetonitrile | HPLC | Merck | II2IF62526 |
| Ethanol | HPLC | Sigma | 04096TK |
| Methanol | HPLC | Merck | SB0SF60097 |
| Isopropanol | HPLC | Merck | IC1IF61120 |
| Ethyl acetate | AR | Yixing Secondary | 070501 |
| | | Chemical | |
| Isopropyl acetate | CP | Shanghai Chemical | 20110419 |
| | | Regent | |
| Acetone | AR | Sinopharm Chemical | T20081202 |
| | | Regent | |
| MIBK | AR | Sinopharm Chemical | T20080411 |
| | | Regent | |
| MEK | AR | Sinopharm Chemical | T20070315 |
| | | Regent | |
| Dichloromethane | AR | Enox | 20110315 |
| MTBE | AR | Fluka | 1359496 |
| THE | HPLC | Merck | IL81F58153 |
| 1,4-Dioxane | AR | Enox | 20110415 |
| DMSO | HPLC | Sigma | 27496KK |
| DMF | AR | Enox | 20110601 |
| Toluene | AR | Enox | 20110315 |
| Heptane | AR | Shantou Xiling | 081110 |
| | | Chemical | |
| I-butanol | AR | Shanghai Runjie | 20110305 |
| | | Chemical Regent | |
| 2-MeTHF | HPLC | Sigma | STBD4579 V |
| Cyclohexane | AR | Shanghai Chemical | 20120713 |
| | | Regent | |
| CHCl3 | AR | Shanghai Lingfeng | 20101025 |
| | | Chemical Regent | |
| NMP | HPLC | Sigma | STBD1943V |
| Acetic acid | AR | Sigma | SZBC2350V |

### 2. Instruments

Milli-Q Direct 8 Water Purification Equipment; Sartorius CP 225D Balance; Mettler-Toledo XP6 Balance; Eppendorf Thermomixer Comfort; Bruker D8 Advance X-ray powder diffractometer (XRPD); Nikon LV100 Polarized Light Microscopy (PLM); TA Q2000 Differential Scanning Calorimetry (DSC); TA Q5000IR Thermal Gravimetric Analysis (TGA), Crystal 16, AVANTIUM; Rotavapor R-114; Planetary Momo Mill (pulverisette-6); Freeze dryer (Free zoon-6)

### 3. Instrument Parameters

### X-Ray Powder Diffractometer (XRPD)

Approximately 5-10 mg samples were run on XRPD using the following parameters:

Tube: Cu: K-Alpha (λ=1.54179 Å); Generator: Voltage: 40 kV; Current: −40 mA; Scan Scope: 4 to 40 deg; Sample rotation speed: 15 rpm; Scanning rate: 10 deg./min

### Differential Scanning Calorimetry (DSC) and Modulate DSC

Samples (˜1 mg) were run using crimped aluminium pans heated from 25° C. to 280° C. at a rate of 10° C./min.

### Thermal Gravimetric Analysis (TGA) Methods

Samples (2˜5 mg) were placed in an open platinum pan and heated from room temperature to 300° C. at a rate of 10° C./min.

### Polarized Light Microscope (PLM)

Samples were dispersed in silicon oil and were observed using an objective lens (10×) and physical lens (20× and 50×) under a crossed polarizer. This results in an overall magnification of 200× and 500×.

### Example 3—Characterization of Physicochemical Properties of Farudodstat DSP Polymorph

The farudodstat DSP polymorph was characterized using X-ray powder diffraction (XRPD), differential scanning calorimetry (DSC), thermogravimetric analysis, (TGA) and polarized light microscopy (PLM). Then it was subjected to various recrystallisation and analysis to confirm it was a DSP polymorph.

### Grinding Experiments

30 mg of the DSP polymorph was ground using a mortar & pestle for 15 mins followed by a check by XRPD. During the grinding process, a few drops of THF were added to the sample. If a new form was identified, it would be further characterized by DSC and TGA. The XRPD results are shown in FIG. 8.

### Re-Crystallization Experiments

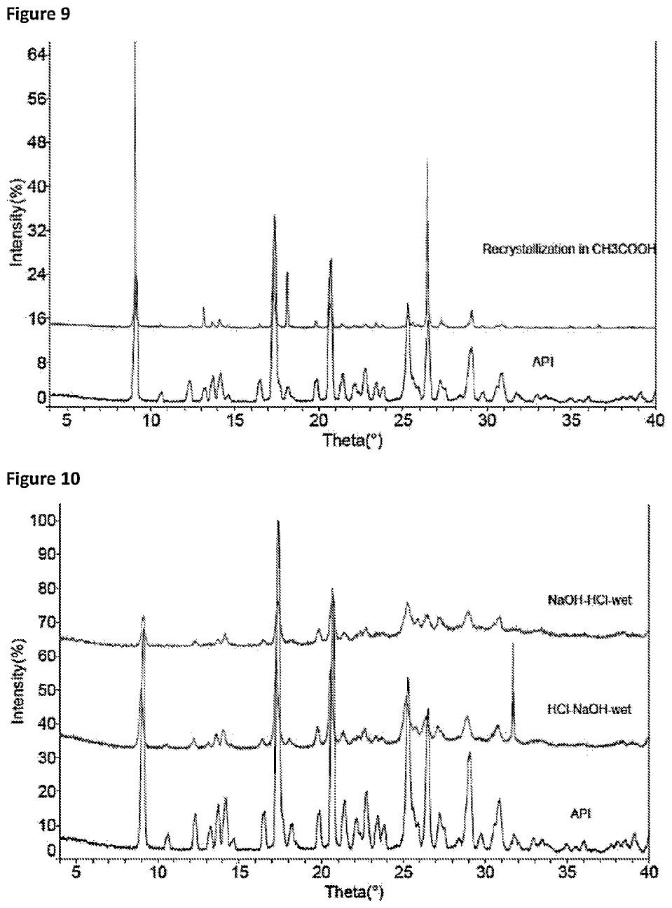

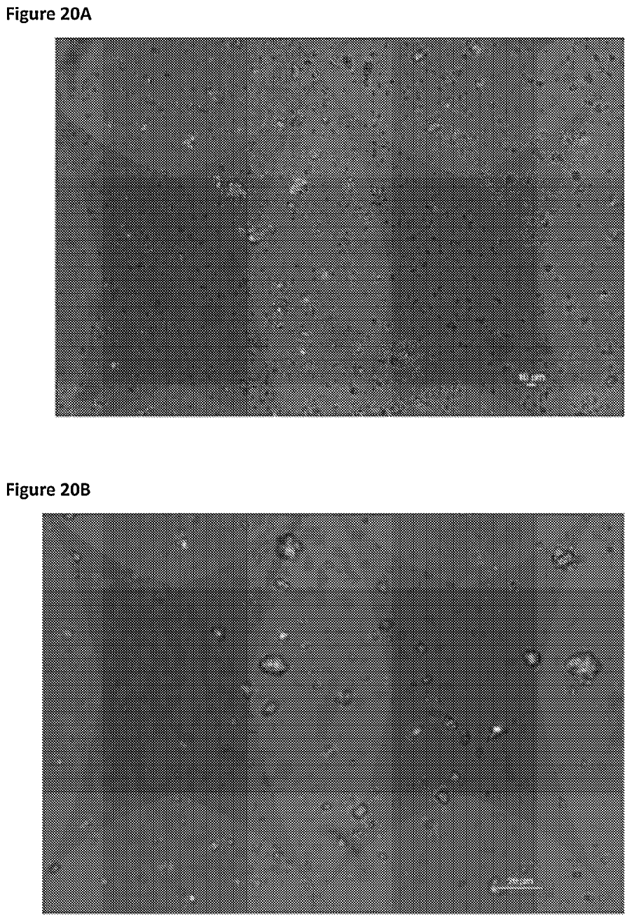

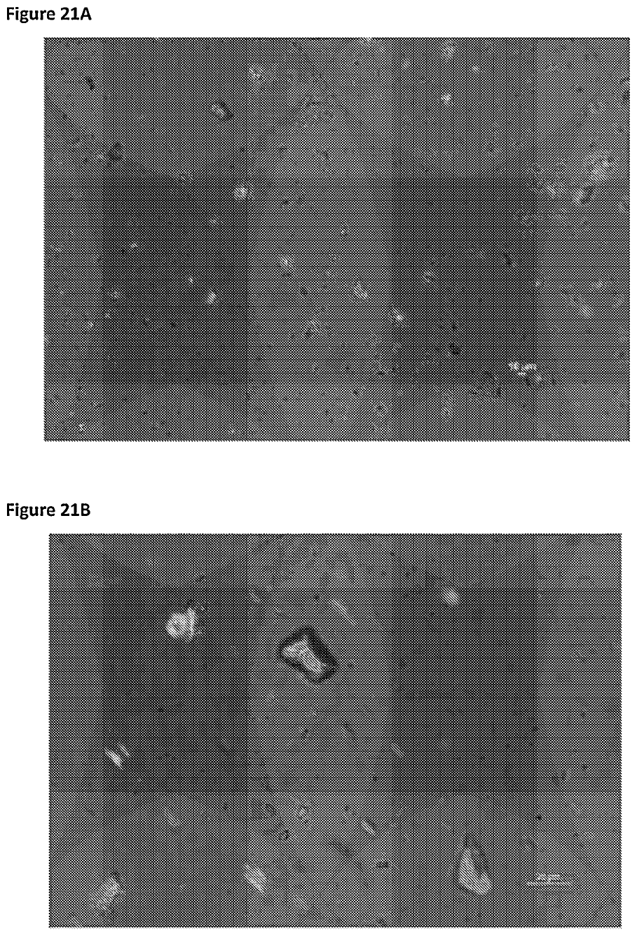

About 3 mg of farudodstat raw material dissolved with acetic acid, resulting in a clear solution at the room temperature. The sample was heated to 55° C., and more materials added in until no more materials could be dissolved. After this, the sample was filtered and stored at room temperature, allowing it to re-crystallize for about 20 hrs, before they were analysed by XRPD and PLM. If a new form was identified, it was further characterized by DSC and TGA. The XRPD results are shown in FIG. 9. The PLM images are shown in FIGS. 20A and 20B.

### Reactive Crystallization Experiments

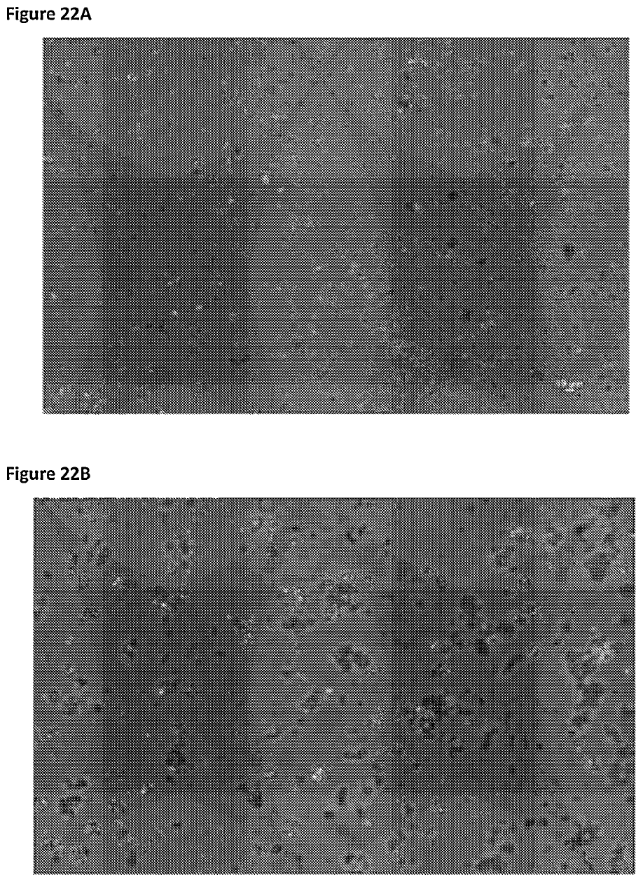

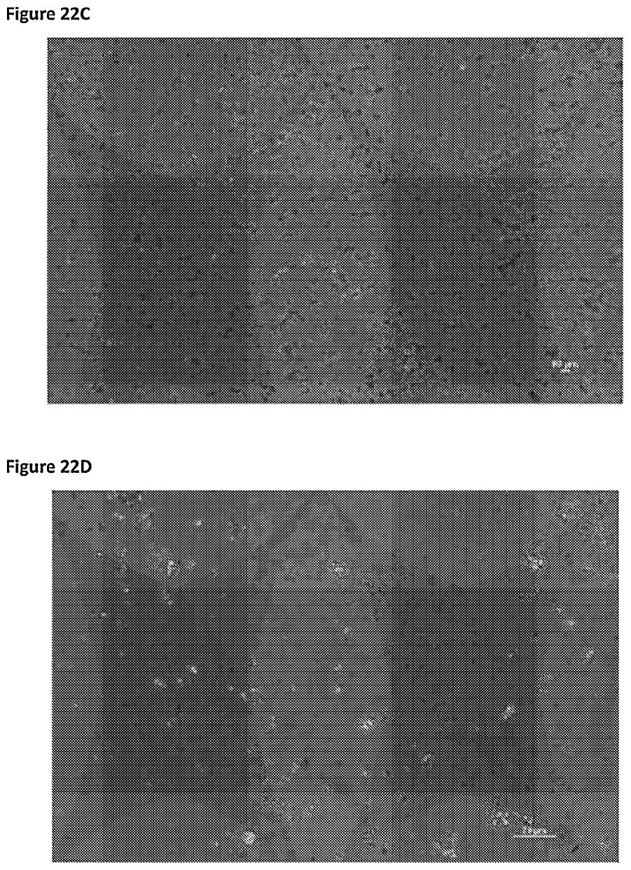

About 100 mg of the DSP polymorph was dissolved with a basic solvent (1N NaOH) to produce a clear solution. After the solution was filtered, an acid solution (1N HCl) was mixed in to precipitate out the drug substance, which was then separated and analysed by XRPD and PLM. If a new form was identified, it was further characterized by DSC and TGA. The XRPD results are shown in FIG. 10. The PLM images are shown in FIGS. 22A to 22D.

### Preparation of Amorphous Sample

### Spray Drying

About 1.2 g of the DSP polymorph was dissolved with THF solvent to produce a clear solution. The solution was then spray dried to produce an amorphous solid dispersion. The parameters used for the spray drying are shown below:

TABLE 2
Parameters for spray drying

| Parameters | Raw material/THF |
|---|---|
| Nozzle orifice size (mm) | 0.6 |
| Inlet air speed (m³/min) | 0.35 |
| Inlet air temperature (° C.) | 75 |
| Chamber out temperature (° C.) | 59 |
| Cyclone in temperature (° C.) | 43 |
| Upper chamber pressure (mBar) | 3.2 |
| Cyclone pressure (mBar) | 23 |
| Set nozzle airflow (L/min) | 10 |
| Set pump speed control | 120(Approx 2.9 mL/min) |
| Cyclone size | medium |

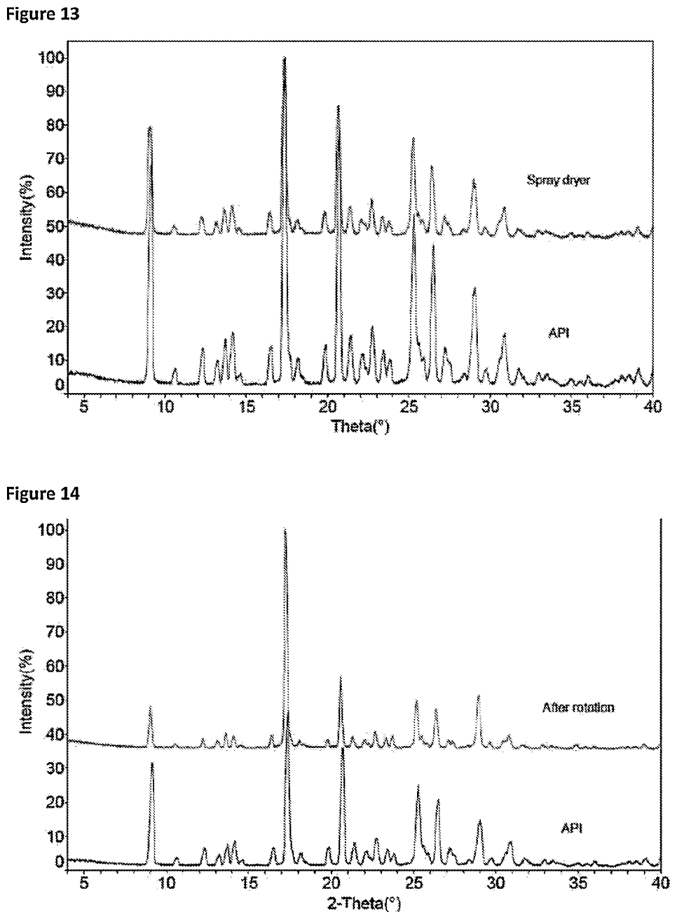

The spray-dried sample was analysed by XRPD and the results are shown in FIG. 13.

### Solvent Evaporation

About 260 mg of farudodstat raw material was dissolved with THF solvent to produce a clear solution. The solution was then concentrated to dryness using a rotary evaporator at a temperature of about 40° C. The residual solid was analysed by XRPD. The XRPD results are shown in FIG. 14.

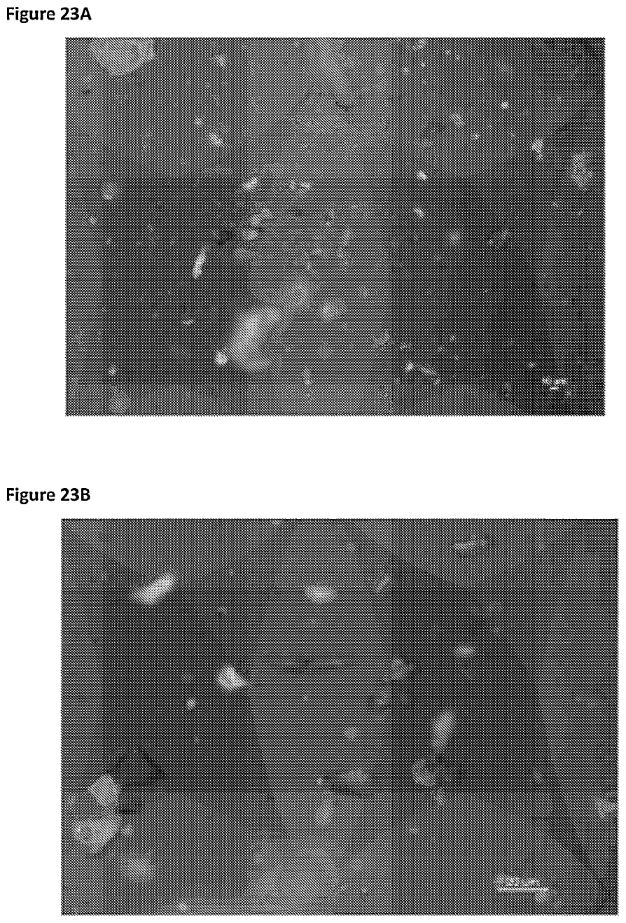

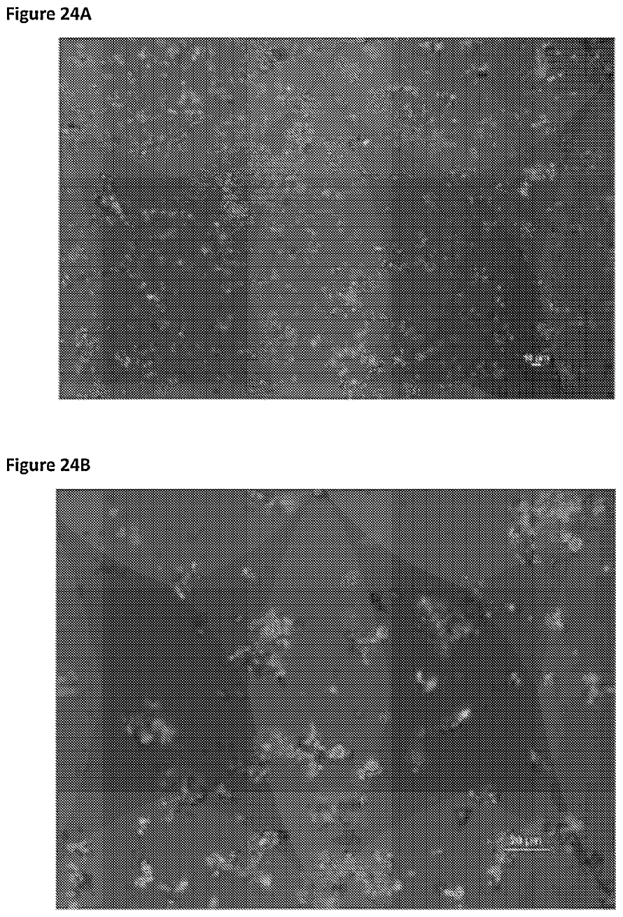

The PLM results are shown in FIGS. 23A and 23B.

### Freeze Drying

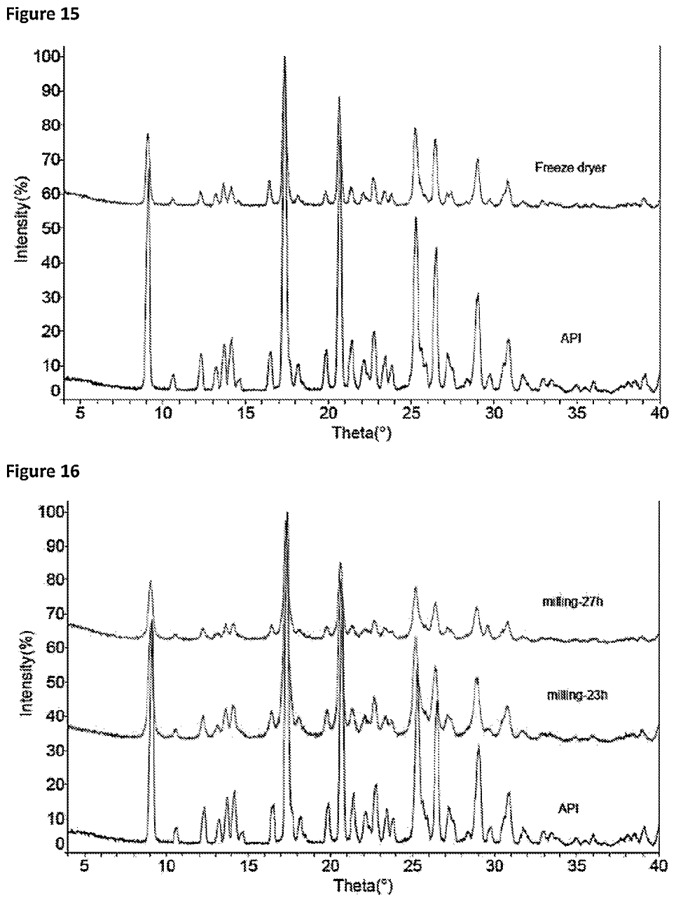

About 30 mg of farudodstat raw material was dissolved with DMSO to produce a clear solution, which was then cooled with dry ice in acetone. Next, water was added to the sample before it was cooled again. Finally, the frozen solution was freeze-dried before the residual solid was analysed by XRPD. The XRPD results are shown in FIG. 15.

### Milling Experiments

About 1.2 g of farudodstat raw material was placed in a ball grinder. The sample was milled for about 24 cycles, comprising milling followed by a 30 min pause. After this, the sample was analysed by XRPD and PLM. The XRPD results are shown in FIG. 16. The PLM results are shown in FIGS. 20A and 20B.

### Characterization of the DSP Polymorph

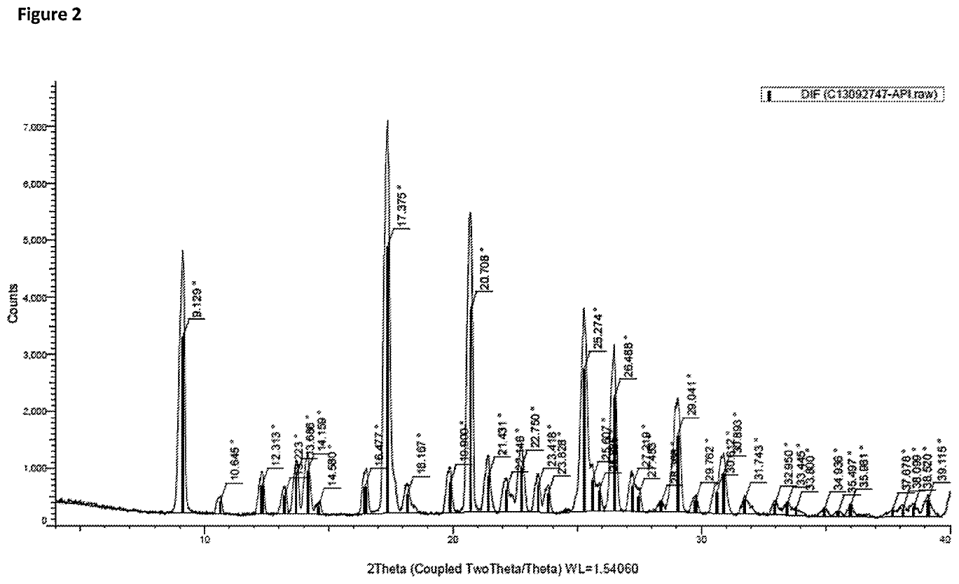

FIG. 2 shows the XRPD of the farudodstat DSP polymorph.

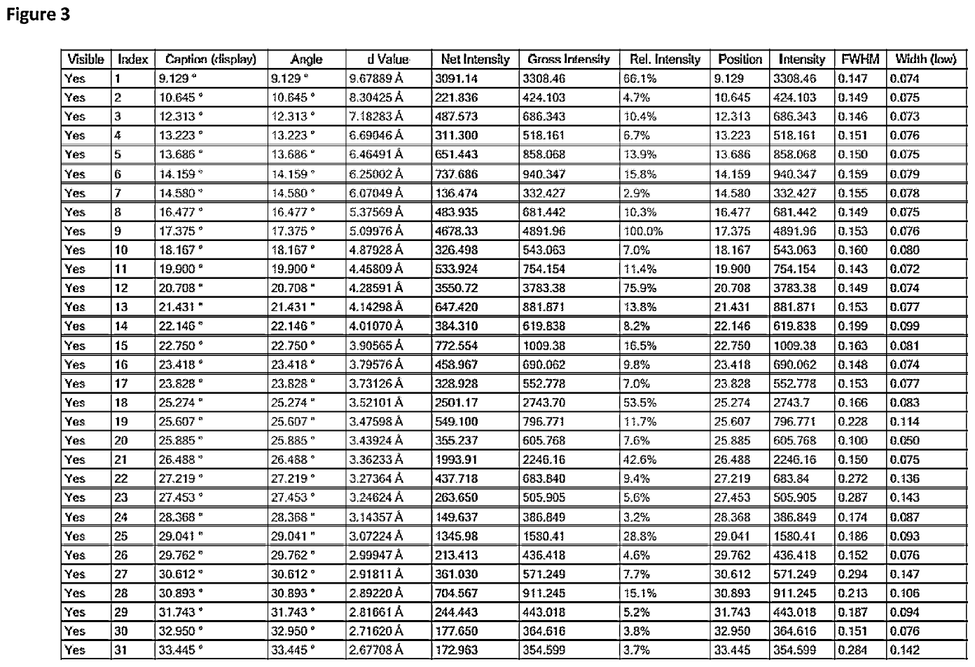

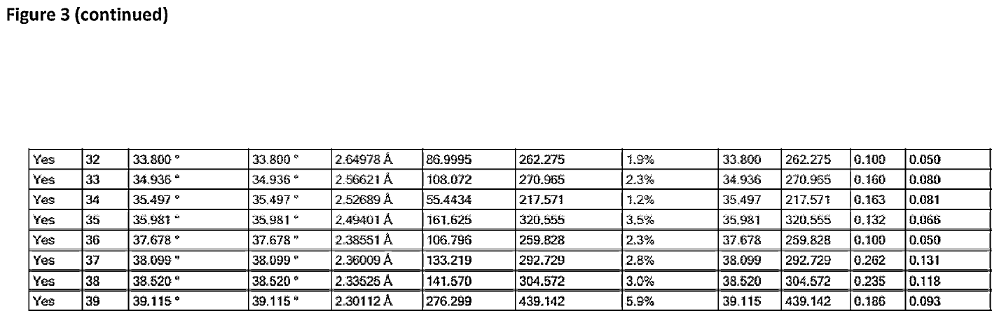

FIG. 3 shows the detailed locations of the peaks in FIG. 2.

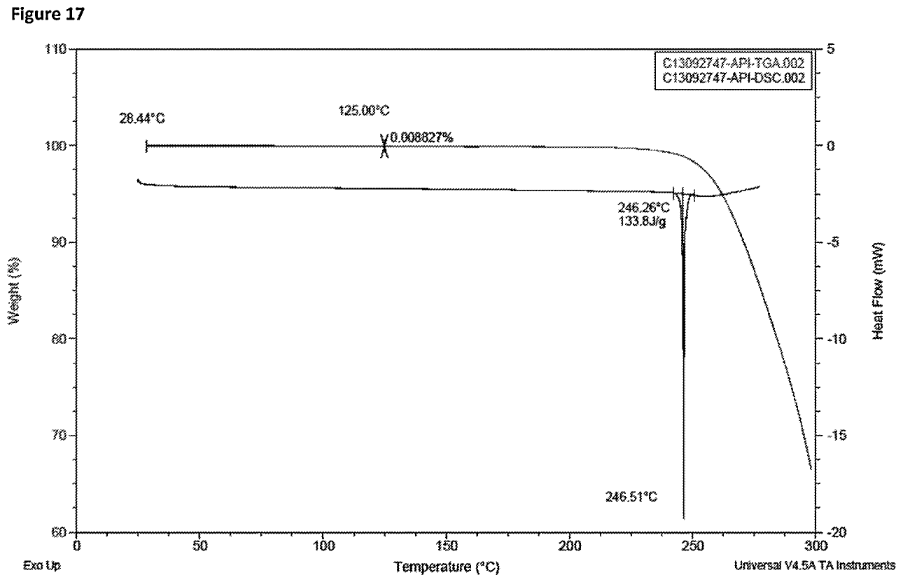

FIG. 17 shows the TGA and DSG profiles of the DSP polymorph.

### Example 4—Approximate Solubility of the DSP in Different Solvents and Solvent Mixtures

The approximate solubility was tested at room temperature by manual dilution combined with visual observation. The solubility of material in different single and mixed solvents was determined at the concentration once no particles of farudodstat could be visually observed in solvents.

The results of the solubility study of the farudodstat DSP polymorph in 43 solvent systems was shown in Table 3 below.

High solubility (more than 100 mg/ml) was observed for some solvents. In particular, a relatively high solubility of DSP polymorph at room temperature was observed when it was dissolved in THF, 2-MeTHF, THF-MeOH (3:1), THF-CAN (3:1), THF-dichloromethane (3:1), THF-Heptane (3:1), THF-water (3:1) and THF-MeOH (1:1).

Conversely, there were 11 organic solvents/mixed solvents that had relatively poor solubility (less than 10 mg/ml).

TABLE 3
Approximate solubility of farudodstat
DSP polymorph in different solvents

| Solvents/Mixed | Solubility | Solvents/Mixed | Solubility |
|---|---|---|---|
| solvents | (mg/mL) | solvents | (mg/mL) |
| MeOH | −1.54 | 1,4-Dioxane | −12.28 |
| EtOH | −0.69 | Water | <0.53 |
| IPA | −0.66 | Dichloromethane | <0.60 |
| 1-butanol | −0.76 | THF-Dichloromethane | −8.34 |
| | | (1:1) | |
| ACN | −0.80 | THF-Dichloromethane | −15.16 |
| | | (3:1) | |
| Acetone | −4.35 | Acetone:Dichloromethane | <1.58 |
| | | (1:1) | |
| MEK | −5.12 | Acetone:Dichloromethane | <1.90 |
| | | (3:1) | |
| MIBK | −0.70 | THF-Heptane (1:1) | <1.51 |
| EtOAc | −1.74 | THF-Heptane (2:1) | −14.35 |
| iPrOAc | −1.27 | THF-Heptane (3:1) | −16.82 |
| MTBE | −1.22 | CHCl₃ | <0.57 |
| THF | −63.88 | THF-Water (1:1) | −1.86 |
| 2-MeTHF | −16.45 | THF-Water (2:1) | −10.92 |
| Toluene | <0.53 | THF-Water (3:1) | −32.90 |
| Heptane | <0.81 | THF-MeOH (1:1) | −20.13 |
| Cyclohexane | <0.49 | THF-MeOH (1:2) | −9.38 |
| THF-MeOH (3:1) | −35.01 | Dioxane-ACN (3:1) | −0.96 |
| THF-ACN (1:1) | −19.59 | DMF | −211.70 |
| THF-ACN (1:2) | −6.25 | DMSO | −170.73 |
| THF-ACN (3:1) | −35.25 | Acetone-Water (5:1) | −3.09 |
| Dioxane-ACN (1:1) | −1.20 | NMP | −206.45 |
| CH₃COOH | −3.28 | | |

### Suspension Equilibration (Slurry) of DSP Polymorph

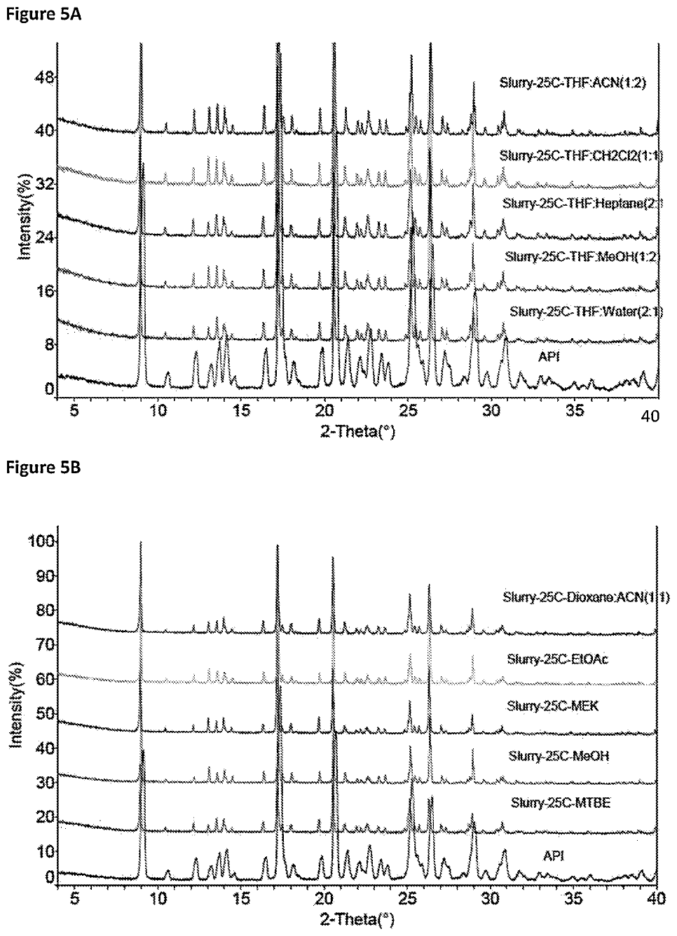

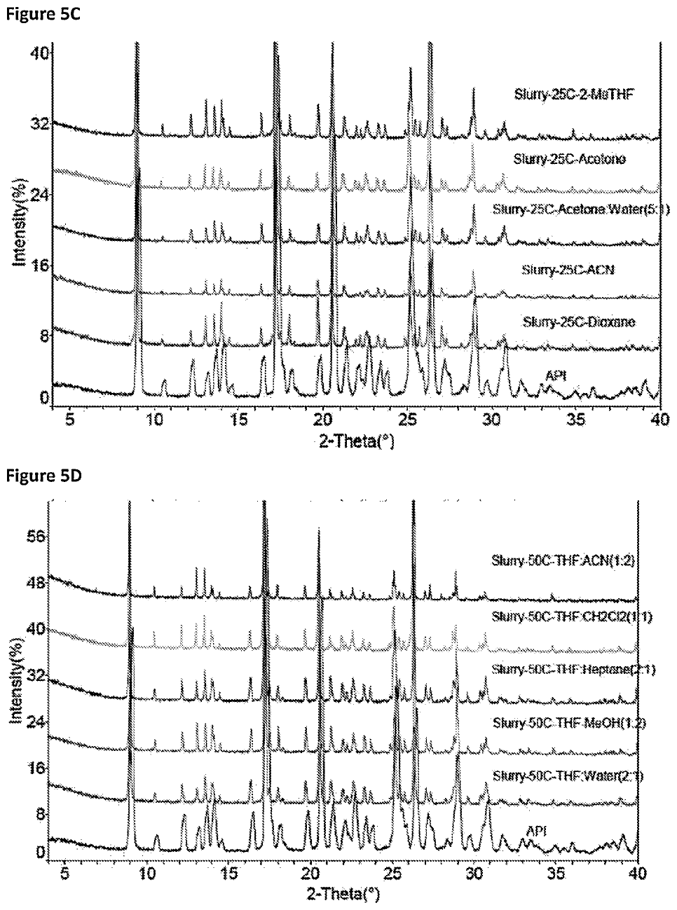

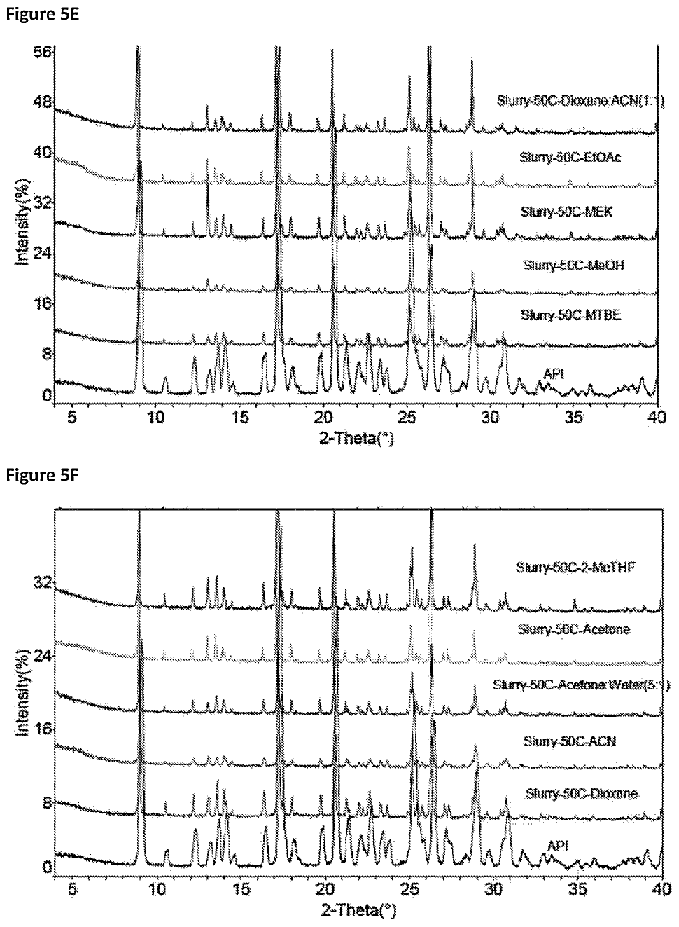

The suspensions of the DSP polymorph were prepared in different single solvents and mixed solvents (see Table 4 below). After this the samples were kept shaking for 72 hrs at 25° C. and 50° C. before they were centrifuged at 14,000 rpm for 10 mins until solid material was separated from the supernatant. The centrifuged samples were investigated by XRPD. The samples were dried under vacuum conditions at 40° overnight and investigated by XRPD, DSC and TGA if new XRPD patterns were found. The XRPD results are shown in FIGS. 5A to 5F.

### Example 5—Testing the Ability to Form the DSP Polymorph after Processing/Treatment

The farudodstat DSP polymorph was subject to processing or treated to test the physical stability of the form.

### Anti-Solvent Precipitation (Anti-Solvent)

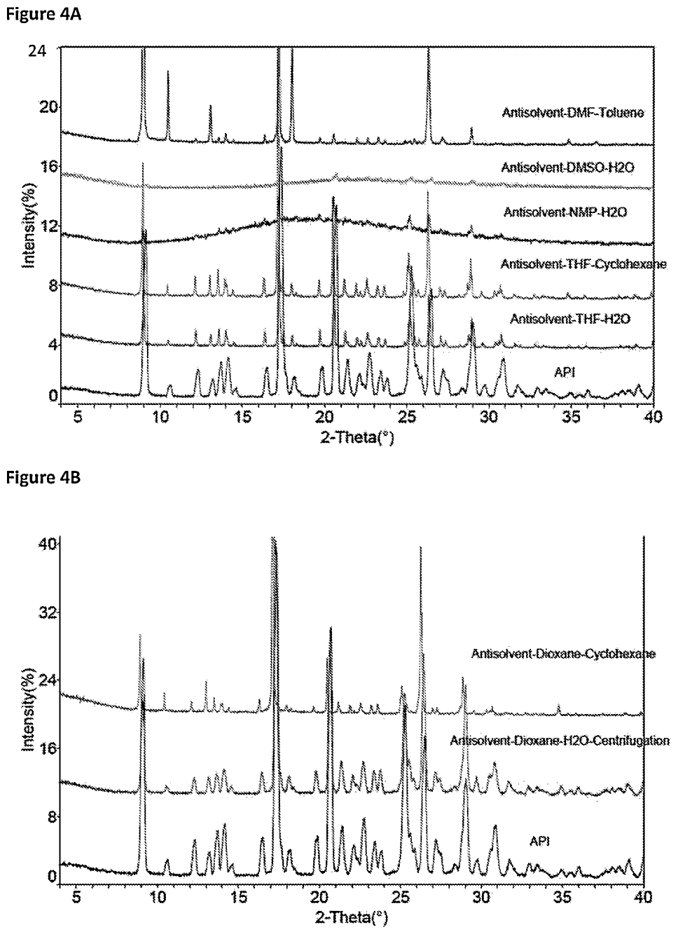

50 mg of the DSP polymorph was dissolved in the selected solvents (see Table 4) according to the solubility results to form saturated solutions. Next, the anti-solvent was slowly added into the solution under stirring until the precipitation formed. If there was no solid precipitation at room temperature, the sample was transferred into the freezer and stored at −20° C. After this, the slurries were stirred for 24 hours before they were centrifuged at 14,000 rpm for 10 mins, until solid material was separated from the supernatant. The centrifuged samples were investigated by XRPD. The samples were next dried under vacuum conditions at 40° C. overnight. If crystalline XRPD patterns were found, the samples were further investigated by XRPD, DSC and TGA. The XRPD results are shown in FIGS. 4A and 3B.

The test parameters are set out in Table 4.

TABLE 4
Screening experiments

| Method | Solvents | Anti-solvents | Temperature |
|---|---|---|---|
| Anti-solvent | THF | H₂O | RT |
| | THF | Cyclohexane | |
| | DMF | H₂O | |
| | DMSO | H₂O | |
| | NMP | H₂O | |
| | 1,4-Dioxane | H₂O | 50° C. |
| | 1,4-Dioxane | Cyclohexane | |
| | DMF | Toluene | RT |
| | DMSO | Toluene | −20° C. |
| | NMP | Toluene | |
| Grinding | THF | N/A | RT |
| Reactive | NaOH | HCl | |
| crystallization | | | |
| Re-crystallization | Acetic acid | N/A | 55° C.~RT |
| Spray dryer | THF | N/A | 75° C. |
| Rotation | THF | N/A | 40° C. |
| Freeze dryer | DMSO | H₂O | −70° C. |
| Milling | N/A | N/A | RT |
| Solvent-thermal | MeOH | N/A | 60° C.~0° C. |
| heating/cooling | Acetone | N/A | 50° C.~0° C. |
| | MEK | N/A | 60° C.~0° C. |
| | Ethyl acetate | N/A | |
| | Dioxane | N/A | |
| | Isopropyl acetate | N/A | |
| | 2-MeTHF | N/A | |
| | THF:H₂O (1:1) | N/A | |
| | THF:CH₂Cl₂ (1:1) | N/A | |
| Slurry | Methanol | N/A | 25° C. |
| | ACN | N/A | |
| | Acetone | N/A | |
| | MEK | N/A | |
| | EtOAc | N/A | |
| | MTBE | N/A | |
| | 2-MeTHF | N/A | |
| | 1,4-Dioxane | N/A | |
| | THF-CH₂Cl₂ (1:1) | N/A | |
| | THF-Heptane (2:1) | N/A | |
| | THF-MeOH (1:2) | N/A | |
| | THF-ACN (1:2) | N/A | |
| | Dioxane-ACN (1:1) | N/A | |
| | Acetone-Water (5:1) | N/A | |
| | THF-Water (2:1) | N/A | |
| | Methanol | N/A | 50° C. |
| | ACN | N/A | |
| | Acetone | N/A | |
| | MEK | N/A | |
| | EtOAc | N/A | |
| | MTBE | N/A | |
| | 2-MeTHF | N/A | |
| | 1,4-Dioxane | N/A | |
| | THF-CH₂Cl₂ (1:1) | N/A | |
| | THF-Heptane (2:1) | N/A | |
| | THF-MeOH (1:2) | N/A | |
| | THF-ACN (1:2) | N/A | |
| | Dioxane-ACN (1:1) | N/A | |
| | Acetone-Water (5:1) | N/A | |
| | THF-Water (2:1) | N/A | |
| Evaporation | THF | N/A | |
| | THF-Water (3:1) | N/A | RT |
| | THF-MeOH (3:1) | N/A | |
| | THF-ACN (3:1) | N/A | |
| | DMF | N/A | |
| | DMSO | N/A | |
| | NMP | N/A | |

All of the experiments confirmed the DSP polymorph was the predominant form. No precipitation was obtained for: anti-solvent DMSO, NMP and evaporation NMP.

Spontaneous Precipitation from Saturated Solutions (Evaporation)

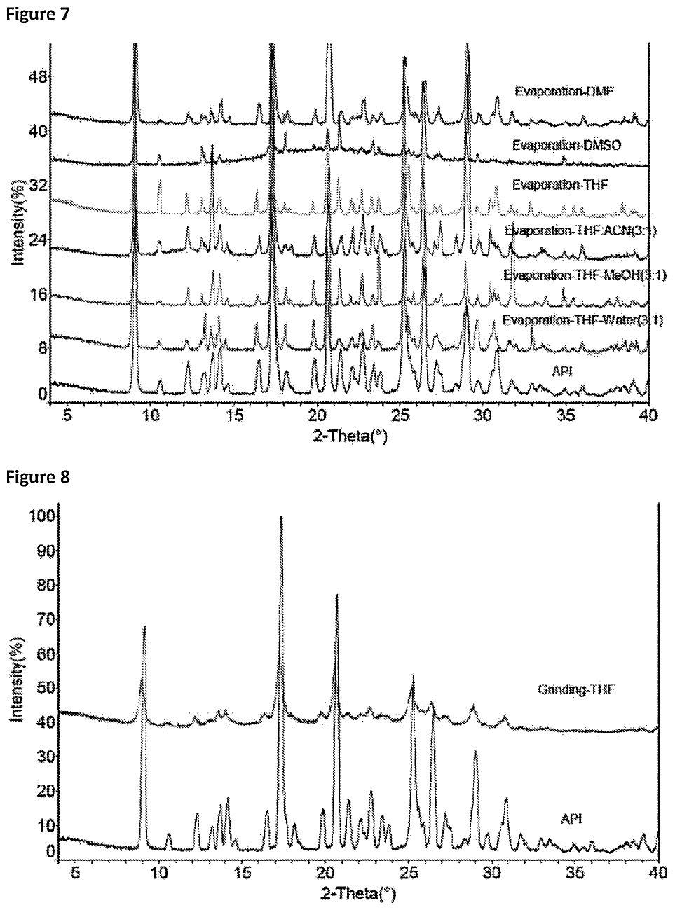

Due to the high solubility of the DSP polymorph in some solvents (see Table 4), these solvents were chosen for the evaporation test. The solutions were prepared according to the solubility of the compound and were kept open at room temperature to let the solvents evaporate to form crystals. After evaporation was complete, the dried solids were collected and investigated by XRPD. Further tests were performed on DSC and TGA if new XRPD patterns were found. The XRPD results are shown in FIG. 7.

### Solvent-Thermal Heating/Cooling Experiments

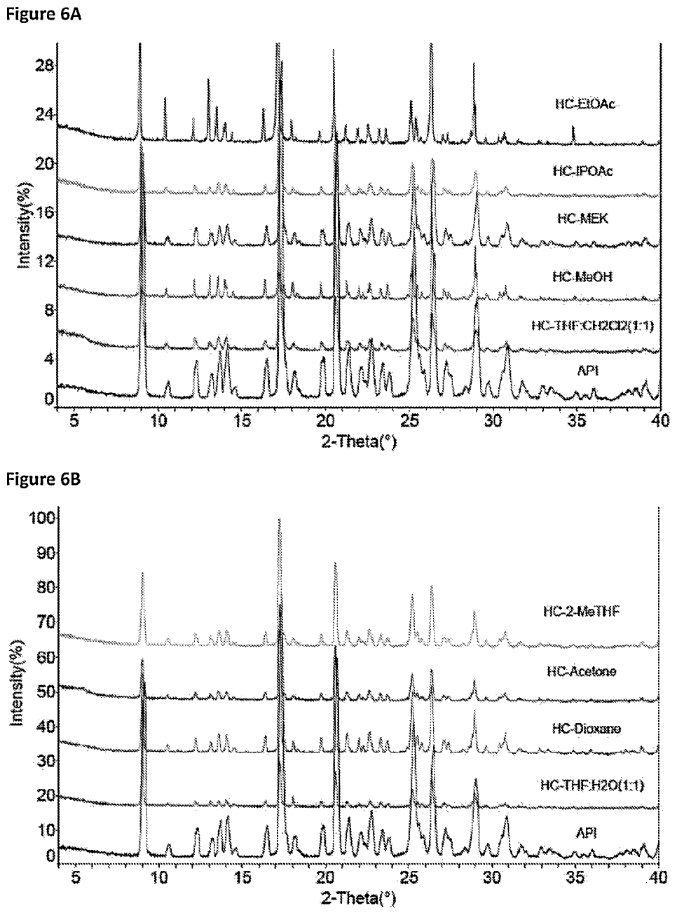

10-30 mg of the original form of ASLAN003 was loaded into glass vials and placed in a thermomixer at either 60° C. or 50° C. After this, the selected solvents (see Table 4) were slowly added to the samples until the compound was completely dissolved. Next, the samples were transferred into the Crystal 16 crystallizer and the cooled to 0° C. at a rate of 0.5° C./min, before they were held at 0° C. for 24 hrs. If there was no solid precipitation at 0° C., the sample was transferred into a freezer at −20° C. Once the cooling process was complete, the samples were centrifuged at 14,000 rpm for 10 mins, until solid material was separated from the supernatant. The wet centrifuged samples were investigated by XRPD. The samples were next dried under vacuum conditions at 40° C. overnight. If crystalline XRPD patterns were found, the samples were further investigated by XRPD, DSC and TGA. The XRPD results are shown in FIGS. 6A and 6B.

### Example 6—Further Characterization of Farudodstat DSP Polymorph

Two samples from Example 4:
1. THF solvent by evaporation; and
2. THF with water by antisolvent
   were further characterized after drying under vacuum conditions at 40° C. overnight and evaluated.

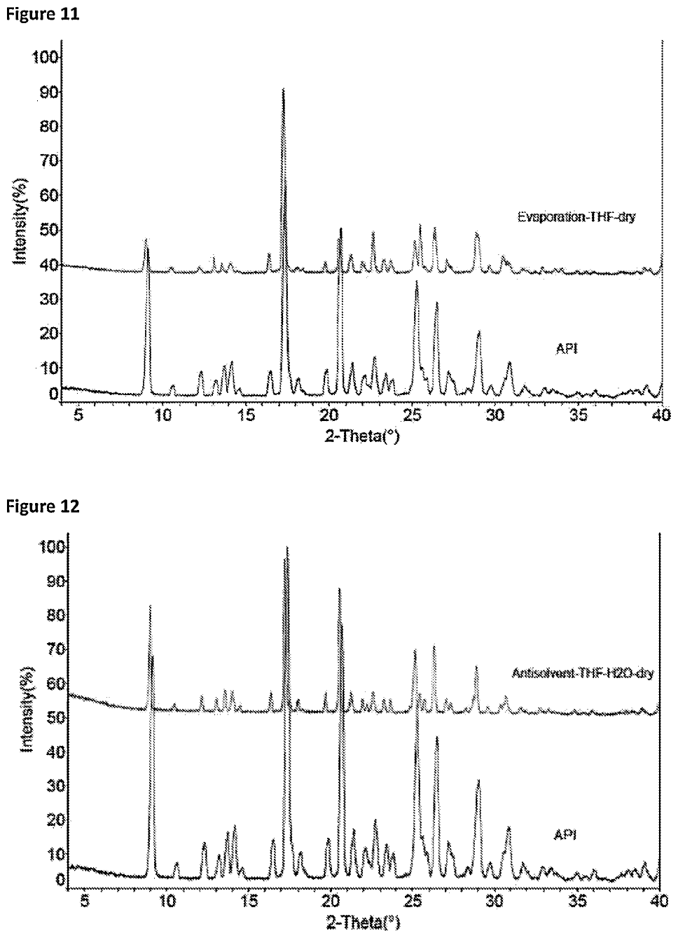

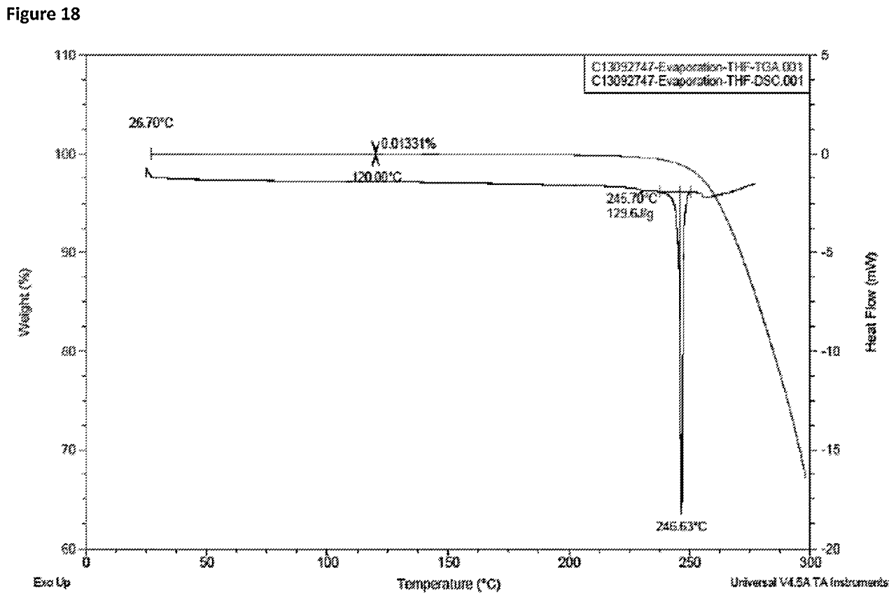

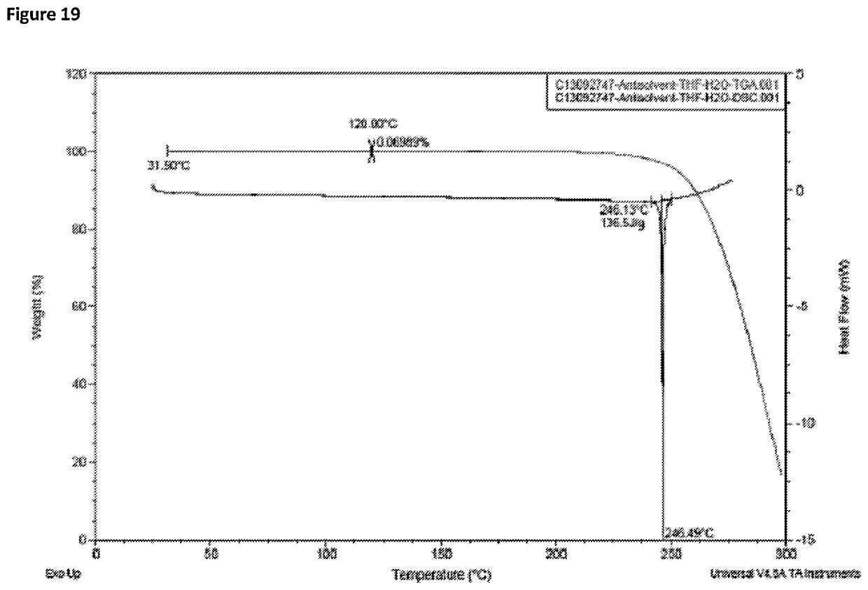

The PLM results are shown in FIGS. 23 and 24. The XRPD results are shown in FIGS. 11 and 12. The TGA and DSC results of the sample in the mixture of method are shown in FIGS. 18 and 19. The results for the sample in THF solvent by evaporation method and the sample in the mixture of THF with water by antisolvent method confirm that the polymorph is stable.

### Conclusions

The experiments indicate that the farudodstat DSP polymorph exhibits good crystallinity and high melting point with minimum weight loss after heating to 200° C.

The farudodstat DSP polymorph was recrystallized from a number of solvents and was stable and reproducible. This suggests that it is the dominant stable pharmaceutical polymorph (DSP).

### Example 7—Farudodstat DSP Polymorph Particle Size Distribution (PSD)

TABLE 5
PSD results

Particle Size (μm)

| Lot No. | MV | D₁₀ | D₅₀ | D₉₀ |
|---|---|---|---|---|
| 1302NC0160A-01 | 6.70 | 2.54 | 5.97 | 11.79 |
| 1412NC0160A-01 | 151.47 | 32.34 | 160.95 | 238.22 |
| 1412NC0160A-02 | 13.27 | 0.90 | 3.46 | 45.05 |

The data shows that the farudodstat polymorph is the dominant stable pharmaceutical polymorph.

### Example 8—Farudodstat Polymorph Screening Experiments

A solvent solubility screen was carried out on the farudodstat DSP polymorph (also referred to as CS-276-22 in the following examples) to determine its solubility in various solvents, and to gain an initial assessment of the polymorphic landscape. To ˜20 mg samples of predominantly amorphous farudodstat, aliquots (2×50 μL and then 100 μL) of the chosen solvents were added until dissolution was observed, or 2 mL had been added. The samples were manually agitated and heated to ˜40° C. in a block attached to a water bath in between each aliquot Clear solutions were uncapped and allowed to evaporate at room temperature. Any solids obtained from slurries and from evaporation were analysed by X-ray powder diffraction (XRPD).

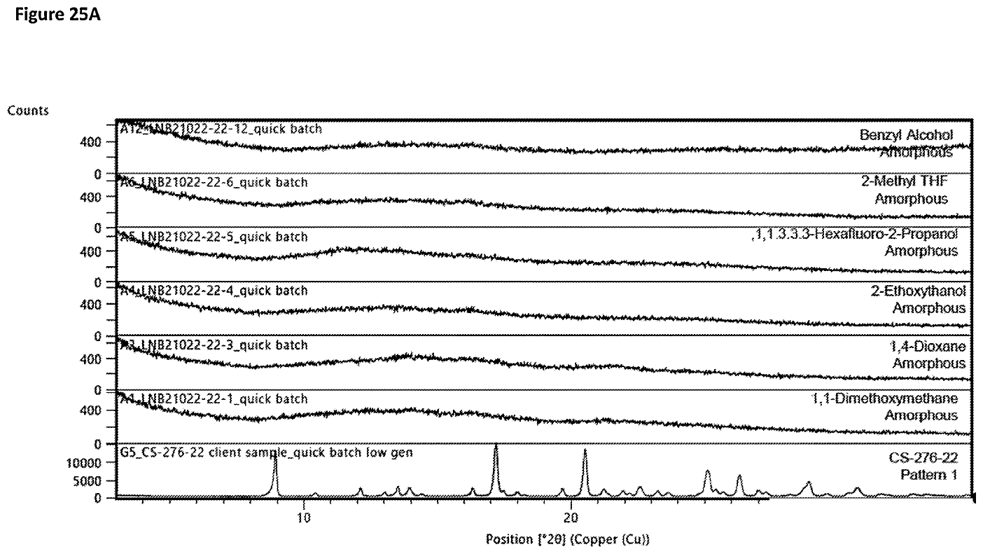

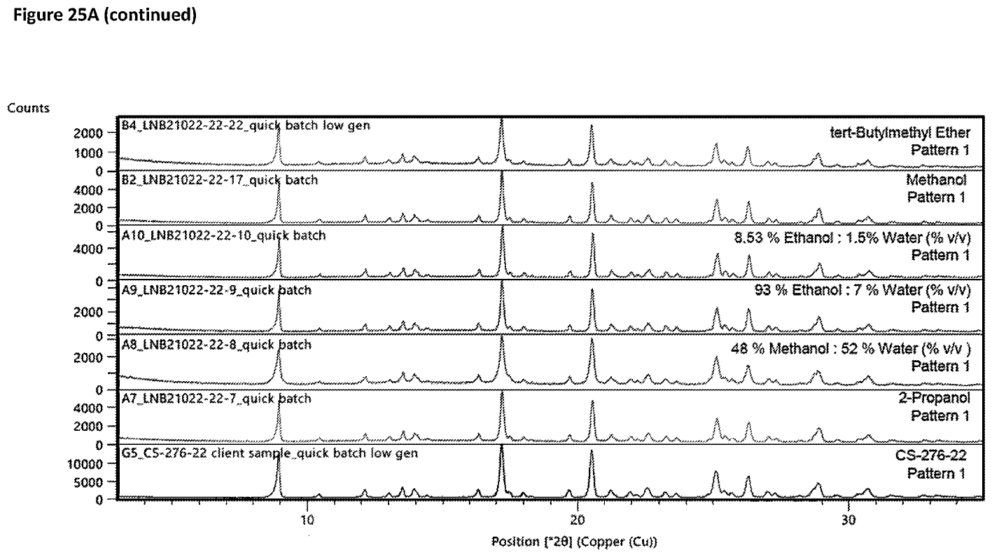

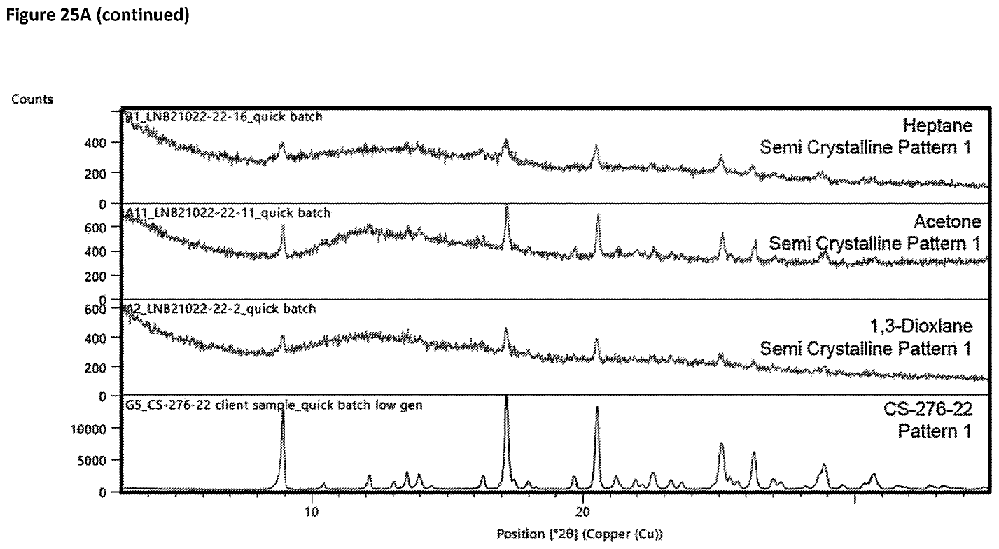

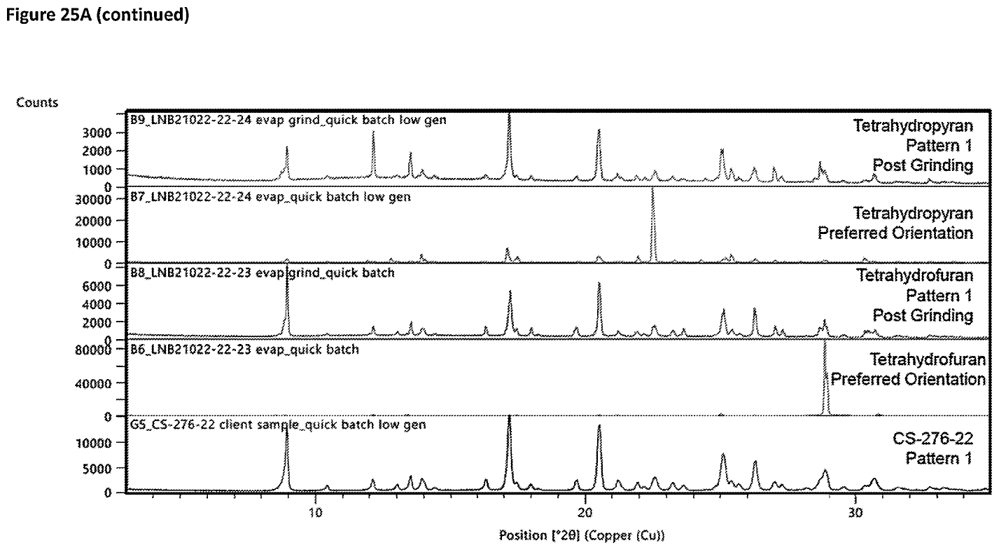

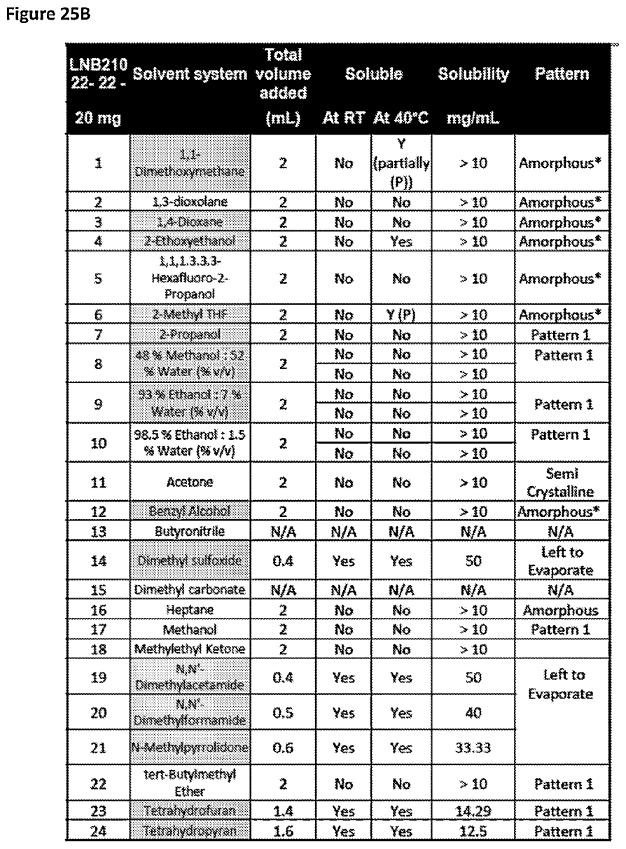

The results of the solvent solubility assessment are shown in FIG. 25A and summarised in FIG. 25B. The results indicate that all solids isolated from 2-propanol, 48% Methanol:52% Water (% v/v), 93% Ethanol:7% Water (% v/v), 98.5% Ethanol:1.5% Water (% v/v), Methanol and tert-Butylmethyl Ether returned to Pattern 1, with the exception of 1,1-Dimethoxymethane, 1,4-Dioxane, 2-Ethoxyethanol, 1,1,1,3,3,3-Hexafluoro-2-Propanol, 2-Methyl THF and Benzyl Alcohol, which all remained amorphous. High boiling point solvents, DMSO, DMA, DMP and NMP were left to continue evaporating. Solids isolated from evaporation of the clear solution of THF and THP showed preferred orientation, but later returned to Pattern 1 post grinding.

Preferred orientation is a commonly observed issue in XRPD experiments which may occur depending on the crystal size/morphology being analysed. In a normal experiment the crystalline powder has a distribution so that each face of the crystal is analysed, which gives ultimately an accurate diffractogram with peak intensities that correspond to the atoms(electrons) on each crystalline plane. However, when preferred orientation occurs, only specific planes of a crystal are observed. For example, needles tend to align themselves more like a packet of cigarettes than a random distribution. Thus, certain crystal planes are more exposed to the X-ray radiation than others. Preferred orientation may therefore sometimes result in diffractograms where only one peak is observed, or the appearance of peaks that would normally not be observed. Gentle grinding of samples may thus be used to minimise these potentially misleading effects.

Based on the solvent solubility assessment results, the 16 solvent systems shown in Table 6 were selected for the polymorph screening experiments:

TABLE 6
Solvent systems selected for polymorph screening experiments
Solvent system

1,1-Dimethoxymethane
1,3-dioxolane
1,4-Dioxane
2-Ethoxyethanol
1,1,1,3,3,3-Hexafluoro-2-
Propanol
2-Methyl THF
2-Propanol
48% Methanol: 52%
Water (% v/v)
93% Ethanol: 7% Water
(% v/v)
Benzyl Alcohol
Dimethylsulfoxide
N,N′-Dimethylacetamide
N,N′-Dimethylformamide
N-Methylpyrrolidone
Tetrahydrofuran
Tetrahydropyran

The following experiments were carried out to identify additional farudodstat polymorphs: solvent drop grinding, fast cooling, fast evaporation at 50° C. and 100° C. (thermal cycling), anti-solvent addition at ambient (room temperature) and at high temperature (50° C.), and a 2-week slurry experiment at 40° C.

### Solvent Drop Grinding

16×20 mg of the farudodstat DSP polymorph sample was weighed into 2 mL bead mill vials and was milled for 1.5 hrs for amorphization. Next, 5 μL of appropriate solvents (see Table 6) were added to each sample. The samples were then bead milled using the following procedure:
1. 2 stainless steel beads were added to each vial.
2. 4500 rpm
3. 10×90 s cycles
4. 10 s hold between each cycle
5. Repeated 4× to give 1 hr milling time

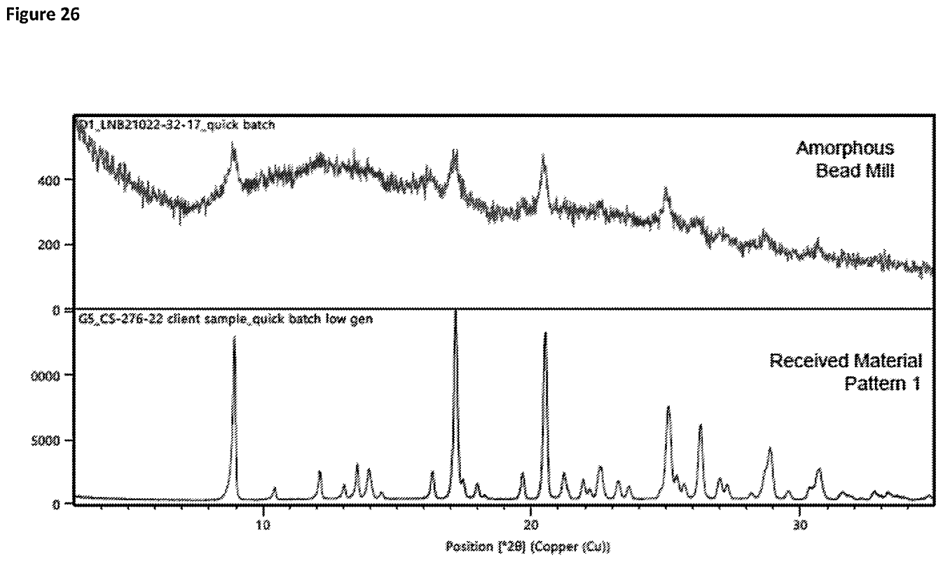

The solids post milling were first analysed by XRPD and compared with the farudodstat DSP polymorph to check for amorphous pattern. This checking step was also performed for the other experiments whenever bead milling was performed. A representative XRPD result is shown in FIG. 26. The XRPD results suggest that the bead milling of farudodstat generally gave less crystalline material. Similar XRPD patterns were observed for post bead milling checks performed in other experiments.

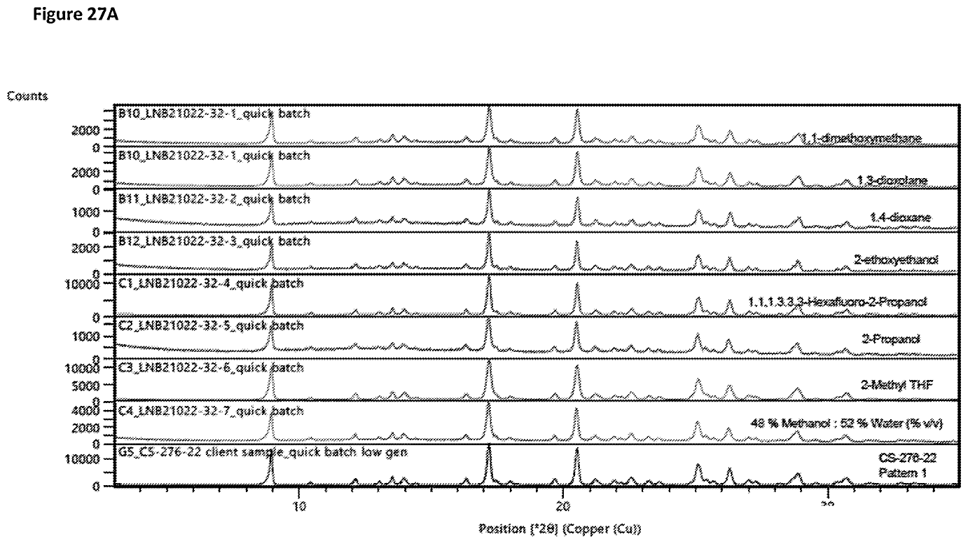

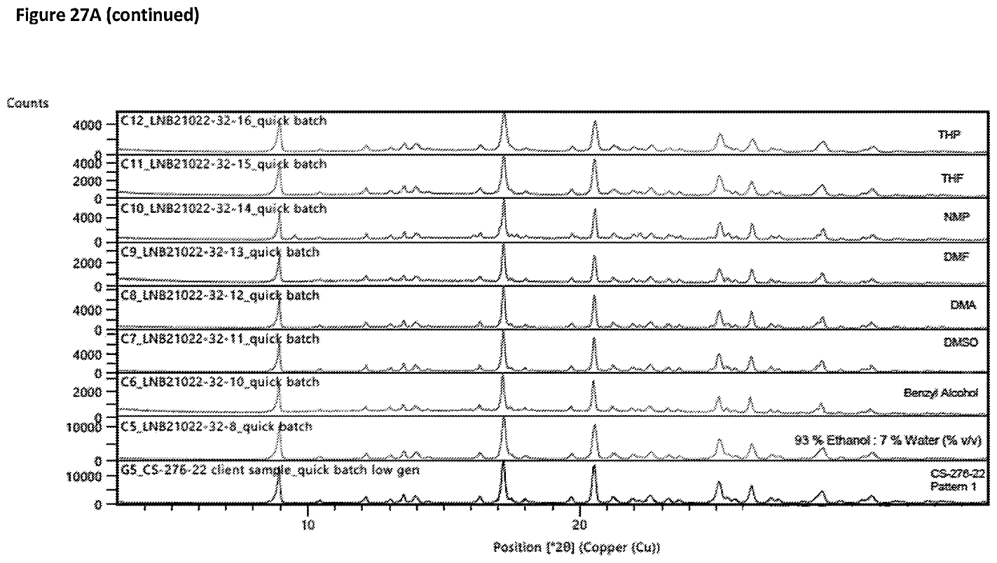

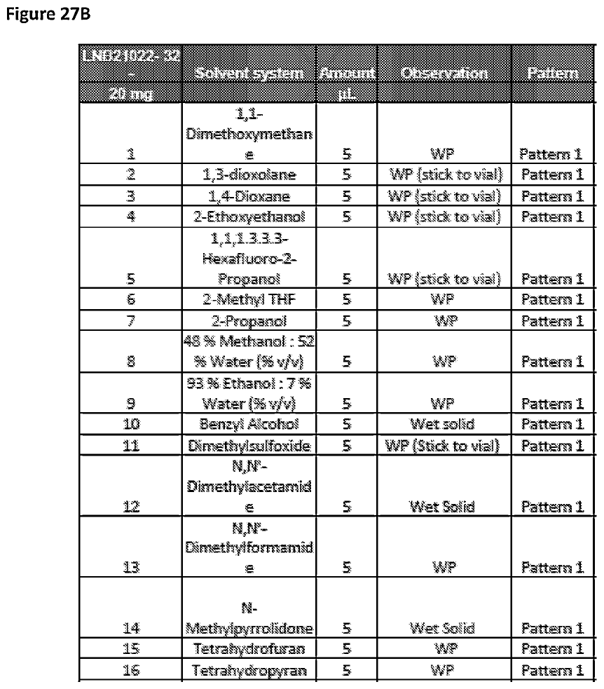

FIG. 27A shows the XRPD patterns from the solvent drop grinding experiment for the 16 solvent systems tested and FIG. 27B summarises the results.

The results indicate that the solids returned from solvent drop grinding from all 16 solvent systems returned to pattern 1.

### Thermal Cycling Experiment

16×20 mg of farudodstat sample was first weighed into 2 mL bead mill vials and was milled to prepare amorphous material. To all the 20 bead milled samples, appropriate solvents (see Table 6) were added in 100 μL aliquots until a mobile slurry was obtained without any dissolution observed. Between additions, the samples were stirred at 40° C. Samples were then stirred, and temperature cycled between 5-40° C. (0.1° C./min heating/cooling rate, 1 hour hold at each temperature) for ˜72 hours.

After 48 hours, samples were checked. If necessary, additional solids added. Non-mobile solid in solution samples were agitated using a spatula until a mobile slurry was obtained. Slurries were isolated by centrifugation (0.22 m nylon filters) and the solids analysed by XRPD. Post XRPD analysis, the XRPD plate was dried at 40° C. under vacuum for 48 hours and the solids reanalysed by XRPD to give an indication of form stability.

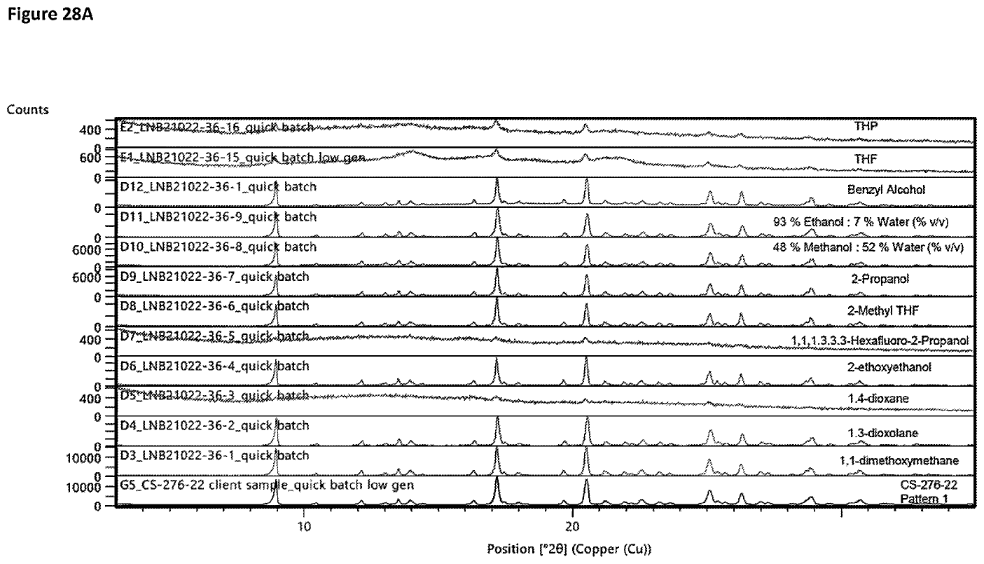

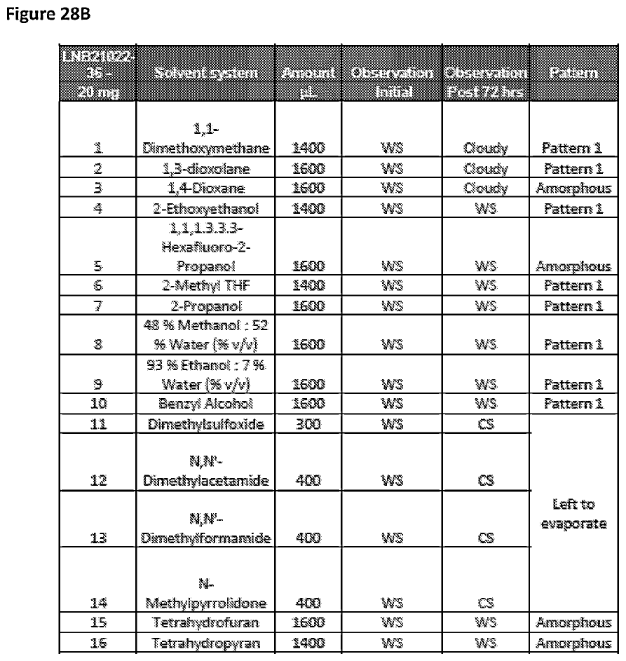

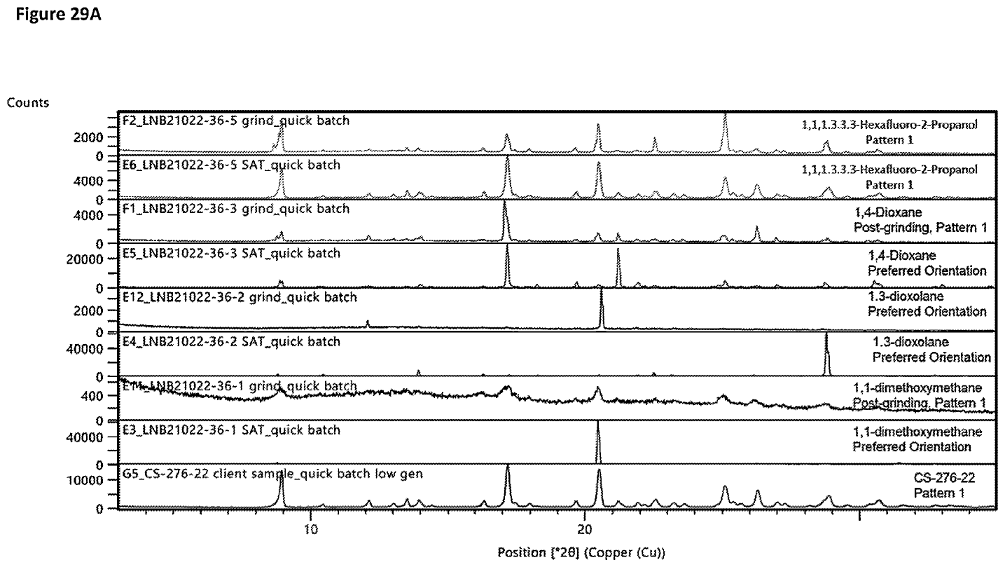

FIG. 28A shows the XRPD results from the thermal cycling experiment and FIG. 28B summarises the results.

High boiling point solvents such as DMSO, DMA, DMF and NMP were clear solutions and were left to evaporate. After isolation of solids, the saturates were allowed to evaporate for 72 hrs and any solids returned were further analysed by XRPD.

The results indicate that the solids returned from the thermal cycling experiment returned to Pattern 1, with the exception of 1,4-dioxane, THP and THF which remained semi-crystalline.

### Room Temperature Evaporation Experiment

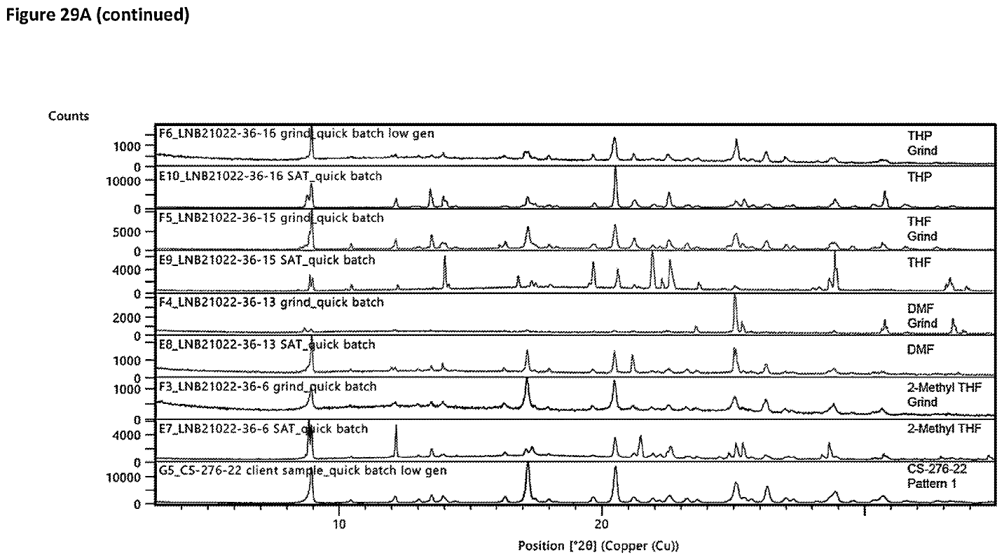

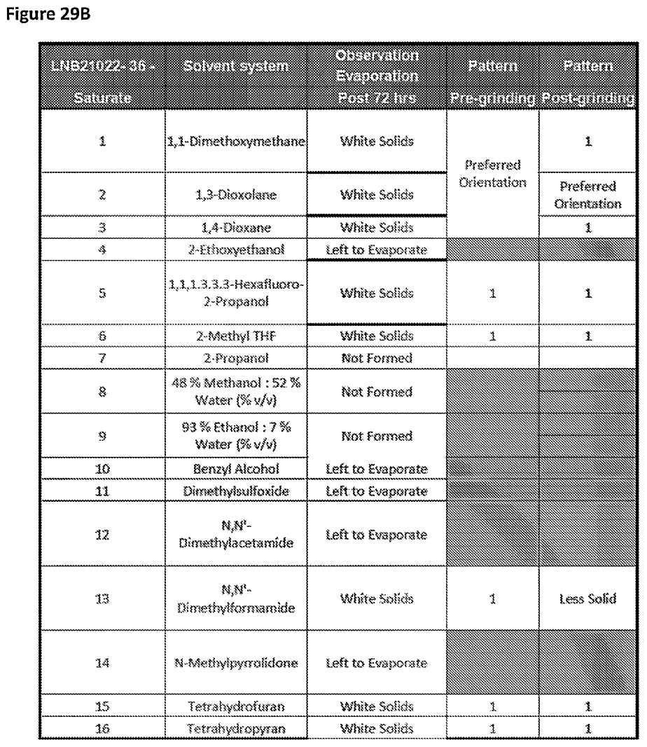

The saturate isolated from the previous thermal cycling experiment was left to evaporate at room temperature. Any solids returned from evaporation were analysed by XRPD. FIG. 29A shows the XRPD results from the thermal cycling experiment and FIG. 29B summarises the results.

The results indicate that solids obtained from room temperature evaporation of 1,1,1.3.3.3-Hexafluoro-2-Propanol, 2-Methyl THF, DMF, and THF and THP returned to Pattern 1. Solids returned from 1,1-Dimethoxymethane, 1,3-Dioxolane, and 1,4-Dioxane showed preferred orientation of Pattern 1 but returned to Pattern 1 after grinding.

### Fast Evaporation Experiment at 50° C.

16×20 mg of farudodstat sample was weighed into 2 mL bead mill vials and then milled to prepare amorphous material. To the bead milled sample, appropriate solvent was added in 100 μL aliquots until a mobile slurry was obtained without any dissolution observed. Between additions, the samples were stirred at 50° C. and 100° C. depending upon the boiling point of the solvent to dissolve the API in the solvent system. Samples were checked and slurries were isolated by centrifugation (0.22 m Nylon filters) and the saturates were put in an HPLC vials. Next, the vials were uncapped and were left to evaporate at 50° C. using a water bath. Finally, after the complete evaporation of solvent, the remaining solid were analysed by XRPD to check for appearance of any new patterns.

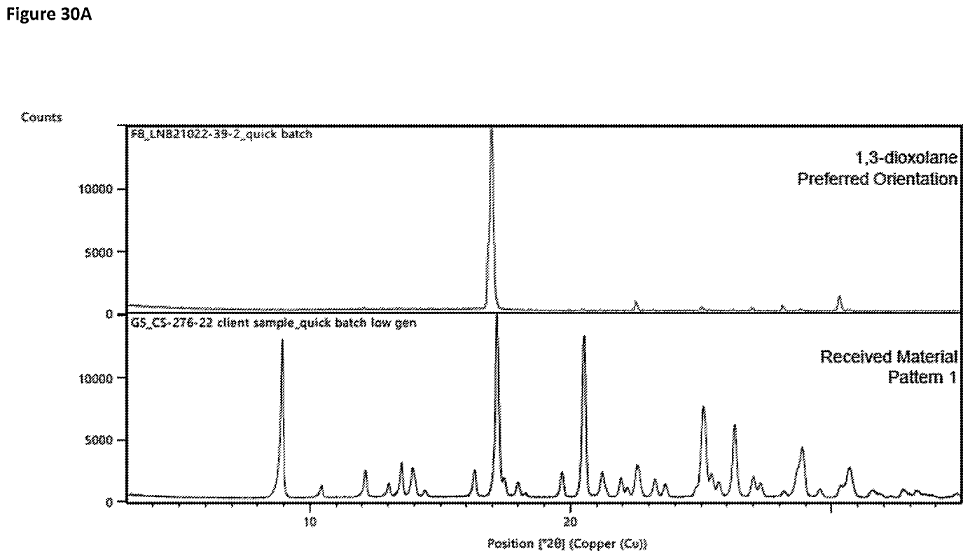

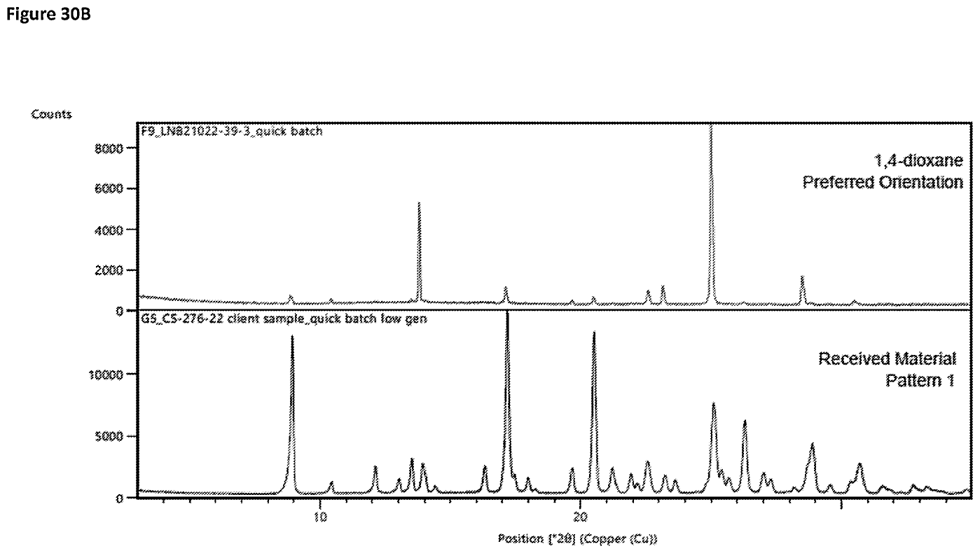

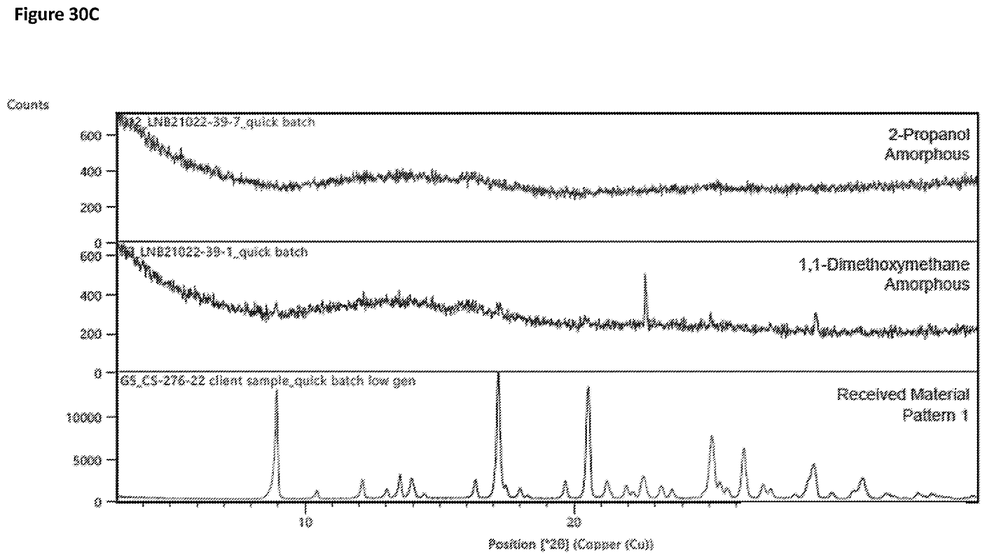

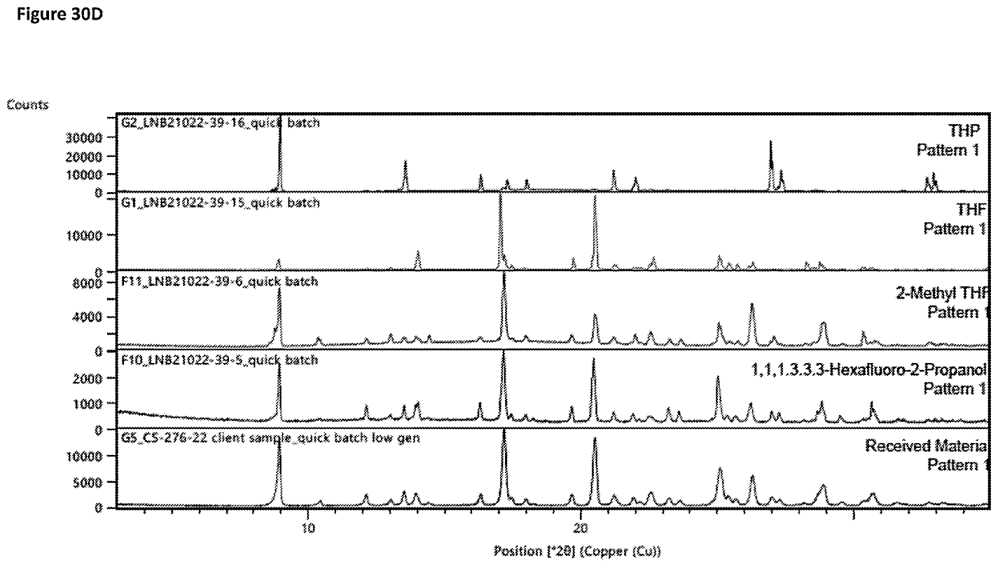

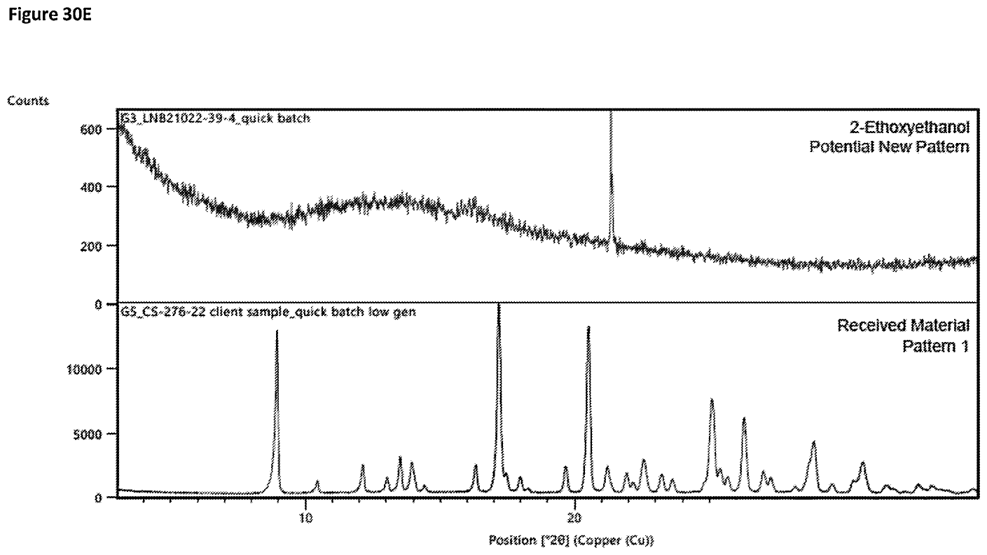

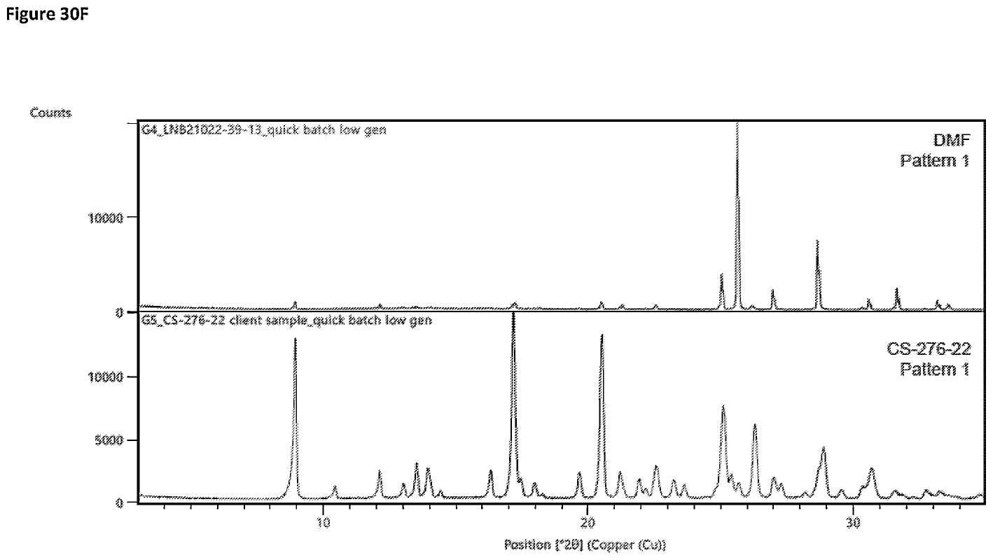

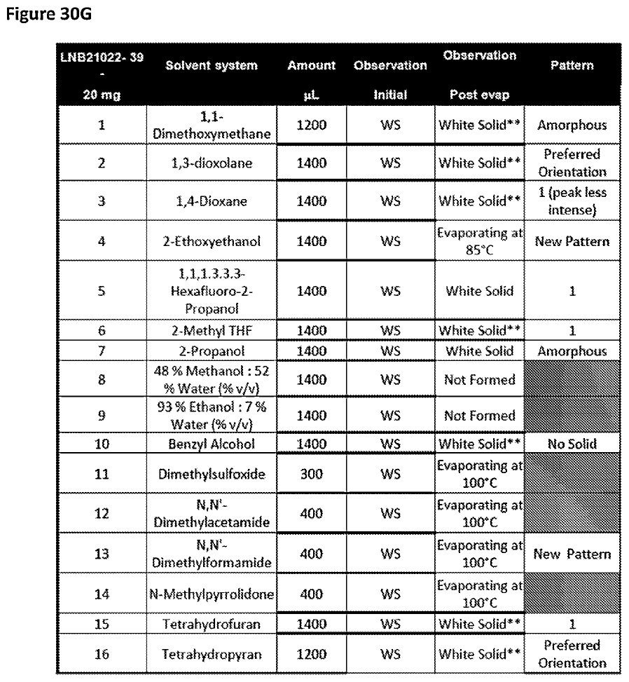

FIGS. 30A to F shows the XRPD results from the thermal cycling experiment and FIG. 30G summarises the results.

Solids returned post fast evaporation at 50° C. of saturate in 1,3-dioxlane, 1,4-dioxane showed preferred orientation. See FIGS. 30A and B. Solids returned post fast evaporation at 50° C. of saturate in 1,1-dimethoxymethane and 2-propanol showed amorphous pattern. Little solid was obtained from the 2-propanol sample. See FIG. 30C. Solids returned post fast evaporation at 50° C. of saturate in THP, THF,2-methyl THF and 1,1,1.3.3.3-Hexafluoro-2-Propanol returned to Pattern 1. See FIG. 30D. A potential new pattern was observed in 2-Ethoxyethanol with the appearance of a new peak at 21.1° 2θ. See FIG. 30E. Solids returned post fast evaporation at 50° C. of saturate in DMF showed pattern 1 with significant preferred orientation. See FIG. 30F. DMA, NMP and DMSO were still in solution.

### Fast Evaporation Experiment at 100° C. for High Boiling Point Solvents (DMSO, DMA, DMF and NMP)

20 mg×5 of farudodstat sample was weighed into 2 ml bead mill vials and milled to prepare amorphous material. To the bead milled sample, appropriate solvent was added in 100 μL aliquots until a mobile slurry was obtained without any dissolution observed. Between additions, the samples were stirred at 100° C. to dissolve the API in the solvent system. Samples were checked and slurries were isolated by centrifugation (0.22 μm Nylon filters) and the saturates were put in an HPLC vials. The vials were then uncapped and were left to evaporate at 100° C. using a sand bath. After the complete evaporation of solvent, the remaining solid were analysed by XRPD to check for appearance of any new patterns.

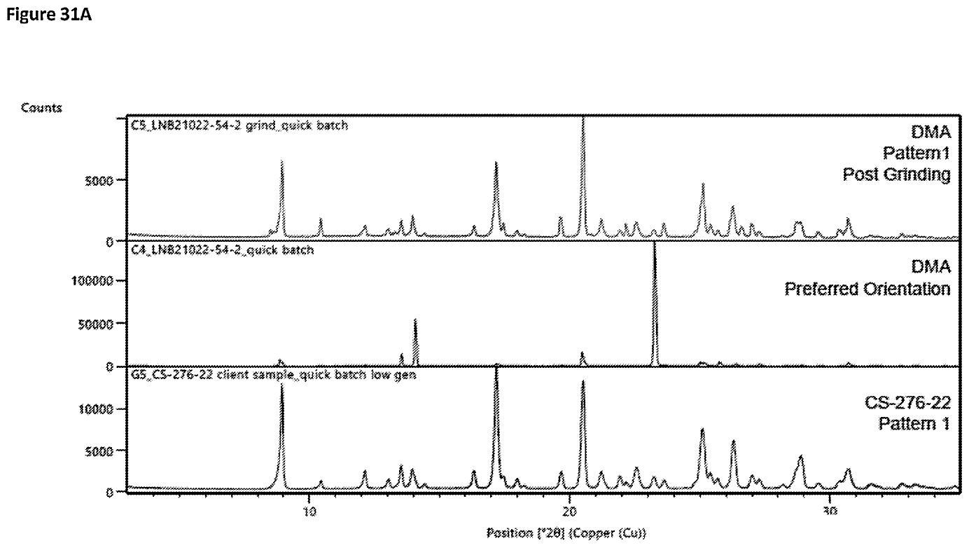

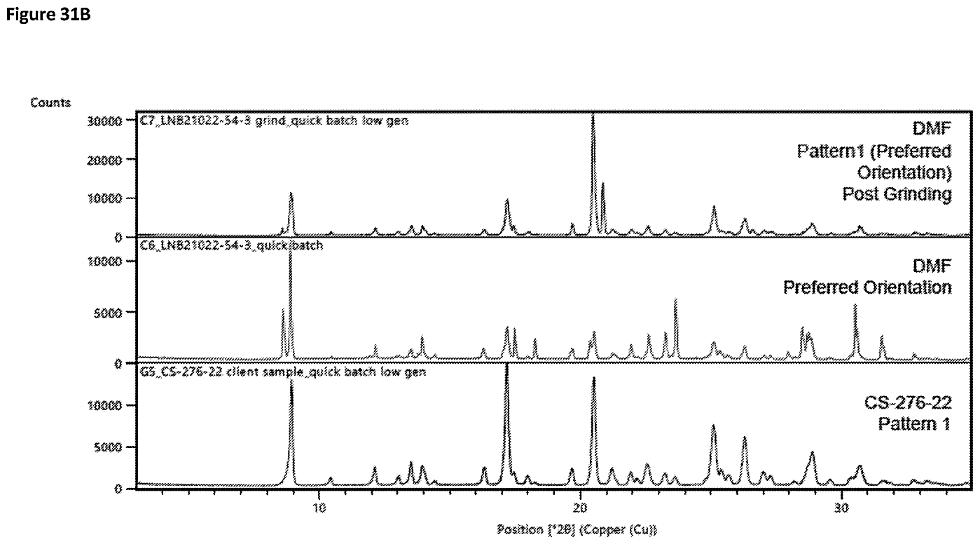

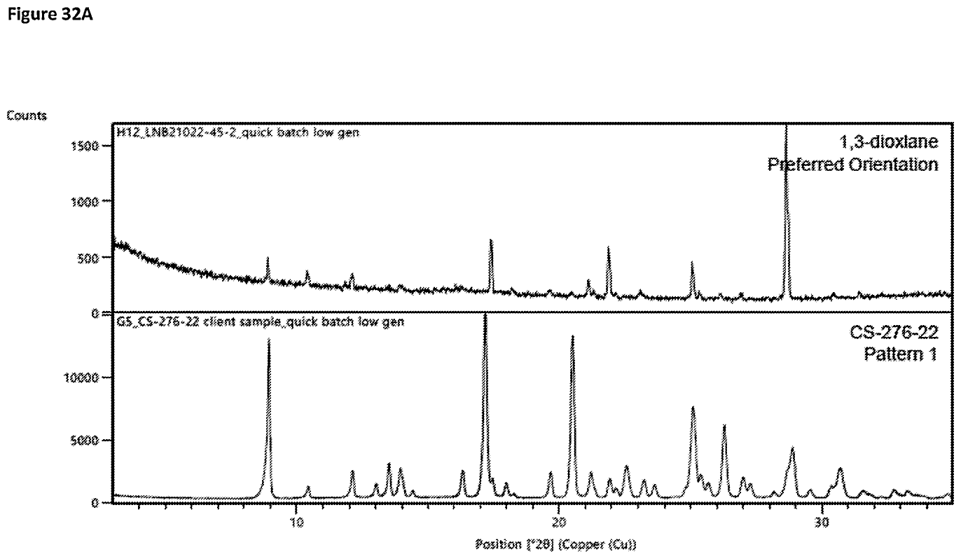

FIGS. 31A and B shows the XRPD results from the fast evaporation experiment at 100° C. and Table 7 below summarises the results.

TABLE 7
Summary of fast evaporation at 100° C. experiment results

| | | Amount | | | |
|---|---|---|---|---|---|
| | | of | | | |
| LNB21022- | | solvent | Observation post | | XRPD |
| 54- | Solvent | added | evaporation | XRPD | (Grind) |
| 1 | Dimethylsulfoxide | 300 | Ongoing | | |
| 2 | N,N′- | 350 | Light brown solid | Preferred | Pattern 1 |
| | Dimethylacetamide | | | Orientation | |
| 3 | N,N′- | 350 | Light brown | Preferred | Pattern 1 |
| | Dimethylformamide | | crystal | Orientation | |
| 4 | N- | 350 | Ongoing | | |
| | Methylpyrrolidone | | | | |

Pattern 1 = XRPD pattern for farudodstat DSP polymorph
Solids returned from fast evaporation of DMA showed preferred orientation, which however returned to Pattern 1 post-grinding. See FIG. 31A. Solids returned from fast evaporation of DMF showed preferred orientation, which however returned to Pattern 1 post-grinding. See FIG. 31B. DMSO and NMP saturates were left to continue evaporating.

### Fast-Cooling Experiment

20 mg×16 farudodstat sample was weighed into 2 ml bead mill vials and milled to prepare amorphous material. The appropriate solvent was added in 100 μL aliquots until a mobile slurry was obtained without any dissolution observed. Between additions, the samples were stirred at 50° C. and 100° C. depending upon the boiling point of the solvent to dissolve the API in the solvent system. Samples were checked and slurries were isolated by centrifugation (0.22 μm Nylon filters) and the saturates were put in the freezer for 72 hrs. The vials were checked for any solid precipitation. The solids were then isolated by centrifugation (0.22 m Nylon filters) and were analysed by XRPD.

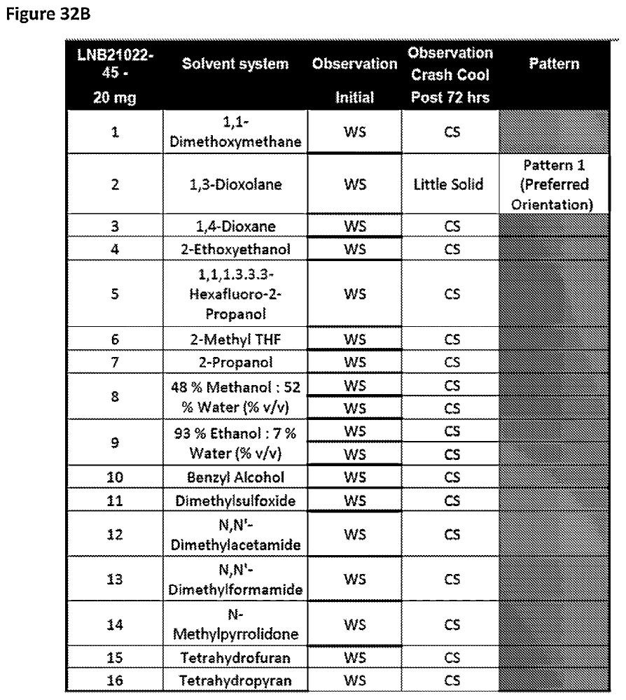

FIG. 32A shows the XRPD results from the fast-cooling experiment and FIG. 32B summarises the results.

The solids returned from crash cooling of 1,3-dioxolane showed preferred orientation of Pattern 1. No solids were obtained from fast cooling of the other 15 saturates.

### 2-Week Slurry Experiment

16×20 mg of farudodstat sample were weighed into 2 mL vials and was bead milled to prepare amorphous material. The following protocol was followed for milling:
2 stainless steel beads were added to each vial.
5000 rpm
10×90 s cycles
10 s hold between each cycle
Repeated ×6 to give 1.5 hr milling time

The milled samples were analysed by XRPD as usual to check for amorphous pattern. Next, the desired amount of appropriate solvent was added to the milled sample to make a white slurry. The vials were sealed with paraffin and isothermally stirred at 40° C. for two weeks. After 2 weeks, the slurries were observed and solids isolated were characterised by XRPD. Any clear solutions were left to evaporate at 100° C. and any solids returned were further analysed by XRPD.

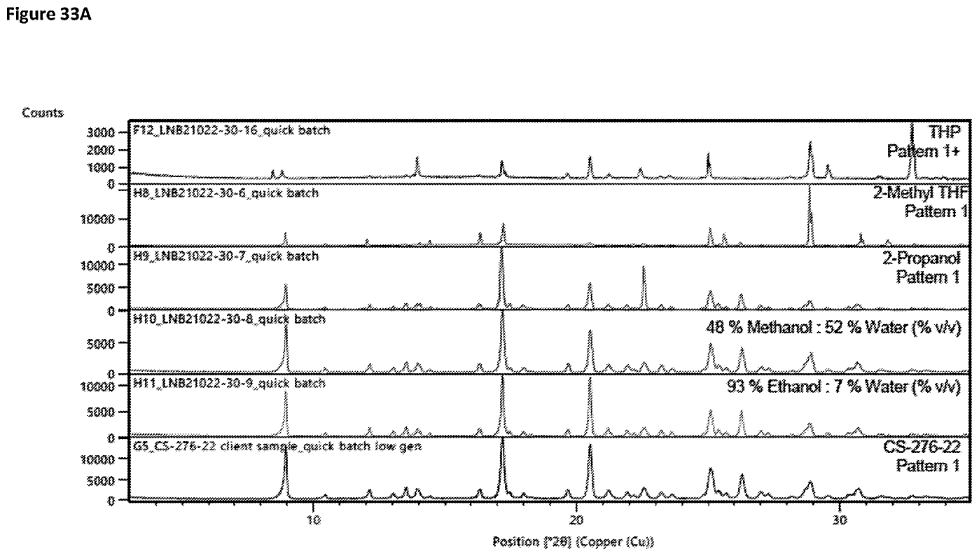

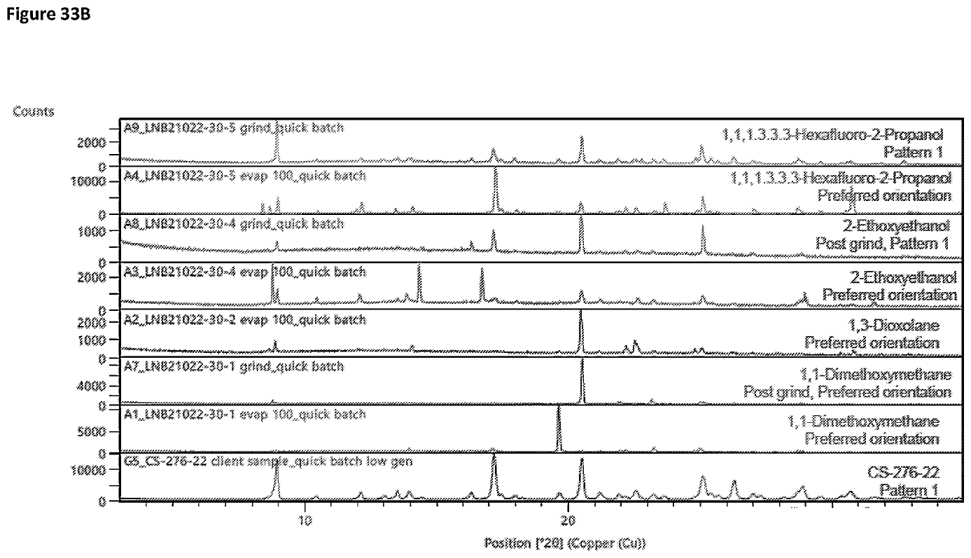

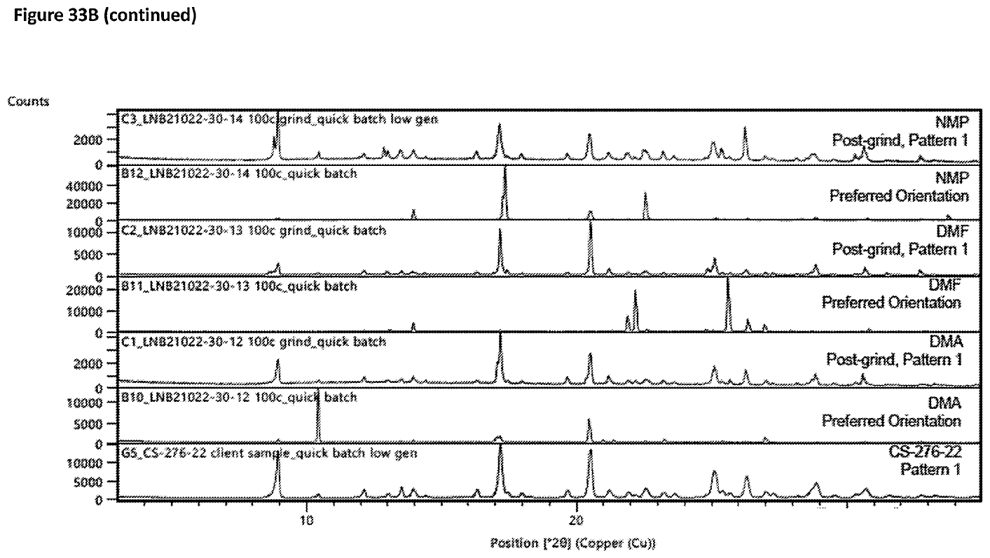

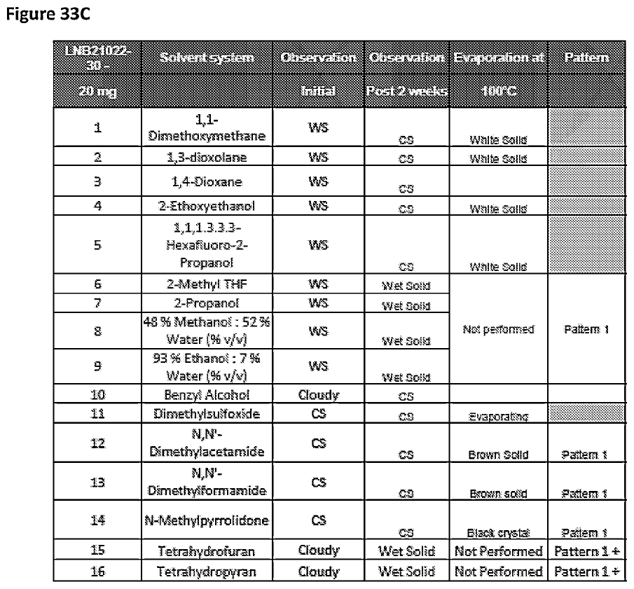

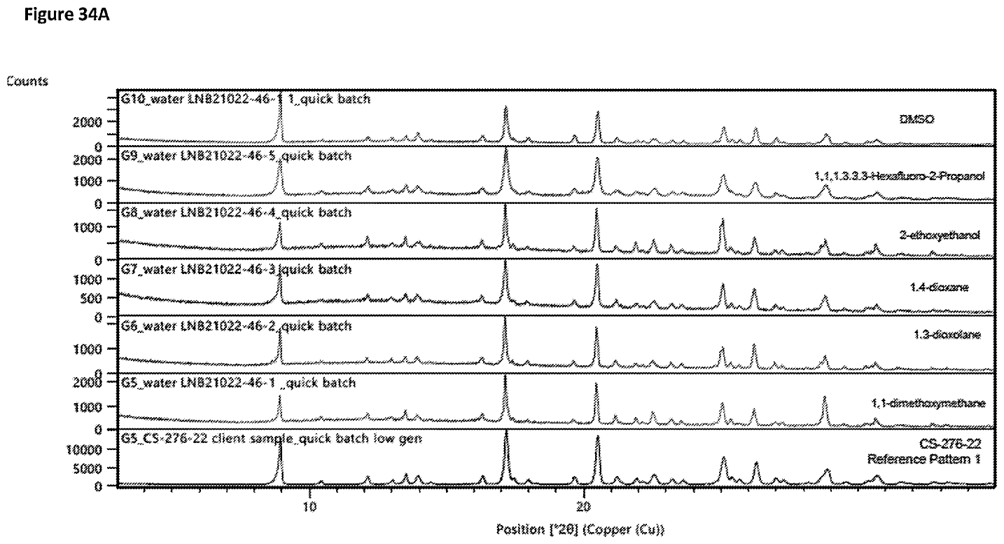

FIG. 33A shows the XRPD results from the 2-week slurry experiment. FIG. 33B shows the XRPD results from the 2-week slurry experiment for the samples subjected to fast evaporation at 100° C.

FIG. 33C summarises the results from the entire 2-week slurry experiment.

The results indicate that solids returned from 2-weeks slurry experiment of 93% Ethanol:7% Water (% v/v), 48% Methanol:52% Water (% v/v), 2-Propanol, and 2-Methyl THF returned to Pattern 1. Solids returned from THF and THP showed additional peaks with Pattern 1 at 8.1° 2θ (Pattern 1+).

Following fast evaporation at 100° C., solids obtained from 1,1-Dimethoxymethane, 1,3-Dioxolane, 2-Ethoxyethanol and 1,1,1.3.3.3-Hexafluoro-2-Propanol, DMA, DMF and NMP showed preferred orientation. However, post-grinding returned these to Pattern 1.

### Anti-Solvent Addition Experiment (Acetone, Heptane, MEK and Water)

20 mg×16 of farudodstat sample was weighed into 2 ml bead mill vials and milled to prepare amorphous material. The appropriate solvent was added in 100 μL aliquots until a mobile slurry was obtained without any dissolution observed. Samples were agitated at high temperature (40° C.). Samples were checked and slurries were isolated by centrifugation (0.22 μm Nylon filters) and the saturates were divided into 4 different vials. Water, MEK, Acetone and Heptane as anti-solvent was added to each vial at room temperature to check for any solid precipitation. The solids were then isolated by centrifugation (0.22 m Nylon filters) and were analysed by XRPD.

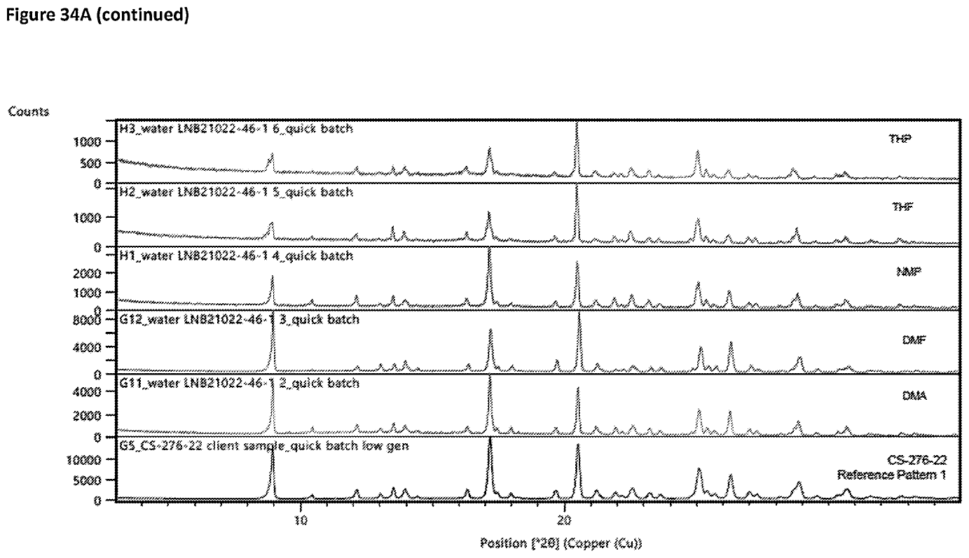

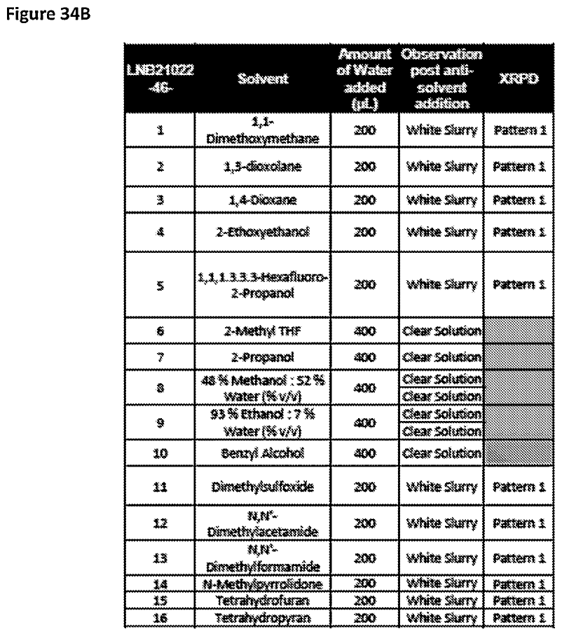

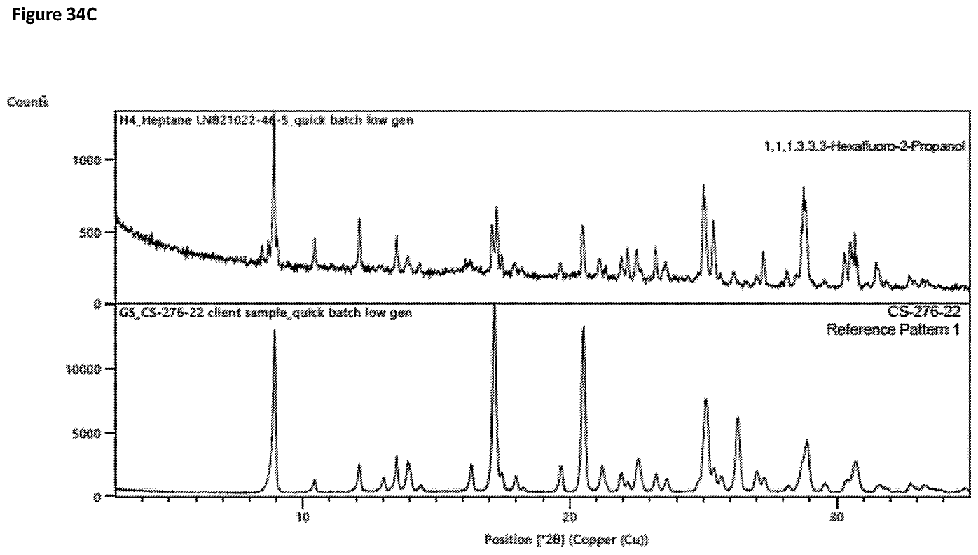

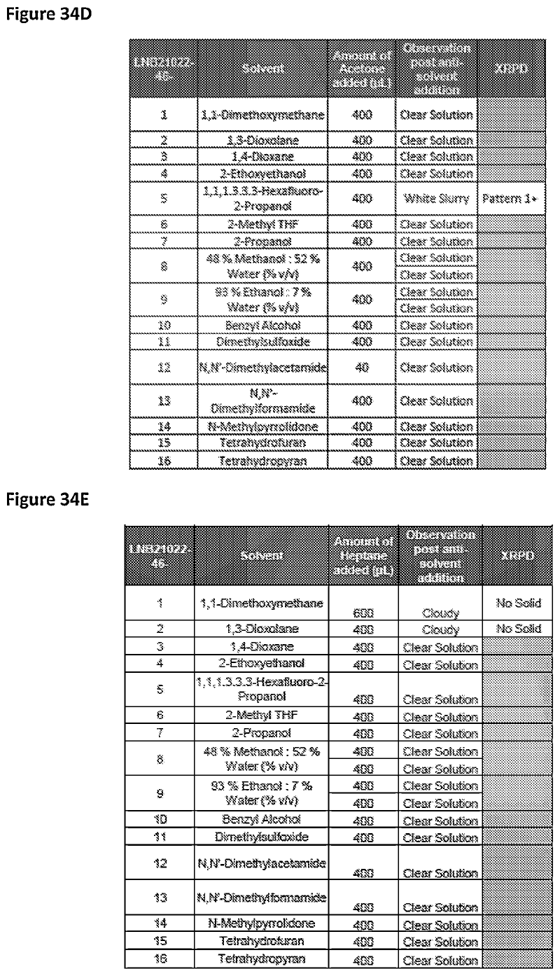

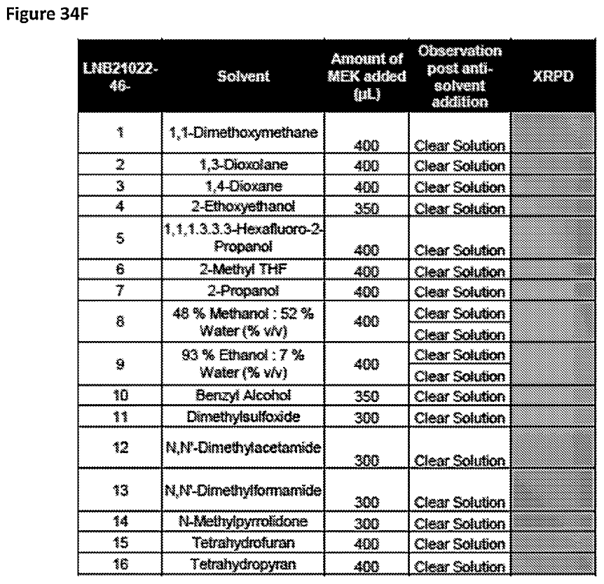

The results are shown in FIGS. 34A and C and summarised in FIGS. 34B and 34D to F.

The solids returned from water as an anti-solvent addition returned to Pattern 1 with the exception of 2-Methyl THF, 2-Propanol, 48% Methanol:52% Water (% v/v), 93% Ethanol:7% Water (% v/v) and Benzyl Alcohol, which remained as clear solutions. See FIG. 34A.

The solids returned from acetone as an anti-solvent addition for 1,1,1.3.3.3-Hexafluoro-2-Propanol returned to Pattern 1 with additional peaks which may be due to splitting of peaks because of preferred orientation. See FIG. 34C.

Cloudy solution was observed on heptane addition in 1,1-dimethoxymethane and 1,2-dioxolane. No solid precipitation was observed on heptane addition for any of the other 14 solvent systems. No solid precipitation was observed on MEK addition for any of the 16 solvent systems tested.

### Anti-Solvent Addition+Fast-Cool Experiment

All the clear solutions obtained from the previous anti-solvent addition experiment were put in the freezer for crash-cooling. After 72 hrs the samples were checked and solid precipitates were isolated by centrifugation (0.22 m Nylon filters) before reanalysis by XRPD.

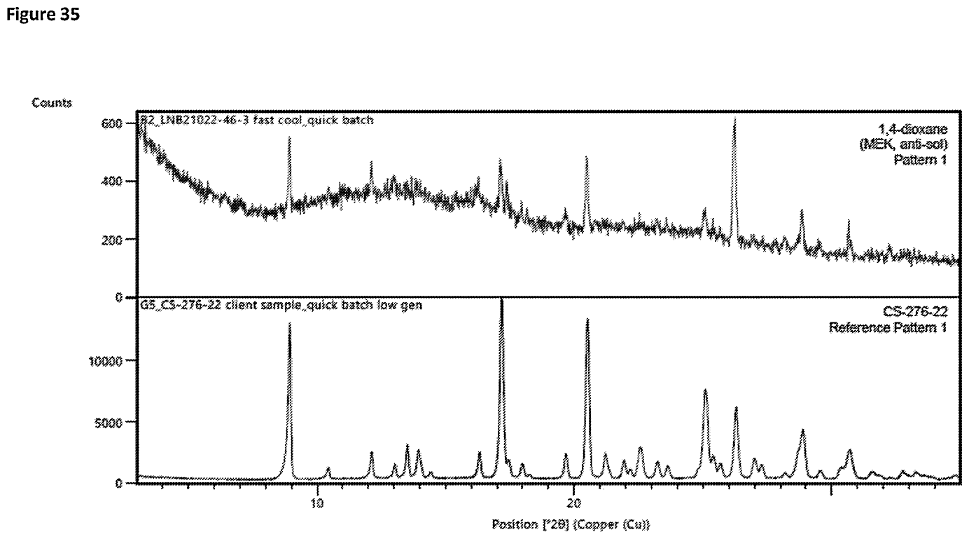

Only the sample with MEK as anti-solvent in 1,4-dioxane produced a white solid post-crash cooling and was analysed by XRPD. The XRPD result is shown in FIG. 35 and indicates that the sample had an XRPD pattern consistent with Pattern 1. All the other fast-cooled samples remained as clear solutions.

### Anti-Solvent Addition at High Temperature (50° C.)

20 mg×16 of farudodstat sample was weighed into 2 ml bead mill vials and milled to prepare amorphous material. The appropriate solvent was added in 100 μL aliquots until a mobile slurry was obtained without any dissolution observed. Samples were agitated at high temperature (40° C.). Next, the samples were checked and slurries were isolated by centrifugation (0.22 m Nylon filters) and the saturates were transferred into HPLC vials. Water as an anti-solvent was added to each vial at 50° C. to check for any solid precipitation. The solids were then isolated by centrifugation (0.22 m Nylon filters) and were analysed by XRPD.

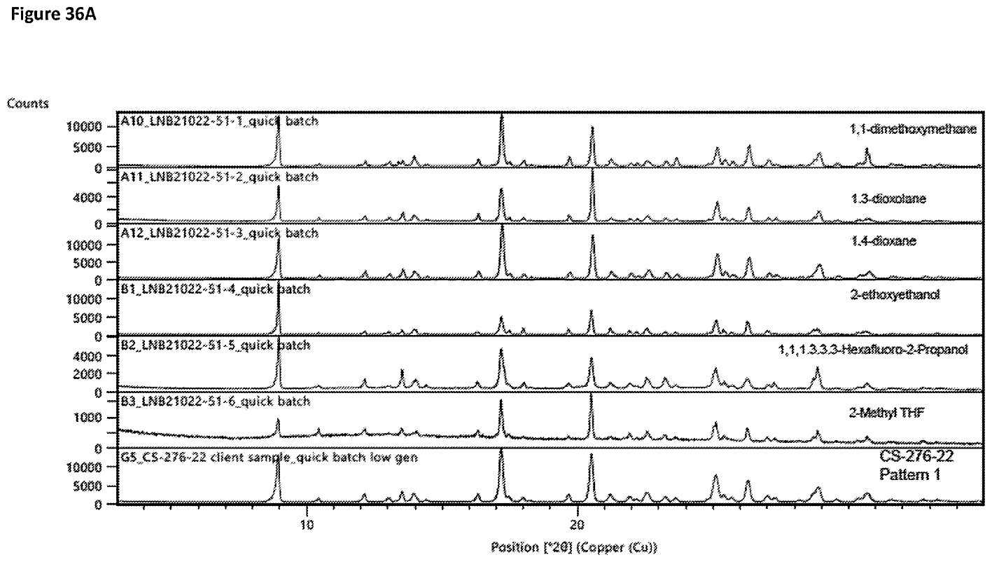

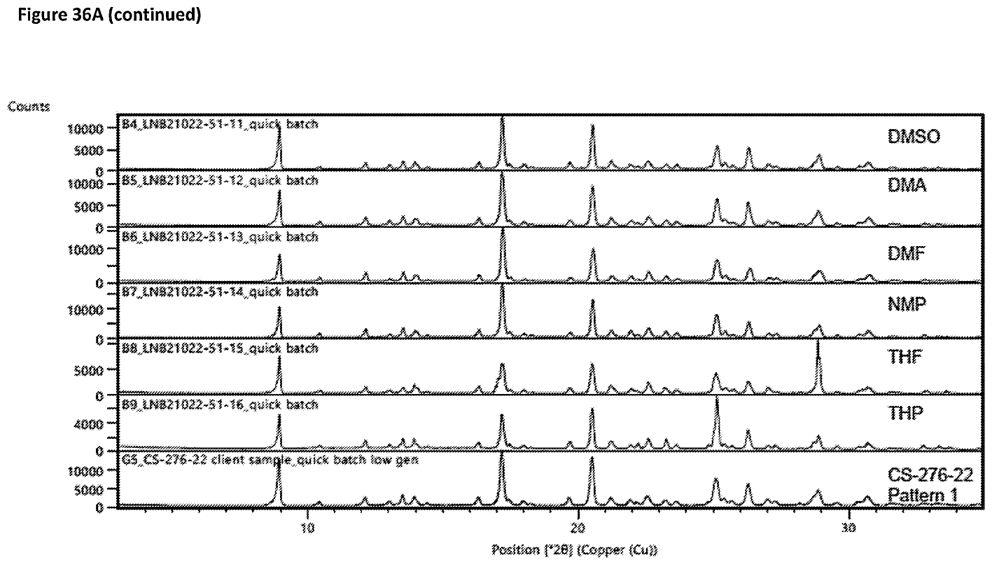

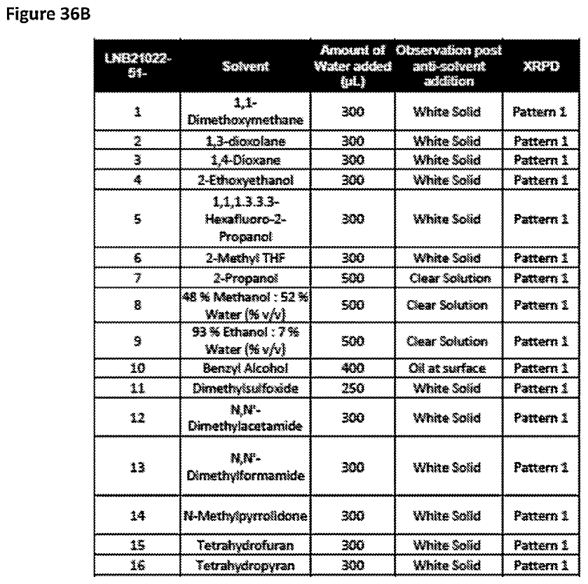

The results are shown in FIG. 36A and are summarised in FIG. 36B. The results indicate that the solids obtained from water addition to all the 16 saturates at 50° C. returned to Pattern 1.

### Conclusions

The majority of the solids returned from the solvent systems tested produced a XRPD pattern consistent with that of the farudodstat DSP polymorph (Pattern 1). Thus, the above experiments provide further evidence that the presently disclosed farudodstat DSP polymorph is highly stable.

### Example 9—Characterisation of Farudodstat Polymorphs

Solids returned from THF and THP in the 2-week slurry experiments showed additional XRPD peaks with Pattern 1 at 8.1° 2θ (Pattern 1+). In view of this interesting observation, the 2-week slurry experiments were repeated. As a result of the experiments, two new farudodstat polymorphs were identified:

Farudodstat polymorph obtained by crystallisation from 1,1,1.3.3.3-hexafluro-2-propanol (HIFP)

500 μL 1,1,1.3.3.3-Hexafluoro-2-Propanol was added to 20 mg of amorphous farudodstat DSP polymorph in a 1.5 mL of HPLC vial equipped with a magnetic stirring bar, resulting in formation of a mobile slurry. The slurry was isothermally stirred for 2 weeks at 40° C. An additional 20 mg of material was added to the slurry as it became a clear solution after one week. Solids were isolated by centrifugation using a 0.22 m Nylon filter, and were analysed without any further drying by XRPD, TGA and 1H NMR (proton nuclear magnetic resonance). Finally, the samples were dried at 40° C. for 4 hrs and then reanalysed by XRPD.

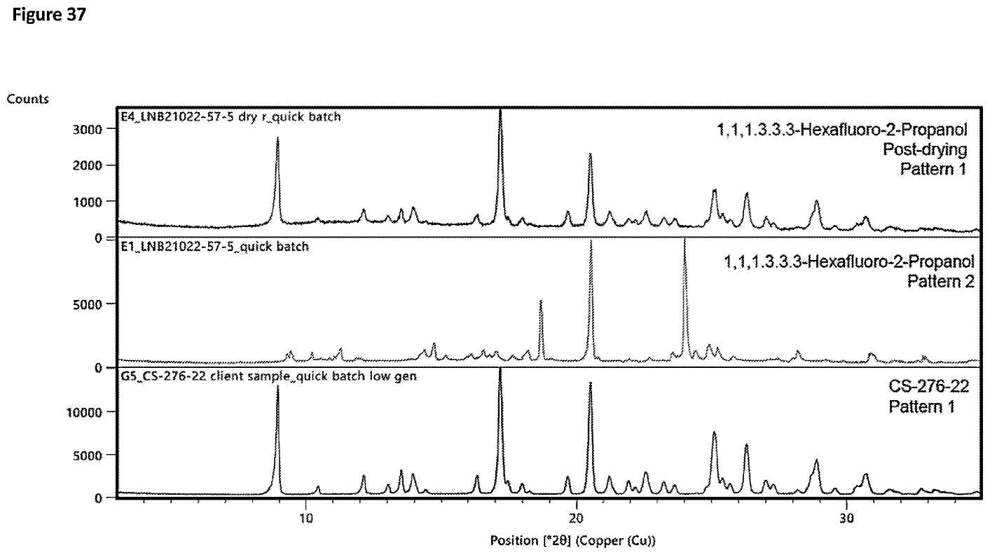

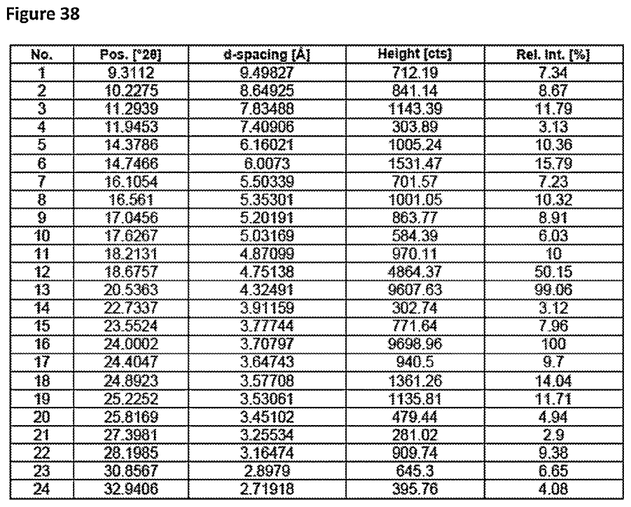

The XRPD results are shown in FIG. 37. As can be seen from FIG. 37, a clear new XRPD pattern (which we call Pattern 2) was observed for 1,1,1.3.3.3-Hexafluoro-2-Propanol. However, gentle drying at 40° C. for 4 hours returned the XRPD pattern back to that of the farudodstat DSP polymorph (Pattern 1). FIG. 38 shows the XRPD peak list for Pattern 2:

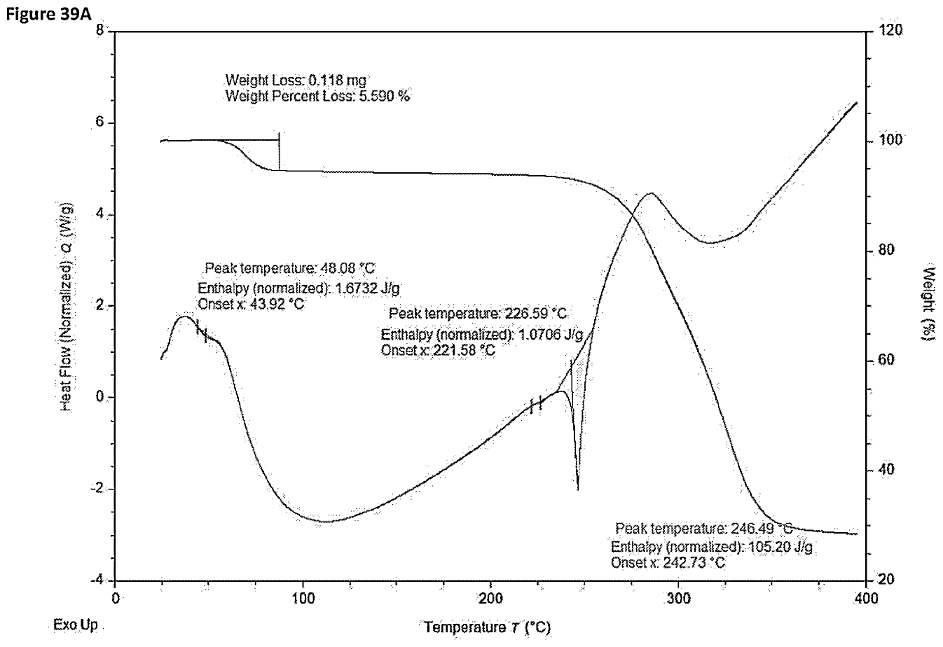

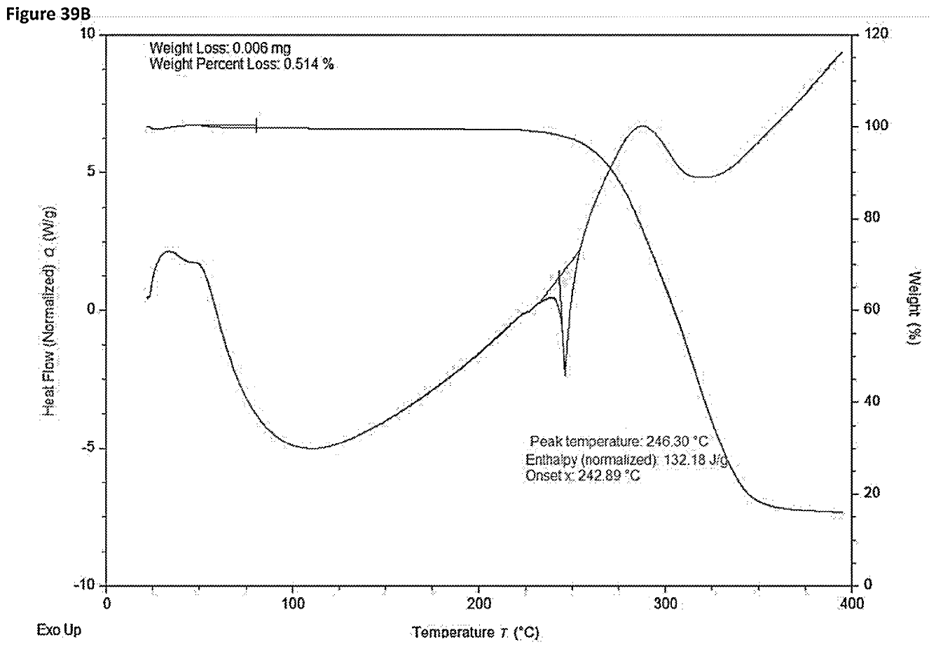

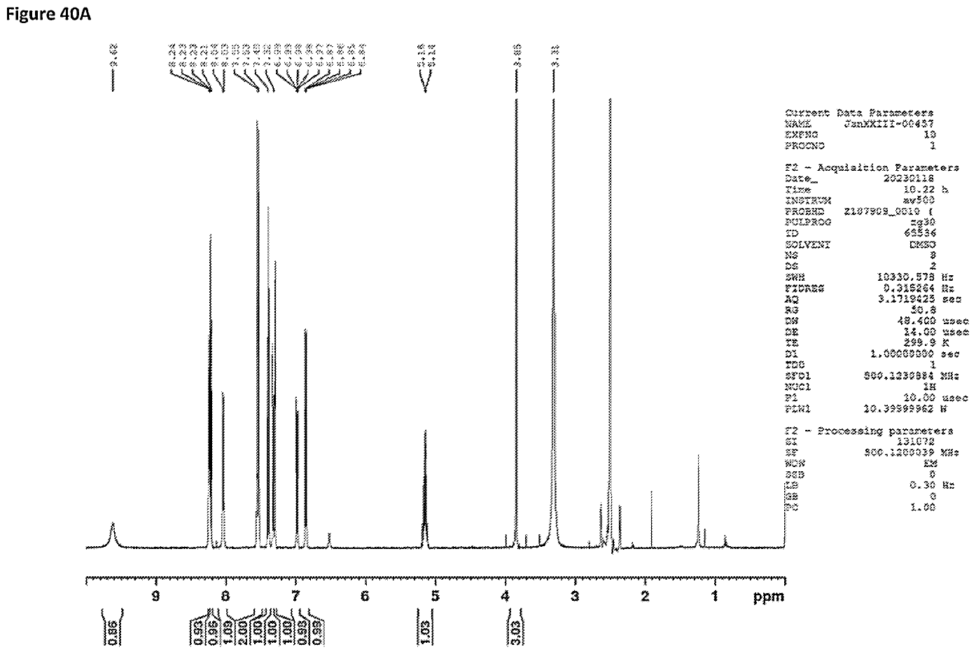

FIGS. 39A and B show the result of the TG analysis. The TG analysis indicates a mass loss of 0.118 mg (5.59%) which corresponds to 0.13 equivalents of Hexafluoro-2-Propanol. The simultaneous DSC analysis indicates an endothermic event consistent with the melt of Pattern 1. This suggests that the new 1,1,1.3.3.3-Hexafluoro-2-Propanol polymorph is a metastable form which readily returns to the farudodstat DSP polymorph after drying at 40° C. for 4 hours

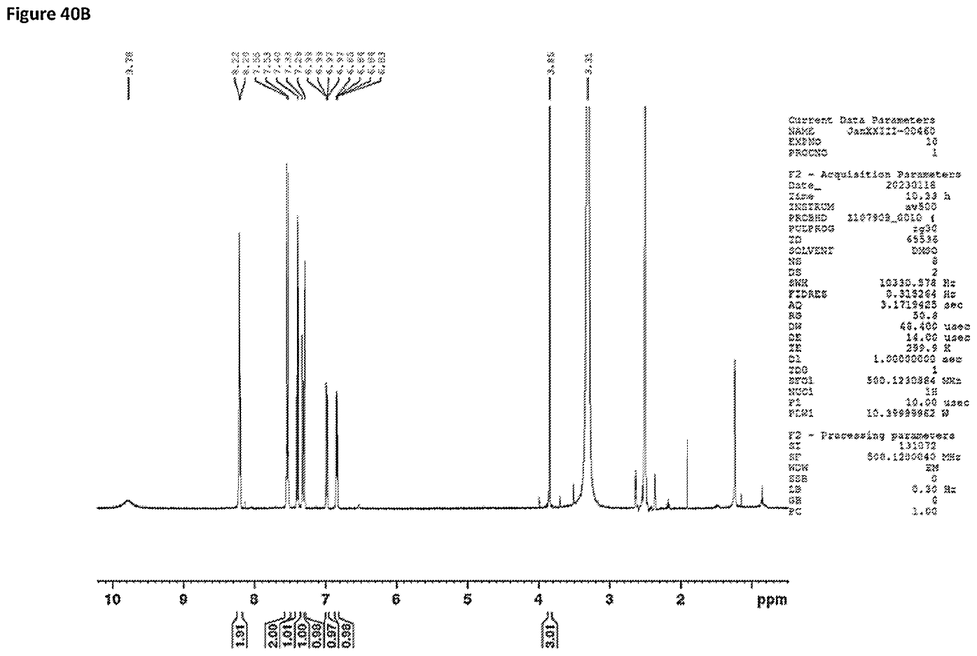

FIGS. 40A and B respectively show the 1H NMR spectra of the 1,1,1.3.3.3-Hexafluoro-2-Propanol polymorph before (wet) and after drying. The NMR spectra in FIG. 40A indicates that there is an additional peak at 5.15 ppm (methine proton of HFIP). The data overall also suggests that the 1,1.3.3.3-Hexafluoro-2-Propanol polymorph appears to be a mono-solvate.

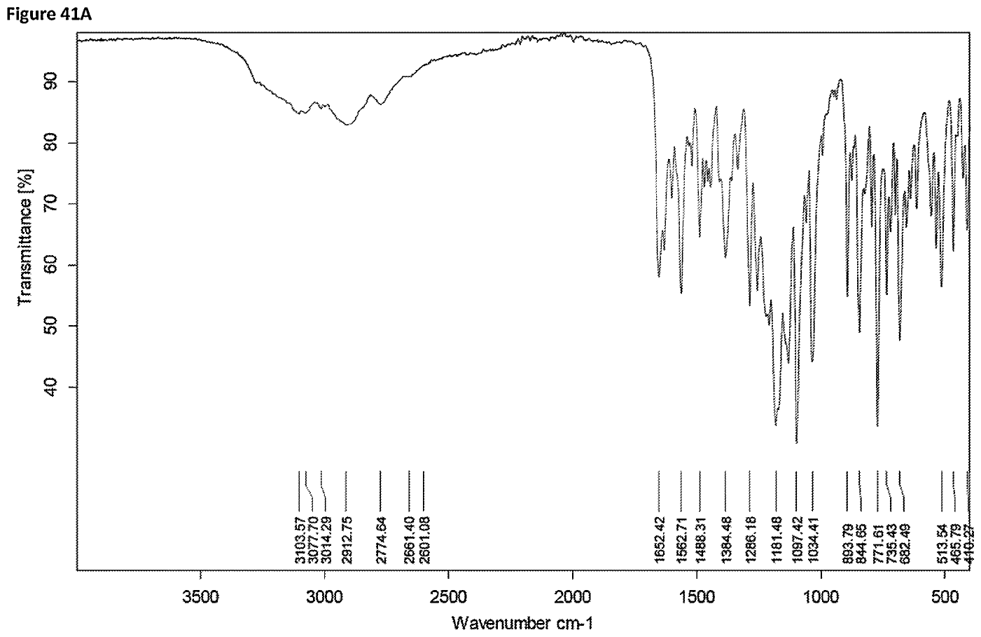

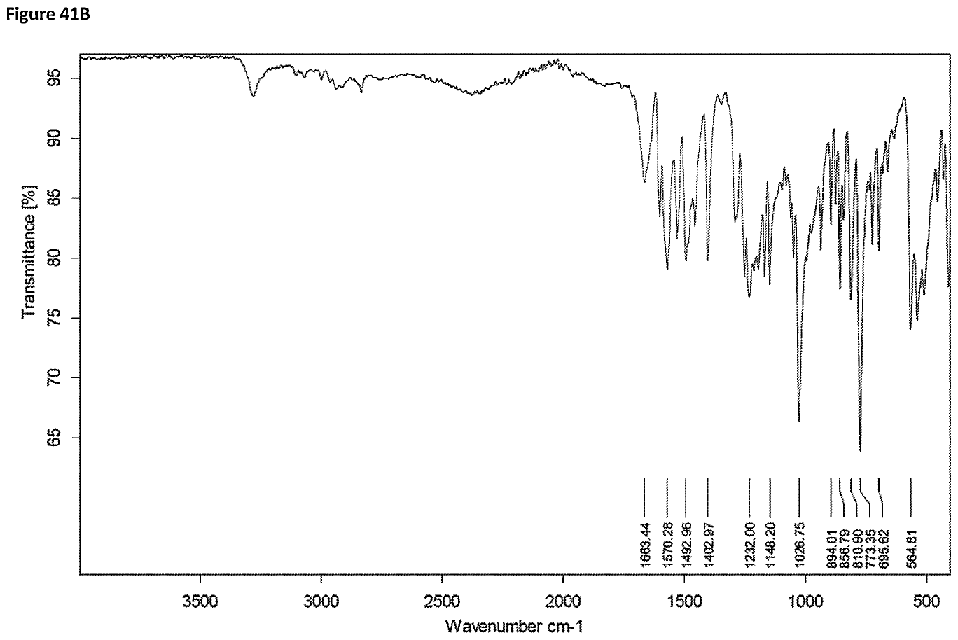

FIGS. 41A and B respectively show the FR-IR spectrum of the 1,1,1.3.3.3-Hexafluoro-2-Propanol polymorph vs the FR-IR spectrum of the farudodstat DSP polymorph. As can be seen, the FR-IR spectra is clearly different between the two samples.

The difference in solvent content between the TGA and NMR analysis can be accounted for at least in part by the nature of the analyses. In this respect, TGA uses a stream of nitrogen gas over samples during analysis. This appears to be sufficient to partially desolvate the 1,1,1.3.3.3-Hexafluoro-2-Propanol polymorph. In contrast, in NMR, the whole sample is dissolved in the NMR solvent and added to the NMR tube. Thus, the whole sample as well as the 1,1,1.3.3.3-Hexafluoro-2-Propanol solvent would be included in the subsequent analysis.

In summary, the data provides evidence that the polymorph obtained from 1,1,1.3.3.3-Hexafluoro-2-Propanol is a new but unstable polymorphic form of farudodstat.

Farudodstat Polymorph Obtained by Crystallisation from N-Methylpyrrolidone (NMP)

350 μL of N-Methylpyrrolidone (NMP) was added to 20 mg of amorphous farudodstat DSP polymorph in a 1.5 mL HPLC vial equipped with a magnetic stirring bar to prepare a mobile slurry. The resulting slurry was thermally cycled for 3 days between 5-40° C. (Ramp rate: 0.1° C./min with isothermal holds of 1 hour at 40° C. and 5° C.). The saturated solution was then evaporated at 100° C., before the solids were analysed by XRPD.

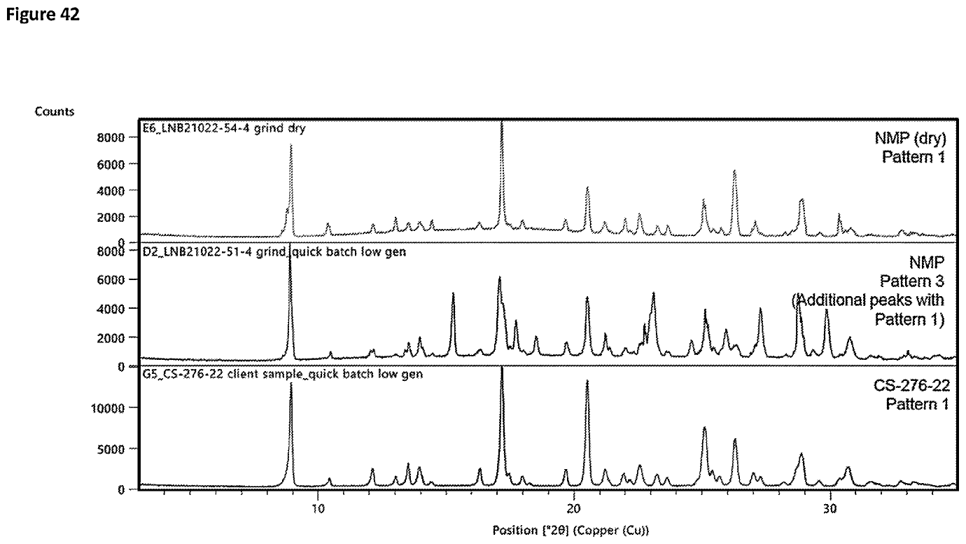

The XRPD results are shown in FIG. 42. The XRPD diffractogram of the evaporated solids (which we call Pattern 3) showed additional peaks alongside Pattern 1 at 15.28° 2θ, 17.73° 2θ 18.48° 2θ, 23.13° 2θ, 24.60° 2θ, 25.96° 2θ and 29.90° 2θ, suggesting that a mixture of the farudodstat DSP polymorph and another new polymorphic form was present Gentle grinding did not change the pattern observed. However, drying at 40° C. for 4 hours resulted in conversion of Pattern 3 back to Pattern 1 (i.e. the XRPD pattern for the farudodstat DSP polymorph).

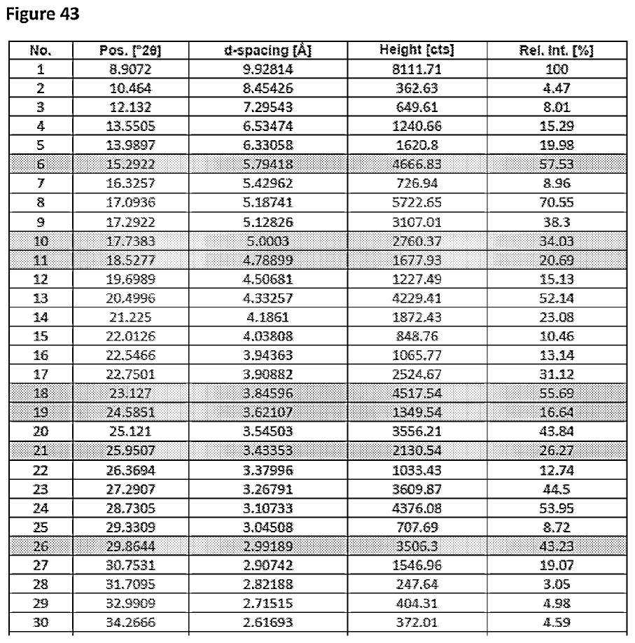

FIG. 43 shows the peak list for Pattern 3. The highlighted peaks are the additional peaks observed in addition to the Pattern 1 peaks.

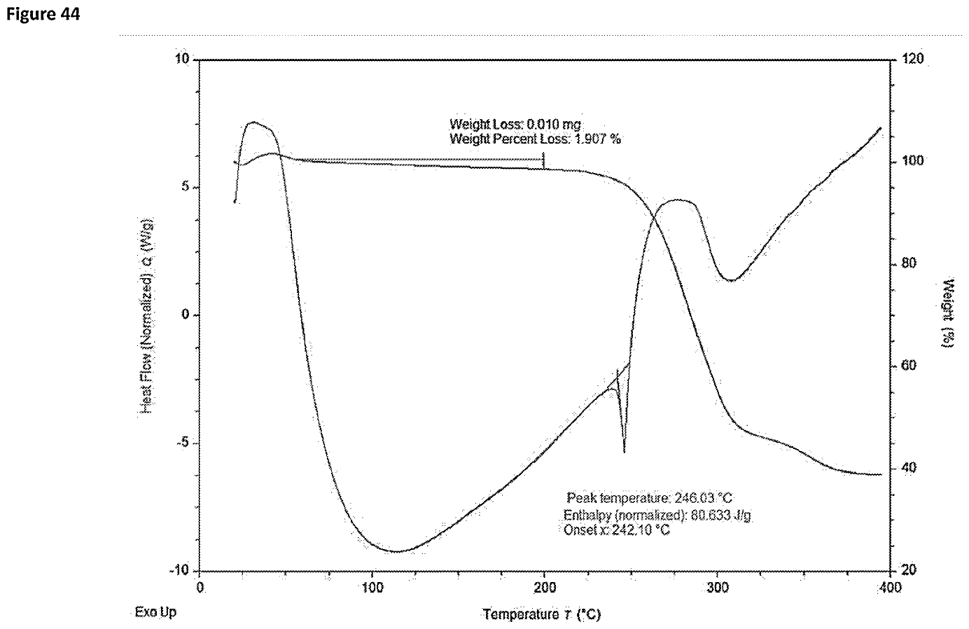

The results of the TG analysis are shown in FIG. 44. The analysis showed a mass loss of 0.048 mg from the onset of heating, which corresponds to 0.07 equivalents of NMP.

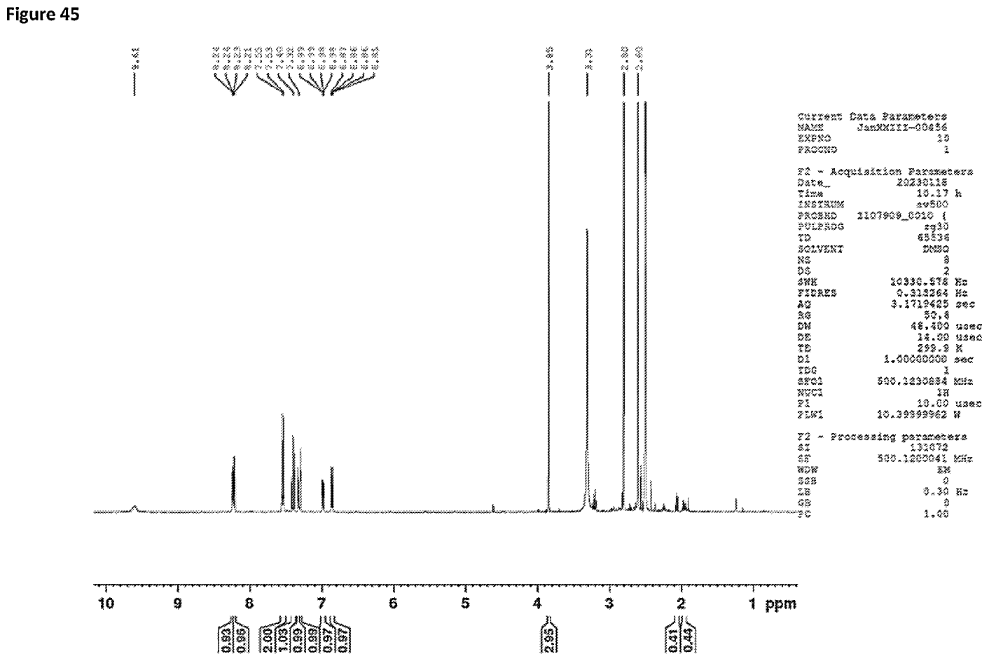

The results of the NMR analysis are shown in FIG. 45. The 1H NMR spectrum shows multiplets at 2.06 ppm and 1.96 ppm, which corresponds to 0.2 equivalents of NMP solvent present in the sample tested.

In summary, the above data demonstrates that a potential new farudodstat polymorph may be obtained by crystallisation from NMP. At the moment this new polymorph has not been isolated in a pure form, but was instead obtained as a mixture together with the farudodstat DSP polymorph.

However, given that gentle heating at 40° C. for 4 hours was sufficient to return Pattern 3 to Pattern 1, this new polymorph (like the 1,1,1.3.3.3-Hexafluoro-2-Propanol polymorph) is likely to be unstable/metastable.

### Conclusions

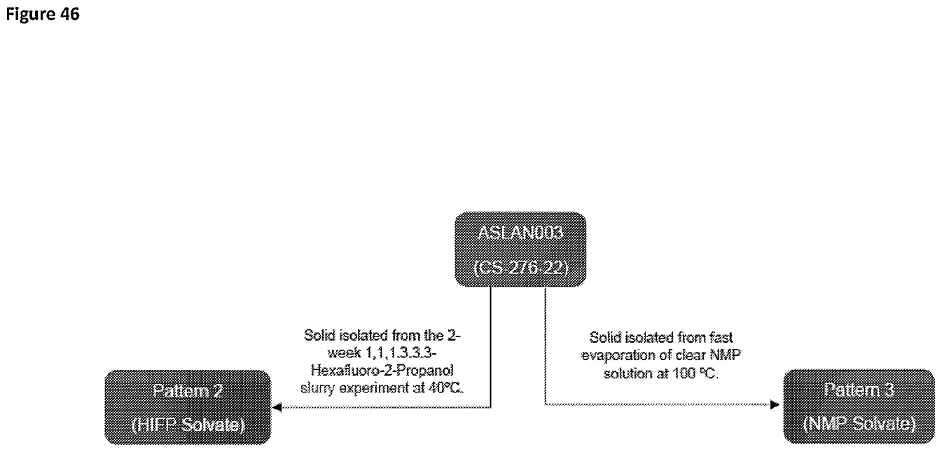

These experiments demonstrate that new farudodstat polymorphs may be obtained by crystallisation from the solvents 1,1,1.3.3.3-hexafluro-2-propanol and NMP (see FIG. 46 for summary diagram). The data however suggests that these new polymorphs have a high propensity to revert to the farudodstat DSP polymorph, thus further demonstrating the high stability of the farudodstat DSP polymorph that has been successfully produced and identified.

1-18. (canceled)

19. A polymorph of 2-(3,5-difluoro-3′methoxybiphenyl-4-ylamino)nicotinic acid, which is a DHODH inhibitor, characterised by a X-ray powder diffraction pattern comprising the following peaks in terms of degrees of 2-theta at approximately: 9.129±0.1, 17.375±0.1, 20.708±0.1, 25.274±0.1, 26.488±0.1 and 29.041±0.1.

20. The polymorph according to claim 19, wherein the X-ray powder diffraction pattern further comprises one or more of the following peaks selected from the group comprising: 13.686±0.1, 14.159±0.1, 19.900±0.1, 21.431±0.1, 22.750±0.1, 25.607±0.1 and 30.893±0.1.

21. The polymorph according to claim 19, wherein the X-ray powder diffraction pattern further comprises one or more of the following peaks selected from the group comprising: 12.313±0.1, 13.223±0.1, 18.167±0.1, 22.146±0.1, 23.418±0.1, 23.828±0.1, 25.885±0.1, 27.219±0.1 and 30.612±0.1.

22. The polymorph according to claim 19, wherein the X-ray powder diffraction pattern further comprises one or more of the following peaks selected from the group comprising: 10.645±0.1, 27.453±0.1, 29.762±0.1, 31.743±0.1 and 39.115±0.1.

23. The polymorph according to claim 19, wherein the X-ray powder diffraction pattern further comprises one or more of the following peaks selected from the group comprising: 14.580±0.1, 28.368±0.1, 32.950±0.1, 33.445±0.1, 33.800±0.1, 34.936±0.1, 35.497±0.1, 35.981±0.1, 37.678±0.1, 38.099±0.1 and 38.520±0.1.

24. The polymorph according to claim 19, wherein the X-ray powder diffraction pattern further comprises one or more of the following peaks selected from the group comprising: 12.313±0.1, 13.223±0.1, 13.686±0.1, 14.159±0.1, 16.477±0.1, 18.167±0.1, 19.900±0.1, 21.431±0.1, 22.146±0.1, 22.750±0.1, 23.418±0.1, 23.828±0.1, 25.607±0.1, 25.885±0.1, 27.219±0.1, 30.612±0.1 and 30.893±0.1.

25. The polymorph according to claim 19, wherein the X-ray powder diffraction pattern further comprises one or more of the following peaks selected from the group comprising: 10.645±0.1, 14.580±0.1, 27.453±0.1, 28.368±0.1, 29.762±0.1, 31.743±0.1, 32.950±0.1, 33.445±0.1, 33.800±0.1, 34.936±0.1, 35.497±0.1, 35.981±0.1, 37.678±0.1, 38.099±0.1, 38.520±0.1 and 39.115±0.1.

26. The polymorph according to claim 19, wherein the X-ray powder diffraction pattern further comprises the following peaks: 9.129±0.1, 10.645±0.1, 12.313±0.1, 13.223±0.1, 13.686±0.1, 14.159±0.1, 14.580±0.1, 16.477±0.1, 17.375±0.1, 18.167±0.1, 19.900±0.1, 20.708±0.1, 21.431±0.1, 22.146±0.1, 22.750±0.1, 23.418±0.1, 23.828±0.1, 25.274±0.1, 25.607±0.1, 25.885±0.1, 26.488±0.1, 27.219±0.1, 27.453±0.1, 28.368±0.1, 29.041±0.1, 29.762±0.1, 30.612±0.1, 30.893±0.1, 31.743±0.1, 32.950±0.1, 33.445±0.1, 33.800±0.1, 34.936±0.1, 35.497±0.1, 35.981±0.1, 37.678±0.1, 38.099±0.1, 38.520±0.1 and 39.115±0.1.

27. The polymorph according to claim 19, characterized by an X-ray powder diffraction pattern as shown in FIG. 2.

28. A composition comprising the polymorph according to claim 19 and a pharmaceutically acceptable excipient, diluent or carrier.

29. A method of treating a viral infection or an autoimmune disease comprising administering a therapeutically effective amount of the polymorph according to claim 19 to a subject in need thereof.

30. The method according to claim 29, wherein the viral infection is caused by a virus selected from the group comprising: dengue (such as serotypes 1, 2, 3 or 4), Zika, Chikungunya and SARS-CoV-2.

31. The method according to claim 29, wherein the autoimmune disease is selected from the group comprising multiple sclerosis, rheumatoid arthritis and inflammatory bowel disease.

32. The method according to claim 31, wherein the inflammatory bowel disease is selected from the group comprising ulcerative colitis and Crohn's disease.

33. The method according to claim 29, wherein the autoimmune disease is an autoimmune skin disease selected from the group comprising vitiligo, alopecia, atopic dermatitis, Behcet's disease, dermatitis herpetiformis, dermatomyositis, lichen planus, linear IgA disease, lupus of the skin, morphea/scleroderma, ocular cicatrical pemphigoid, pemphigoid, bullous pemphigoid, pemphigoid gestationis, pemphigus, pemphigus vulgaris, pemphigus vulgaris, pemphigus foliaceaous, pemphigus erythematosus, IgA pemphigus, pemphigus vegetans, paraneoplastic pemphigus, vasculitis, epidermolysis bullosa acquisita, psoriasis, and vesiculobullous dermatosis.

34. The method according to claim 33, wherein the autoimmune skin disease is selected from the group comprising vitiligo, segmented or non-segmented vitiligo, alopecia, diffuse alopecia areata, alopecia areata monolocularis, ophiasis alopecia areata, alopecia areata barbae, alopecia areata totallis and alopecia areata universalis.
